(19) United States
(12) Patent Application Publication
(10) Pub. No.: US 2005/0215635 A1
(43) Pub. Date: Sep. 29, 2005

(54) Diarylheptanoid compounds and uses thereof

(76) Inventors: M. Mohamed Rafi, Highland Park, NJ (US); Zhihua Liu, Howell, NJ (US); Robert T. Rosen, Monroe Township, NJ (US); Sharon L. Rosen, (US); Chi-Tang Ho, East Brunswick, NJ (US)

Correspondence Address: JONES DAY, 222 EAST 41ST ST, NEW YORK, NY, 10017, US

(21) Appl. No.: 11/075,275

(22) Filed: Mar. 8, 2005

Related U.S. Application Data
(60) Provisional application No. 60/551,182, filed on Mar. 8, 2004.

(51) Int. Cl. A61K 31/22; A61K 31/12

(52) U.S. Cl. 514/546; 514/690

(57) ABSTRACT

The present invention relates to diarylheptanoid compounds and compositions comprising a diarylheptanoid compound. The present invention also relates to methods for preventing or treating various diseases and disorders by administering to a subject in need thereof one or more diarylheptanoid compounds. In particular, the invention relates to methods for preventing or treating cancer or an inflammatory disorder by administering to a subject in need thereof one or more diarylheptanoid compounds. The present invention further relates to articles of manufacture comprising one or more diarylheptanoid compounds.

This application is entitled to and claims priority to U.S. provisional Ser. No. 60/551,182, filed Mar. 8, 2004, which is incorporated by reference herein in its entirety.

### 1. FIELD OF THE INVENTION

The present invention relates to diarylheptanoid compounds and compositions comprising a diarylheptanoid compound. The present invention also relates to methods for preventing or treating various diseases and disorders by administering to a subject in need thereof one or more diarylheptanoid compounds. In particular, the invention relates to methods for preventing or treating a cell proliferative disorder or an inflammatory disorder by administering to a subject in need thereof one or more diarylheptanoid compounds. The present invention further relates to kits comprising one or more diarylheptanoid compounds.

### 2. BACKGROUND OF THE INVENTION

### 2.1 Cancer and Neoplastic Disease

Cancer is the second leading cause of death in the United States. The American Cancer Society estimated that in 2001, there would be 1.3 million new cases of cancer and that cancer would cause 550,000 deaths. Overall rates have declined by 1% per year during the 1990s. There are 9 million Americans alive who have ever had cancer. NIH estimates the direct medical costs of cancer as $60 billion.

Currently, cancer therapy involves surgery, chemotherapy and/or radiation treatment to eradicate neoplastic cells in a patient (see, for example, Stockdale, 1998, “Principles of Cancer Patient Management”, in Scientific American: Medicine, vol. 3, Rubenstein and Federman, eds., Chapter 12, Section IV). All of these approaches pose significant drawbacks for the patient.

Despite the availability of a variety of chemotherapeutic agents, traditional chemotherapy has many drawbacks (see, for example, Stockdale, 1998, “Principles Of Cancer Patient Management” in Scientific American Medicine, vol. 3, Rubenstein and Federman, eds., ch. 12, sect. 10). Almost all chemotherapeutic agents are toxic, and chemotherapy can cause significant, and often dangerous, side effects, including severe nausea, bone marrow depression, immunosuppression, etc. Additionally, many tumor cells are resistant or develop resistance to chemotherapeutic agents through multi-drug resistance. Therefore, there is a significant need in the art for novel compounds and compositions, and methods that are useful for treating cancer or neoplastic disease with minimal or no side effects. Further, there is a need for cancer treatments that provide cancer-cell-specific therapies with increased specificity and decreased toxicity.

### 2.2 Inflammatory Disorders

Inflammation plays a fundamental role in host defenses and the progression of immune-mediated diseases. The inflammatory response is initiated in response to injury (e.g., trauma, ischemia, and foreign particles) and infection (e.g., bacterial or viral infection) by a complex cascade of events, including chemical mediators (e.g., cytokines and prostaglandins) and inflammatory cells (e.g., leukocytes). The inflammatory response is characterized by increased blood flow, increased capillary permeability, and the influx of phagocytic cells. These events result in swelling, redness, warmth (altered heat patterns), and pus formation at the site of injury or infection.

A delicate well-balanced interplay between the humoral and cellular immune elements in the inflammatory response enables the elimination of harmful agents and the initiation of the repair of damaged tissue. When this delicately balanced interplay is disrupted, the inflammatory response may result in considerable damage to normal tissue and may be more harmful than the original insult that initiated the reaction. In these cases of uncontrolled inflammatory responses, clinical intervention is needed to prevent tissue damage and organ dysfunction. Diseases such as rheumatoid arthritis, osteoarthritis, Crohn's disease, asthma, allergies or inflammatory bowel disease, are characterized by chronic inflammation.

Current treatments for inflammatory disorders involve symptomatic medications and immunosuppressive agents to control symptoms. For example, nonsteroidal anti-inflammatory drugs (NSAIDs) such as aspirin, ibuprofen, fenoprofen, naproxen, tolmetin, sulindac, meclofenamate sodium, piroxicam, flurbiprofen, diclofenac, oxaprozin, nabumetone, etodolac, and ketoprofen have analgesic and anti-inflammatory effects. However, NSAIDs are believed not to be capable of altering progression of the disease. (Tierney et al. (eds), Current Medical Diagnosis & Treatment, 37 ed., Appleton & Lange (1998), p793). Moreover, NSAIDs frequently cause gastrointestinal side effects. Corticosteroids are another class of drugs that are commonly used to control inflammatory symptoms. Corticosteroids, like NSAIDs, do not alter the natural progression of the disease, and thus, clinical manifestations of active disease commonly reappear when the drug is discontinued. Low doses of immunosuppressive agents such as cytotoxic agents are also commonly used to in treatment of inflammatory disorders. Many cytotoxic agents, frequently causes stomatitis, erythema, slopecia, nausea, vomiting, diarrhea, and damage to major organs such kidney and liver. The long-term usage of immunosuppressive agents usually leaves the patient defenseless to infections.

New treatment and preventative modalities for inflammatory disorders are constantly being sought. In particular, any new modalities that reduces the dosage and/or frequency of administration of agents currently being used, or is capable of making a currently used treatment and/or prevention more effective is constantly being sought.

The use of herbal therapy or alternative medicine is becoming an increasingly attractive approach for the treatment of various inflammatory disorders. The majority of naturally occurring phenolics in different plants possess tremendous antioxidative and anti-inflammatory activities (Surh et al., 2001). Anti-inflammatory properties of various phytochemicals are mediated through the inhibition of production of cytokines (IL-1β, TNF-α, IL-6, IL-12, IFN-γ), nitric oxide (NO), prostaglandins and leukotriens. Antioxidants such as (−)-epigallocatechin-3-gallate (EGCG) (Lin and Lin, 1997), resveratrol (Tsai et al., 1999) and naturally occurring flavonoids including apigenin and kaempferol (Liang et al., 1999) have been reported to suppress NO production through inhibition of nuclear factor-kappa B (NF-κB). Earlier studies have shown that ginger and its constituents are potent inhibitors of immune cell activation and cytokine secretion and used for the treatment of cancer (Ageel et al., 1989). Pharmacologically, ginger (Zingiber officinale), similar to other plants, possess very complex mixture of compounds. This plant contains several hundred known constituents; among them are gingerols, beta-carotene, capsaicin, caffeic acid, and curcumin. Various formulations of ginger have been shown to act as a dual inhibitor of both cyclooxygenase (COX) and lipooxygenase (Mustafa et al., 1993), to inhibit leukotriene synthesis (Kikuchi et al., 1992), and to reduce carrageenan-induced rat-paw edema (Mascolo et al., 1989; Jana et al., 1999). Another closely related plant, commonly known as Greater galanga (Alpinia galanga), had also traditionally been used for rheumatic conditions in South East Asian medicine. The German Commission E Monographs lists the use of Lesser galangal (Alpinia officinarum), which is closely related to Alpinia galanga, for dyspepsia and loss of appetite. The US Food and Drug Administration list Zingiber officinale and Alpinia officinarum as “generally regarded as safe” (21 CFR Section 182.10, 182.20). Various preparations from Lesser galangal have been used as traditional medicine in China due to its significant therapeutic properties for spleen and stomach. Most important compounds identified in lesser galangal are flavonoids and diarylheptanoids. The various gingerols and diarylheptanoids, naturally occurring in Lesser galangal, have been shown to be a potent inhibitor of prostaglandin synthase enzymatic activity (Kiuchi et al., 1992).

The critical role of NO in various pathological conditions has led to the discovery of new therapeutic agents from varied sources Nitric oxide (NO) is a short-lived free radical produced from L-arginine in a reaction catalyzed by NO synthase (NOS). It mediates diverse functions by acting on most cells of the body through the interaction with different molecular targets, which can either be activated or inhibited (Xie and Fidler, 1998). At least three types of NOS isoforms have been reported (Nathan and Xie, 1994a). Endothelial NOS and neuronal NOS are constitutively expressed and are Ca²⁺/calmodulin dependent. Whereas, the high-output isoform, inducible NOS (iNOS), is expressed by cytokines such as interferon (IFN) α, β and -γand interleukin (IL)-1α and -1β, and lipopolysaccharide (LPS)-activated macrophages and endothelial cells following their transcriptional induction and new protein synthesis (Nathan and Xie, 1994b). Low concentrations of NO produced by iNOS possess beneficial roles in antimicrobial activity of macrophages against pathogens (Cook and Cattell, 1996). At the same time excessive production of NO and its derivatives, such as peroxynitrite and nitrogen dioxide, have been suggested to be mutagenic in vivo, provoke the pathogenesis of septic shock and diverse autoimmune disorders (Nguyen et al., 1992; Wink et al., 1991; Miller et al., 1993; Kilbourn et al., 1990). Furthermore, NO and its oxidized forms have also been shown to be carcinogenic (Halliwell, 1994). Therefore, suppressing high NO production by inhibiting iNOS expression or its activity may be a therapeutic tool for management of NO-related disorders.

Citation of any reference in Section 2 of this application is not to be construed as an admission that such reference is prior art to the present application.

### 3. SUMMARY OF THE INVENTION

The present invention provides methods of using diarylheptanoid compounds in the prevention, treatment or management of various disorders. The present invention provides certain novel diarylheptanoid compounds as well as novel compositions comprising such compounds, such as but not limited to dietary supplements, cosmetic compositions, food additives, and pharmaceutical compositions.

In certain aspects, the present invention provides compounds having the formulas Ia and Ib, as described below: 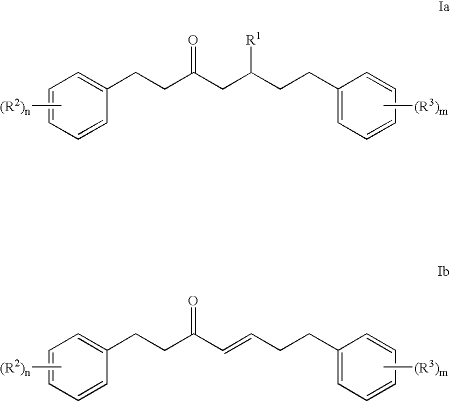
or a pharmaceutically acceptable salt, solvate or hydrate thereof,
wherein
R¹ is hydroxy, alkoxy or acyloxy;
   each R²is independently hydroxy, alkoxy or acyloxy;
   each R³ is independently hydroxy, alkoxy or acyloxy;
   each n is independently an integer from 0 to 5;
   and each m is independently an integer from 0 to 5.

In particular embodiments, the present invention provides compounds according to formulas Ia and IIb as described below: 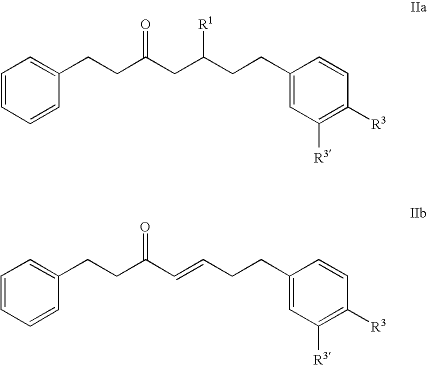
and pharmaceutically acceptable salts, solvates and hydrates thereof,
wherein
R¹ is hydroxy, alkoxy or acyloxy;
   each R² is independently hydroxy, alkoxy or acyloxy;
   each R³ is independently hydroxy, alkoxy or acyloxy.

In particular embodiments, the present invention provides 5-hydroxy-7-(4′-hydroxy-3′methoxyphenyl)-1-phenyl-3-heptanone, 5-methoxy-7-(4′-hydroxy-3′methoxyphenyl)-1-phenyl-3-heptanone, 7-(4′-hydroxyphenyl)-1-phenyl-hept-4-en-3-one, 7-(4′-hydroxy-3′-methoxyphenyl)-1-phenyl-hept-4-en-3-one, and 1,7-diphenylhept-4-en-3-one, respectively having the following formula:

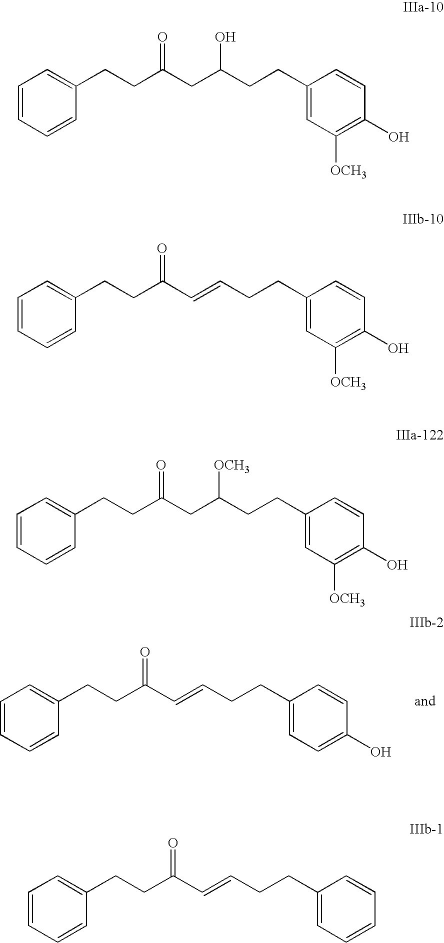

The present invention also provides compositions comprising one or more compounds of the invention. In general, the composition is not a natural source of such compounds, such as anatomical parts of the Alpina officinarum plant. In one aspect, a composition of the invention comprises a mixture of diarylheptanoids, including one or more of the compounds of formula Ia and/or formula Ib, or pharmaceutically acceptable salts, solvates or hydrates thereof, wherein (i) the concentration of a diarylheptanoid in the composition is different from that in a natural source of the diarylhaptanopid; and/or (ii) that the ratio of the concentration of one diarylheptanoid in the composition to that of another diarylheptanoid in the composition is different from that in a natural source of the diarylheptanoids.

Such a composition can be prepared, for example, by processing a natural source of diarylheptanoids such that at least one particular diarylheptanoid has been selectively removed, retained, or enriched. Alternatively, one or more purified diarylheptanoids can be used to make such compositions. Such a composition can also be prepared, for example, by adding an amount of at least one diarylheptanoid to a natural source or processed form of a natural source of the diarylheptanoids.

In various embodiments, a composition of the invention can comprise a mixture of compounds having the formula Ia and/or formula Ib, wherein (i) the concentration of one or more of the compounds having the formula Ia is increased or decreased relative to that in a natural source of the compounds; (ii) the concentration of one or more of the compounds having the formula Ib is increased or decreased relative to that found in a natural source of the compounds.

In specific embodiments, a composition of the invention can comprise a mixture of compounds having the formula Ia and/or formula Ib, wherein (i) the concentration of one or more of the compounds having the formula Ia is increased or decreased relative to that in a natural source of the compounds; (ii) the concentration of one or more of the compounds having the formula IIb is increased or decreased relative to that found in a natural source of the compounds.

In yet another embodiment, the invention provides a composition comprising a mixture of compounds having the formula Ia, formula Ib, formula Ia, and/or formula IIb, or pharmaceutically acceptable salts, solvates or hydrates thereof, including but not limited to the compounds of formula IIIa and IIIb described in Section 5, or pharmaceutically acceptable salts, solvates or hydrates thereof, wherein the percentages (by dry weight) of one or more diarylheptanoids relative to the total content of diarylheptanoids in the composition is different from that in a natural source of the diarylheptanoids.

In another embodiment, a composition of the invention can comprise a mixture of compounds having the formula Ia, formula Ib, formula IIa, and/or formula IIb, or pharmaceutically acceptable salts, solvates or hydrates thereof, including but not limited to the compounds of formula IIIa and IIIb, or pharmaceutically acceptable salts, solvates or hydrates thereof, wherein the ratio of certain diarylheptanoids in the composition is different from that in a natural source.

In another aspect, the present invention also provides pharmaceutical compositions comprising one or more compounds of the invention, or a pharmaceutically acceptable salt, solvate, or hydrate thereof. The present invention provides pharmaceutical compositions comprising one or more compounds of the invention, or a pharmaceutically acceptable salt, solvate, or hydrate thereof, and one or more prophylactic or therapeutic agents in addition to the diarylheptanoids of the compositions. In one embodiment, such prophylactic or therapeutic agents contribute to the prevention, treatment or amelioration of a proliferative disorder or an inflammatory disorder, or one or more symptoms thereof.

In yet another aspect, the present invention also provides food additives, dietary supplements, nutraceutical compositions and food compositions comprising one or more compounds or compositions of the invention. In one embodiment, the food additives, dietary supplements, nutraceutical compositions and/or food compositions of the invention are prepared from natural sources.

The compositions, food additives, dietary supplements, and food compositions of the invention can comprise a mixture of diarylheptanoid compounds having the formula Ia, formula Ib, formula IIa, and/or formula IIb, or pharmaceutically acceptable salts, solvates or hydrates thereof, including but not limited to the compounds of formula IIIa and IIIb described in Section 5, or pharmaceutically acceptable salts, solvates or hydrates thereof, wherein the ratio of certain diarylheptanoids in the composition is different from that of a natural source. The compositions, food additives, dietary supplements, and food compositions of the invention can comprise a mixture of compounds having the formula Ia, formula Ib, formula Ia, and/or formula IIb, or pharmaceutically acceptable salts, solvates or hydrates thereof, including but not limited to the compounds of formula IIIa and IIIb, or pharmaceutically acceptable salts, solvates or hydrates thereof, wherein the percentages (by dry weight) of one or more diarylheptanoids relative to the total content of diarylheptanoids is different from that in a natural source of the diarylheptanoids.

In yet another aspect, the invention provides methods for the prevention, treatment, management, or amelioration of proliferative disorders or inflammatory disorders, or one or more symptoms thereof, said methods comprising administering to a subject in need thereof a prophylactically or therapeutically effective amount of one or more compounds of the invention. Administration of such compounds can, for example, be via one or more of the compositions, food additives, dietary supplements, nutraceutical compositions or food compositions of the invention.

The invention also provides methods for the prevention, treatment, management, or amelioration of proliferative disorders or inflammatory disorders, or one or more symptoms thereof, said methods comprising administering to a subject in need thereof a prophylactically or therapeutically effective amount of one or more compounds of the invention and a prophylactically or therapeutically effective amount of at least one other therapy (e.g., at least one other prophylactic or therapeutic agent) other than a compound of the invention. Non-limiting examples of such agents include anti-viral agents, antibiotic agents, anti-angiogenic agents, TNF-α antagonists, immunomodulatory agents, anti-cancer agents, and anti-inflammatory agents. Administration of such a combination of compounds can, for example, be via one or more of the compositions, food additives, dietary supplements, or food compositions of the invention.

The invention also provides methods for preventing or treating an adverse health condition associated with the activation or nuclear translocation of nuclear factor-kappa B (NF-κB) or the binding of NF-κB to DNA in cells of a subject having the adverse health condition. Examples of adverse health condition can be an inflammatory disorder, a proliferative disorder, or a cancer, or any symptoms associated with such disorders and cancer. The methods comprise administering compositions or compounds of the invention to the subject or contacting the subject with compositions or compounds of the invention. In specific embodiments, the composition is a pharmaceutical composition which further comprises at least a pharmaceutical carrier, an immunomodulatory agent, an anti-angiogenic agent, a TNF-α antagonist, an anti-inflammatory agent, an anti-cancer agent, an antibiotic, an anti-histamine, or an anti-viral agent.

The invention also provides methods for relieving a symptom of inflammation in a subject, such as but not limited to redness, swelling, edema, excess warmth, and can be associated with asthma, allergic reaction, allergic disorder, fibrotic disease, psoriasis, multiple sclerosis, systemic lupus erythrematosis, chronic obstructive pulmonary disease, inflammatory bowel disease, ischemic reperfusion injury, gout, Behcet's disease, septic shock, undifferentiated spondyloarthropathy, undifferentiated arthropathy, arthritis, juvenile rheumatoid arthritis, adult rheumatoid arthritis, osteoarthritis, psoriatic arthritis, inflammatory osteolysis, chronic viral infection or chronic bacterial infection. Such methods comprise contacting the subject with a composition or a compound of the invention or administering a composition or a compound of the invention to the subject. In specific embodiements, the composition is a dietary supplement and further comprises at least a consumable carrier, a vitamin, an amino acid, an antioxidant, a botanical extract, or a flavoring agent.

In another aspect, the present invention provides articles of manufacture comprising, in one or more containers, a compound, composition, dietary supplement, food additive or food composition of the invention.

### 3.1 Terminology and Abbreviations

As used herein, “a” or “an” means at least one, unless clearly indicated otherwise. The term “about,” unless otherwise indicated, refers to a value that is no more than 10% above or below the value being modified by the term.

As used herein, the terms “antibody” and “antibodies” refer to molecules that contain an antigen binding site, e.g., immunoglobulins. Immunoglobulin molecules can be of any type (e.g., IgG, IgE, IgM, IgD, IgA and IgY), class (e.g., IgG₁, IgG₂, IgG₃, IgG₄, IgA₁ and IgA₂) or subclass. Antibodies include, but are not limited to, monoclonal antibodies, multispecific antibodies, human antibodies, humanized antibodies, camelised antibodies, chimeric antibodies, single domain antibodies, single chain Fvs (scFv), single chain antibodies, Fab fragments, F(ab′) fragments, disulfide-linked Fvs (sdFv), and anti-idiotopic (anti-Id) antibodies (including, e.g., anti-Id antibodies to antibodies of the invention), and epitope-binding fragments of any of the above.

As used herein, unless indicated otherwise, the terms “compound” and “compound of the invention,” are used interchangeably to refer to the compounds of Formula Ia, Ib, Ia, IIb, IIIa and IIIb (including compounds IIIa-1 through to III-630, and compounds IIIb-1 through to IIIb-630).

As used herein, the terms “disorder” and “disease” are used interchangeably to refer to a condition in a subject. Certain conditions may be characterized as more than one disorder. For example, certain conditions may be characterized as both non-cancerous proliferative disorders and inflammatory disorders.

As used herein, the term “effective amount” refers to the amount of a compound of the invention which is sufficient to reduce or ameliorate the severity, duration of a disorder (e.g., a proliferative disorder or a disorder characterized by inflammation (i.e., an inflammatory disorder) or one or more symptoms thereof, prevent the advancement of a disorder (e.g., a proliferative disorder or an inflammatory disorder), cause regression of a disorder (e.g., a proliferative disorder or an inflammatory disorder), prevent the recurrence, development, or onset of one or more symptoms associated with a disorder (e.g., a proliferative disorder or an inflammatory disorder), or enhance or improve the prophylactic or therapeutic effect(s) of another therapy.

As used herein, the term “in combination” refers to the use of more than one therapies (e.g., one or more prophylactic and/or therapeutic agents). The use of the term “in combination” does not restrict the order in which therapies (e.g., prophylactic and/or therapeutic agents) are administered to a subject with a disorder (e.g., a proliferative disorder or an inflammatory disorder). A first therapy (e.g., a prophylactic or therapeutic agent such as a compound of the invention) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent such as an anti-inflammatory agent or anti-angiogenic agent) to a subject with a disorder (e.g., a proliferative disorder or an inflammatory disorder).

As used herein, the term “isolated” in the context of a compound such as, e.g., a compound of the invention, refers to a compound that is substantially free of chemical precursors or other chemicals when chemically synthesized. In a specific embodiment, the compound is 60%, 65%, 75%, 80%, 85%, 90%, 95%, or 99% free (by dry weight) of other, different compounds. Preferably, the compounds of the invention are isolated.

As used herein, the term “isolated” in the context of a compound that can be obtained from a natural source, e.g., plants, refers to a compound which is substantially free of natural source cellular material, e.g., plant cellular material, or contaminating materials from the natural source, e.g., cell or tissue source, from which it is obtained. The language “substantially free of natural source cellular material” or substantially free of plant cellular material” includes preparations of a compound that has been separated from cellular components of the cells from which it is isolated. Thus, a compound that is substantially free of cellular material (e.g., natural source cellular material, such as plant cellular material) includes preparations of a compound having less than about 30%, 20%, 10%, or 5% (by dry weight) of heterologous materials, e.g., phytochemicals, (also referred to as a “contaminating materials”).

As used herein, the terms “manage,” “managing,” and “management” refer to the beneficial effects that a subject derives from a therapy (e.g., a prophylactic or therapeutic agent), while not resulting in a cure of the disease. In certain embodiments,.a subject is administered one or more therapies (e.g., one or more prophylactic or therapeutic agents) to “manage” a disease so as to prevent the progression or worsening of the disease.

As used herein, the terms “non-responsive” and “refractory” describe patients treated with a currently available therapy (e.g., a prophylactic or therapeutic agent) for a disorder (e.g., a proliferative disorder or an inflammatory disorder), which is not clinically adequate to relieve one or more symptoms associated with such disorder. Typically, such patients suffer from severe, persistently active disease and require additional therapy to ameliorate the symptoms associated with their disorder (e.g., a proliferative disorder or an inflammatory disorder).

As used herein, the phrase “pharmaceutically acceptable salt” refers to pharmaceutically acceptable organic or inorganic salts of a compound of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p toluenesulfonate, and pamoate (i.e., 1,1′ methylene bis (2 hydroxy 3 naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counterions. Hence, a pharmaceutically acceptable salt can have one or more charged atoms and/or one or more counterion.

As used herein, the term “pharmaceutically acceptable solvate” refers to an association of one or more solvent molecules and a compound of the invention. Examples of solvents that form pharmaceutically acceptable solvates include, but are not limited to, water, isopropanol, ethanol, methanol, DMSO, ethyl acetate, acetic acid, and ethanolamine.

As used herein, the terms “prophylactic agent” and “prophylactic agents” as used refer to any agent(s) which can be used in the prevention of a disorder (e.g., a proliferative disorder or an inflammatory disorder) or one or more symptoms thereof. In certain embodiments, the term “prophylactic agent” refers to a compound of the invention. In certain other embodiments, the term “prophylactic agent” does not refer to a compound of the invention. Prophylactic agents may be characterized as different agents based upon one or more effects that the agents have in vitro and/or in vivo.

As used herein, the terms “prevent,” “preventing” and “prevention” refer to the prevention of the recurrence, onset, or development of a disorder or a symptom thereof in a subject resulting from the administration of a therapy (e.g., a prophylactic or therapeutic agent), or the administration of a combination of therapies (e.g., a combination of prophylactic or therapeutic agents).

As used herein, the phrase “prophylactically effective amount” refers to the amount of a therapy (e.g., prophylactic agent) which is sufficient to result in the prevention of the development, recurrence or onset of a disorder or a symptom thereof associated with a disorder (e.g., a proliferative disorder or an inflammatory disorder), or to enhance or improve the prophylactic effect(s) of another therapy (e.g., another prophylactic agent). Examples of prophylactically effective amounts of compounds are provided infra.

As used herein, the phrase “side effects” encompasses unwanted and adverse effects of a therapy (e.g., a prophylactic or therapeutic agent). Side effects are always unwanted, but unwanted effects are not necessarily adverse. An adverse effect from a therapy (e.g., prophylactic or therapeutic agent) might be harmful or uncomfortable or risky. Side effects include, but are not limited to fever, chills, lethargy, gastrointestinal toxicities (including gastric and intestinal ulcerations and erosions), nausea, vomiting, neurotoxicities, nephrotoxicities, renal toxicities (including such conditions as papillary necrosis and chronic interstitial nephritis), hepatic toxicities (including elevated serum liver enzyme levels), myelotoxicities (including leukopenia, myelosuppression, thrombocytopenia and anemia), dry mouth, metallic taste, prolongation of gestation, weakness, somnolence, pain (including muscle pain, bone pain and headache), hair loss, asthenia, dizziness, extra pyramidal symptoms, akathisia, cardiovascular disturbances and sexual dysfunction.

As used herein, the terms “subject” and “patient” are used interchangeably herein. The terms “subject” and “subjects” refer to an animal, preferably a mammal including a non-primate (e.g., a cow, pig, horse, cat, dog, rat, and mouse) and a primate (e.g., a monkey such as a cynomolgous monkey, a chimpanzee and a human), and more preferably a human. In one embodiment, the subject is refractory or non-responsive to current treatments for a disorder (e.g., a proliferative disorder or an inflammatory disorder). In another embodiment, the subject is an animal that a veterinarian sees. In another embodiment, the subject is a farm animal (e.g., a horse, a cow, a pig, etc.) or a pet (e.g., a dog or a cat). In another embodiment, the subject is an immunocompromised or immunosuppressed mammal, preferably a human. In an alternative embodiment, the subject is not an immunocompromised or immunosuppressed mammal, preferably a human (e.g., an HIV patient). In another embodiment, the subject is an animal, preferably a mammal, and more preferably a human, that is predisposed and/or at risk because of a genetic factor(s), an environmental factor(s), or a combination thereof to develop a disorder. In certain embodiments, the subject is not an animal, e.g., a mouse or a rat, including a mouse or rat that has been modified, either genetically or otherwise, to be useful as an animal model for a disorder.

As used herein, the term “synergistic” refers to a combination of compounds of the invention and/or a combination of a compound or compounds of the invention and another therapy (e.g., a prophylactic or therapeutic agent), including one which has been or is currently being used to prevent, manage or treat a disorder (e.g., a proliferative disorder or an inflammatory disorder), which combination is more effective than the additive effects of the individual compounds or therapies. A synergistic effect of a combination of therapies (e.g., a combination of prophylactic or therapeutic agents) can permit the use of lower dosages of one or more of the therapies and/or less frequent administration of said therapies to a subject with a disorder (e.g., a proliferative disorder or an inflammatory disorder). The ability to utilize lower dosages of a therapy (e.g., a prophylactic or therapeutic agent) and/or to administer said therapy less frequently can reduce the toxicity associated with the administration of said therapy to a subject without reducing the efficacy of said therapy in the prevention, management or treatment of a disorder (e.g., a proliferative disorder or an inflammatory disorder). In addition, a synergistic effect can result in improved efficacy of agents in the prevention, management or treatment of a disorder (e.g., a proliferative disorder or an inflammatory disorder). Moreover, a synergistic effect of a combination of therapies (e.g., a combination of prophylactic or therapeutic agents) can avoid or reduce adverse or unwanted side effects associated with the use of either therapy alone.

As used herein, the terms “therapeutic agent” and “therapeutic agents” refer to any agent(s) which can be used in the treatment, management, or amelioration of a disorder (e.g., a proliferative disorder or an inflammatory disorder) or one or more symptoms thereof. In certain embodiments, the term “therapeutic agent” refers to a compound of the invention. In certain other embodiments, the term “therapeutic agent” refers does not refer to a compound of the invention. Therapeutic agents may be characterized as different agents based upon one or more effects the agents have in vivo and/or in vitro, for example, an anti-inflammatory agent may also be characterized as an immunomodulatory agent.

As used herein, the term “therapeutically effective amount” refers to that amount of a therapy (e.g., a therapeutic agent) sufficient to result in the amelioration of one or more symptoms of a disorder (e.g., a proliferative disorder or an inflammatory disorder), prevent advancement of a disorder (e.g., a proliferative disorder or an inflammatory disorder), cause regression of a disorder (e.g., a proliferative disorder or an inflammatory disorder), or to enhance or improve the therapeutic effect(s) of another therapy. In a specific embodiment, with respect to the treatment of cancer, an effective amount refers to the amount of a therapy (e.g., a therapeutic agent) that inhibits or reduces the proliferation of cancerous cells, inhibits or reduces the spread of tumor cells (metastasis), inhibits or reduces the onset, development or progression of cancer or a symptom thereof, or reduces the size of a tumor. Preferably, a therapeutically effective of a therapy (e.g., a therapeutic agent) reduces the proliferation of cancerous cells or the size of a tumor by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%, relative to a control or placebo such as phosphate buffered saline (“PBS”). In another embodiment, with respect to inflammation, an effective amount refers to the amount of a therapy (e.g., a therapeutic agent) that reduces the inflammation of a joint, organ or tissue. Preferably, a therapeutically effective of a therapy (e.g., a therapeutic agent) reduces the inflammation of a joint, organ or tissue by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%, relative to a control or placebo such as phosphate buffered saline. In another embodiment, with respect to the treatment of psoriasis, an effective amount preferably refers to the amount of a therapy (e.g., therapeutic agent) that reduces a human's Psoriasis Area and Severity Index (PASI) score by at least 20%, at least 35%, at least 30%, at least 40%, at least 45%, at least 50%, at least 55%, at least 60%, at least 65%, at least 70%, at least 75%, at least 80%, or at least 85%. In an alternative embodiment, with respect to the treatment of psoriasis, an effective amount preferably refers to the amount of a therapy (e.g., a therapeutic agent) that improves a human's global assessment score by at least 25%, at least 35%, at least 30%, at least 40%, at least 45%, at least 50%, at least 55%, at least 60%, at least 65%, at least 70%, at least 75%, at least 80%, at least 85%, at least 90%, or at least 95%. Examples of therapeutically effective amounts of compounds are provided infra.

As used herein, the terms “therapies” and “therapy” can refer to any protocol(s), method(s), and/or agent(s) that can be used in the prevention, treatment, management, or amelioration of a disorder (e.g., a proliferative disorder or an inflammatory disorder) or one or more symptoms thereof. In certain embodiments, the terms “therapy” and “therapies” refer to chemotherapy, radiation therapy, hormonal therapy, biological therapy, and/or other therapies useful in the prevention, management, treatment or amelioration of a disorder (e.g., a proliferative disorder or an inflammatory disorder) or one or more symptoms thereof known to one of skill in the art (e.g., skilled medical personnel).

As used herein, the terms “treat”, “treatment” and “treating” refer to the reduction or amelioration of the progression, severity and/or duration of a disorder (e.g., a proliferative disorder or an inflammatory disorder), or the amelioration of one or more symptoms thereof resulting from the administration of one or more therapies (e.g., one or more therapeutic agents such as a compound of the invention). In specific embodiments, such terms refer to the inhibition or reduction in the proliferation of cancerous cells, the inhibition or reduction in the spread of tumor cells (metastasis), the inhibition or reduction in the onset, development or progression of cancer or a symptom thereof, the reduction in the size of a tumor, or the improvement in a patient's ECOG or Karnofsky score. In other embodiments, such terms refer to a reduction in the swelling of one or more joints, organs or tissues, or a reduction in the pain associated with an inflammatory disorder. In yet other embodiments, such terms refer to a reduction a human's PASI score or an improvement in a human's global assessment score.

When describing the compounds, compositions (including pharmaceutical, cosmetic compositions, dietary supplements, or food additives) containing such compounds and methods of using such compounds and compositions, the following terms have the following meanings unless otherwise indicated. It should also be understood that, consistent with the scope of the present invention, any of the moieties defined herein and/or set forth below may be substituted with a variety of substituents, and that the respective definitions are intended to include such substituted moieties within their scope. In preferred embodiments, the moieties are unsubstituted.

“Acyl” refers to a radical —C(O)R, where R is hydrogen, alkyl or cycloalkyl, as defined herein. Representative examples include, but are not limited to, formyl, acetyl, cylcohexylcarbonyl, cyclohexylmethylcarbonyl, and the like.

“Acyloxy” refers to the group —OC(O)R where R is hydrogen, alkyl or cycloalkyl.

“Aliphatic” refers to hydrocarbyl organic compounds or groups characterized by a straight, branched or cyclic arrangement of the constituent carbon atoms and an absence of aromatic unsaturation. Aliphatics include, without limitation, alkyl, alkylene, alkenyl, alkenylene, alkynyl and alkynylene. Aliphatic groups typically have from 1 or 2 to about 12 carbon atoms.

“Alkyl” refers to monovalent saturated aliphatic hydrocarbyl groups particularly having up to about 11 carbon atoms, more particularly as a lower alkyl, from 1 to 8 carbon atoms and still more particularly, from 1 to 6 carbon atoms. The hydrocarbon chain may be either straight-chained or branched. This term is exemplified by groups such as methyl, ethyl, n-propyl, isopropyl, n-butyl, iso-butyl, tert-butyl, n-hexyl, n-octyl, tert-octyl and the like. The term “lower alkyl” refers to alkyl groups having 1 to 6 carbon atoms. The term “alkyl” also includes “cycloalkyl” as defined below.

“Substituted alkyl” refers to an alkyl group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, carboxyl, cycloalkyl, substituted cycloalkyl, halogen, hydroxyl and keto.

“Alkoxy” refers to the group —OR where R is alkyl. Particular alkoxy groups include, by way of example, methoxy, ethoxy, n-propoxy, isopropoxy, n-butoxy, tert-butoxy, sec-butoxy, n-pentoxy, n-hexoxy, 1,2-dimethylbutoxy, and the like.

“Substituted alkoxy” includes those groups recited in the definition of “substituted” herein, and particularly refers to an alkoxy group having 1 or more substituents, for instance from 1 to 5 substituents, and particularly from 1 to 3 substituents, selected from the group consisting of acyl, acylamino, acyloxy, alkoxy, substituted alkoxy, alkoxycarbonyl, carboxyl, cycloalkyl, substituted cycloalkyl, halogen, hydroxyl and keto.

“Cycloalkyl” refers to cyclic hydrocarbyl groups having from 3 to about 10 carbon atoms and having a single cyclic ring or multiple condensed rings, including fused and bridged ring systems, which optionally can be substituted with from 1 to 3 alkyl groups. Such cycloalkyl groups include, by way of example, single ring structures such as cyclopropyl, cyclobutyl, cyclopentyl, cyclooctyl, 1-methylcyclopropyl, 2-methylcyclopentyl, 2-methylcyclooctyl, and the like, and multiple ring structures such as adamantanyl, and the like.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

### 4. BRIEF DESCRIPTION OF THE FIGURES

FIG. 1. Reversed phase HPLC chromatogram of Alpinia officinarum 30:1 chloroform/methanol fraction at 205 nm using a Discovery C18 reversed phase HPLC column (250 mm×4.6 mm, 5 μm).

FIG. 2. Effect of 7-(4″-hydroxy-3″-methoxyphenyl)-1-phenyl-hept-4-en-3-one (referred to herein also as AO-4 or compound IIIb-10 or HMP) on RAW 264.7 cell viability.

FIG. 3. Inhibition of LPS induced iNOS and COX-2 protein expression by HMP. RAW 264.7 cells were treated with various concentrations of HMP and/or LPS (0.5 μg/ml) for 18 h. Total-cell lysate (40 μg) was resolved in 8-10% SDS-PAGE then transferred to PVDF membrane and detected with specific antibodies as described in Section 6. This experiment was repeated 4 times with similar observations. Lane-1, Control (without any treatment); 2, LPS (0.5 μg/ml); 3, LPS and HMP 25 μM; 4, LPS and HMP-12.5 μM.

FIG. 4A and B: Effect of HMP on mRNA expression of iNOS and COX-2. Fig. A, RAW 264.7 cells were incubated in the presence of LPS (0.5 μg/ml) with or without HMP (12.5, 25 μM) for 12 hrs. Total RNA isolated from RAW 264.7 cells after 12 hrs of treatments with LPS alone or with various concentration of HMP was reverse transcribed to make cDNA and 2 μl (for iNOS) or 1 μl (for COX-2) of cDNA was amplified using gene specific primer (Ambion Inc). Ten μl of each PCR reaction was resolved in 2% agarose gel. Lane 1, control; 2. LPS (0.5 μg/ml); 3, LPS+HMP (12.5 μM) and Lane, 4 LPS+HMP (25 μM). Fig. B, Histogram showing densitometric analysis of iNOS and COX-2 mRNA expression. Each histogram shows the ratio of integrated density of iNOS or COX-2 mRNA with 18 S ribosomal RNA in the sample. This experiment was repeated 3 times separately with similar observation.

FIG. 5A and 5B. Effect of HMP on LPS induced MAPKs activation. FIG. 5A, Total protein lysates of RAW 264.7 were prepared after treatment with HMP and/or LPS for indicated times and 40 μg of protein lysates were resolved and probed for phosphorylated (pp44/42 and pp38) and basal p44/42 and p38, using specific monoclonal antibodies as described in material and methods. FIG. 5B, Integrated density of phosphorylated p44/42 (pp44/42) as measured by Scion software. This experiment was repeated 4 times with similar results.

FIG. 6A and 6B. Inhibition of LPS induced NF-κB activation by HMP. 6A: Nuclear protein lysates of RAW 264.7 cells were prepared after 2 hrs of treatment with LPS (0.5 μg/ml) either alone or with different concentration of HMP. Six μg of nuclear protein was used in DNA binding assay and the total binding reaction was resolved in 5% nondenaturing polyacrylamide gel. The retarded bands are indicated with arrow. Data represent one of three experiments performed separately with similar results. Lane 1, control; 2, LPS (0.5 μg/ml); 3, LPS+HMP (25 μM); 4, HMP (12.5 μM). NS, non specific. 6B: Plot of integrated signal density for each lane in FIG. 6A.

### 5. DETAILED DESCRIPTION OF THE INVENTION

### 5.1 The Compounds of the Invention

The present invention provides compositions comprising one or more compounds having formulas Ia, Ib, Ia and IIb, and methods of their use and methods of their preparation. The compositions and methods are described in detail in the sections below.

In one aspect, the compounds have the formulas Ia and Ib, as described below: 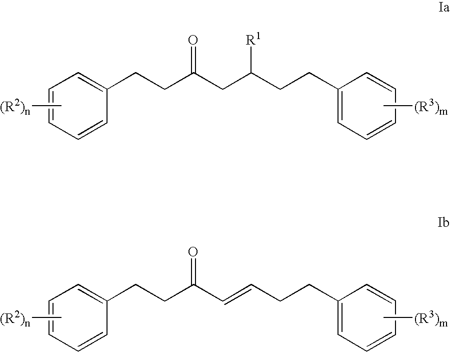
or a pharmaceutically acceptable salt, solvate or hydrate thereof,
wherein
   R¹ is hydroxy, alkoxy or acyloxy;
   each R² is independently hydroxy, alkoxy or acyloxy;
   each R³ is independently hydroxy, alkoxy or acyloxy;
   each n is independently an integer from 0 to 5;
   and each m is independently an integer from 0 to 5.

In particular embodiments, the present invention provides compounds according to formulas Ia and IIb as described below: 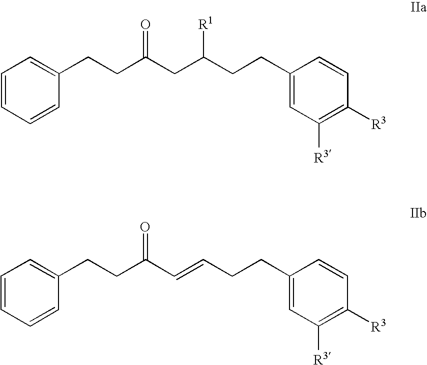
and pharmaceutically acceptable salts, solvates and hydrates thereof,
wherein
   R¹ is hydroxy, alkoxy or acyloxy;
   each R³is independently hydroxy, alkoxy or acyloxy;
   each R³ is independently hydroxy, alkoxy or acyloxy.

When R¹ is acyloxy, R¹ can be any acyloxy known to those of skill in the art. For instance, R¹ can be derived from any fatty acid, naturally occurring or otherwise, known to those of skill in the art. In preferred embodiments when R¹ is acyloxy, R¹ is acetoxy.

When R² is acyloxy, R² can be any acyloxy known to those of skill in the art. For instance, R² can be derived from any fatty acid, naturally occurring or otherwise, known to those of skill in the art. In preferred embodiments when R² is acyloxy, R² is acetoxy.

When R³ is acyloxy, R³ can be any acyloxy known to those of skill in the art. For instance, R³ can be derived from any fatty acid, naturally occurring or otherwise, known to those of skill in the art. In preferred embodiments when R³ is acyloxy, R³ is acetoxy.

When R³′ is acyloxy, R³′ can be any acyloxy known to those of skill in the art. For instance, R³′ can be derived from any fatty acid, naturally occurring or otherwise, known to those of skill in the art. In preferred embodiments when R³′ is acyloxy, R³′ is acetoxy.

When R¹ is alkoxy, R¹ can be any alkoxy known to those of skill in the art. For instance, R¹ can be, in certain embodiments, an alkoxy group comprising a lower alkyl group. In preferred embodiments when R¹ is alkoxy, R¹ is methoxy.

When R² is alkoxy, R² can be any alkoxy known to those of skill in the art. For instance, R¹ can be, in certain embodiments, an alkoxy group comprising a lower alkyl group. In preferred embodiments when R² is alkoxy, R² is methoxy.

When R³ is alkoxy, R³ can be any alkoxy known to those of skill in the art. For instance, R³ can be, in certain embodiments, an alkoxy group comprising a lower alkyl group. In preferred embodiments when R³ is alkoxy, R³ is methoxy.

When R³′ is alkoxy, R³′ can be any alkoxy known to those of skill in the art. For instance, R³′ can be, in certain embodiments, an alkoxy group comprising a lower alkyl group. In preferred embodiments when R³′ is alkoxy, R³′ is methoxy.

In certain embodiments, n is 0 and m is an integer from 1 to 5. In particular embodiments, n is 0 and m is an integer from 2 to 5. In preferred embodiments, n is 0 and m is 2.

In certain embodiments where n is 0 and m is 2, each R³ or R³′ is hydroxy or alkoxy. In preferred embodiments, the alkoxy is C₁-C₇ alkoxy. For instance, the alkoxy can be C₁, C₂ or C₃ alkoxy. In particular embodiments, the alkoxy is methoxy.

Illustrative examples of these compounds are set forth below in formula IIIa or IIIb:

| | |
|---|---|
| | IIIa |
| 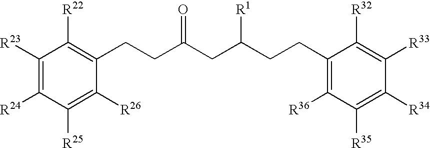 | |
| | IIIb |
| 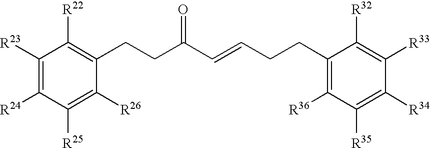 | |

| # | R¹ | R²² | R²³ | R²⁴ | R³² | R³³ | R³⁴ |
|---|---|---|---|---|---|---|---|
| 1 | OH | H | H | H | H | H | H |
| 2 | OH | H | H | H | H | H | OH |
| 3 | OH | H | H | H | H | H | OMe |
| 4 | OH | H | H | H | H | H | OAc |
| 5 | OH | H | H | H | H | OH | H |
| 6 | OH | H | H | H | H | OH | OH |
| 7 | OH | H | H | H | H | OH | OMe |
| 8 | OH | H | H | H | H | OH | OAc |
| 9 | OH | H | H | H | H | OMe | H |
| 10 | OH | H | H | H | H | OMe | OH |
| 11 | OH | H | H | H | H | OMe | OMe |
| 12 | OH | H | H | H | H | OMe | OAc |
| 13 | OH | H | H | H | H | OEt | H |
| 14 | OH | H | H | H | H | OEt | OH |
| 15 | OH | H | H | H | H | OEt | OMe |
| 16 | OH | H | H | H | H | OEt | OAc |
| 17 | OH | H | H | H | H | O-iPr | H |
| 18 | OH | H | H | H | H | O-iPr | OH |
| 19 | OH | H | H | H | H | O-iPr | OMe |
| 20 | OH | H | H | H | H | O-iPr | OAc |
| 21 | OH | H | H | H | H | O-nPr | H |
| 22 | OH | H | H | H | H | O-nPr | OH |
| 23 | OH | H | H | H | H | O-nPr | OMe |
| 24 | OH | H | H | H | H | O-nPr | OAc |
| 25 | OH | H | H | H | H | OAc | H |
| 26 | OH | H | H | H | H | OAc | OH |
| 27 | OH | H | H | H | H | OAc | OMe |
| 28 | OH | H | H | H | H | OAc | OAc |
| 29 | OH | H | H | H | OH | H | H |
| 30 | OH | H | H | H | OH | H | OH |
| 31 | OH | H | H | H | OH | H | OMe |
| 32 | OH | H | H | H | OH | H | OAc |
| 33 | OH | H | H | H | OH | OH | H |
| 34 | OH | H | H | H | OH | OH | OH |
| 35 | OH | H | H | H | OH | OH | OMe |
| 36 | OH | H | H | H | OH | OH | OAc |
| 37 | OH | H | H | H | OH | OMe | H |
| 38 | OH | H | H | H | OH | OMe | OH |
| 39 | OH | H | H | H | OH | OMe | OMe |
| 40 | OH | H | H | H | OH | OMe | OAc |
| 41 | OH | H | H | H | OH | OEt | H |
| 42 | OH | H | H | H | OH | OEt | OH |
| 43 | OH | H | H | H | OH | OEt | OMe |
| 44 | OH | H | H | H | OH | OEt | OAc |
| 45 | OH | H | H | H | OH | O-iPr | H |
| 46 | OH | H | H | H | OH | O-iPr | OH |
| 47 | OH | H | H | H | OH | O-iPr | OMe |
| 48 | OH | H | H | H | OH | O-iPr | OAc |
| 49 | OH | H | H | H | OH | O-nPr | H |
| 50 | OH | H | H | H | OH | O-nPr | OH |
| 51 | OH | H | H | H | OH | O-nPr | OMe |
| 52 | OH | H | H | H | OH | O-nPr | OAc |
| 53 | OH | H | H | H | OH | OAc | H |
| 54 | OH | H | H | H | OH | OAc | OH |
| 55 | OH | H | H | H | OH | OAc | OMe |
| 56 | OH | H | H | H | OH | OAc | OAc |
| 57 | OH | H | H | H | OMe | H | H |
| 58 | OH | H | H | H | OMe | H | OH |
| 59 | OH | H | H | H | OMe | H | OMe |
| 60 | OH | H | H | H | OMe | H | OAc |
| 61 | OH | H | H | H | OMe | OH | H |
| 62 | OH | H | H | H | OMe | OH | OH |
| 63 | OH | H | H | H | OMe | OH | OMe |
| 64 | OH | H | H | H | OMe | OH | OAc |
| 65 | OH | H | H | H | OMe | OMe | H |
| 66 | OH | H | H | H | OMe | OMe | OH |
| 67 | OH | H | H | H | OMe | OMe | OMe |
| 68 | OH | H | H | H | OMe | OMe | OAc |
| 69 | OH | H | H | H | OMe | OEt | H |
| 70 | OH | H | H | H | OMe | OEt | OH |
| 71 | OH | H | H | H | OMe | OEt | OMe |
| 72 | OH | H | H | H | OMe | OEt | OAc |
| 73 | OH | H | H | H | OMe | O-iPr | H |
| 74 | OH | H | H | H | OMe | O-iPr | OH |
| 75 | OH | H | H | H | OMe | O-iPr | OMe |
| 76 | OH | H | H | H | OMe | O-iPr | OAc |
| 77 | OH | H | H | H | OMe | O-nPr | H |
| 78 | OH | H | H | H | OMe | O-nPr | OH |
| 79 | OH | H | H | H | OMe | O-nPr | OMe |
| 80 | OH | H | H | H | OMe | O-nPr | OAc |
| 81 | OH | H | H | H | OMe | OAc | H |
| 82 | OH | H | H | H | OMe | OAc | OH |
| 83 | OH | H | H | H | OMe | OAc | OMe |
| 84 | OH | H | H | H | OMe | OAc | OAc |
| 85 | OH | H | H | H | OAc | H | H |
| 86 | OH | H | H | H | OAc | H | OH |
| 87 | OH | H | H | H | OAc | H | OMe |
| 88 | OH | H | H | H | OAc | H | OAc |
| 89 | OH | H | H | H | OAc | OH | H |
| 90 | OH | H | H | H | OAc | OH | OH |
| 91 | OH | H | H | H | OAc | OH | OMe |
| 92 | OH | H | H | H | OAc | OH | OAc |
| 93 | OH | H | H | H | OAc | OMe | H |
| 94 | OH | H | H | H | OAc | OMe | OH |
| 95 | OH | H | H | H | OAc | OMe | OMe |
| 96 | OH | H | H | H | OAc | OMe | OAc |
| 97 | OH | H | H | H | OAc | OEt | H |
| 98 | OH | H | H | H | OAc | OEt | OH |
| 99 | OH | H | H | H | OAc | OEt | OMe |
| 100 | OH | H | H | H | OAc | OEt | OAc |
| 101 | OH | H | H | H | OAc | O-iPr | H |
| 102 | OH | H | H | H | OAc | O-iPr | OH |
| 103 | OH | H | H | H | OAc | O-iPr | OMe |
| 104 | OH | H | H | H | OAc | O-iPr | OAc |
| 105 | OH | H | H | H | OAc | O-nPr | H |
| 106 | OH | H | H | H | OAc | O-nPr | OH |
| 107 | OH | H | H | H | OAc | O-nPr | OMe |
| 108 | OH | H | H | H | OAc | O-nPr | OAc |
| 109 | OH | H | H | H | OAc | OAc | H |
| 110 | OH | H | H | H | OAc | OAc | OH |
| 111 | OH | H | H | H | OAc | OAc | OMe |
| 112 | OH | H | H | H | OAc | OAc | OAc |
| 113 | OMe | H | H | H | H | H | H |
| 114 | OMe | H | H | H | H | H | OH |
| 115 | OMe | H | H | H | H | H | OMe |
| 116 | OMe | H | H | H | H | H | OAc |
| 117 | OMe | H | H | H | H | OH | H |
| 118 | OMe | H | H | H | H | OH | OH |
| 119 | OMe | H | H | H | H | OH | OMe |
| 120 | OMe | H | H | H | H | OH | OAc |
| 121 | OMe | H | H | H | H | OMe | H |
| 122 | OMe | H | H | H | H | OMe | OH |
| 123 | OMe | H | H | H | H | OMe | OMe |
| 124 | OMe | H | H | H | H | OMe | OAc |
| 125 | OMe | H | H | H | H | OEt | H |
| 126 | OMe | H | H | H | H | OEt | OH |
| 127 | OMe | H | H | H | H | OEt | OMe |
| 128 | OMe | H | H | H | H | OEt | OAc |
| 129 | OMe | H | H | H | H | O-iPr | H |
| 130 | OMe | H | H | H | H | O-iPr | OH |
| 131 | OMe | H | H | H | H | O-iPr | OMe |
| 132 | OMe | H | H | H | H | O-iPr | OAc |
| 133 | OMe | H | H | H | H | O-nPr | H |
| 134 | OMe | H | H | H | H | O-nPr | OH |
| 135 | OMe | H | H | H | H | O-nPr | OMe |
| 136 | OMe | H | H | H | H | O-nPr | OAc |
| 137 | OMe | H | H | H | H | OAc | H |
| 138 | OMe | H | H | H | H | OAc | OH |
| 139 | OMe | H | H | H | H | OAc | OMe |
| 140 | OMe | H | H | H | H | OAc | OAc |
| 141 | OMe | H | H | H | OH | H | H |
| 142 | OMe | H | H | H | OH | H | OH |
| 143 | OMe | H | H | H | OH | H | OMe |
| 144 | OMe | H | H | H | OH | H | OAc |
| 145 | OMe | H | H | H | OH | OH | H |
| 146 | OMe | H | H | H | OH | OH | OH |
| 147 | OMe | H | H | H | OH | OH | OMe |
| 148 | OMe | H | H | H | OH | OH | OAc |
| 149 | OMe | H | H | H | OH | OMe | H |
| 150 | OMe | H | H | H | OH | OMe | OH |
| 151 | OMe | H | H | H | OH | OMe | OMe |
| 152 | OMe | H | H | H | OH | OMe | OAc |
| 153 | OMe | H | H | H | OH | OEt | H |
| 154 | OMe | H | H | H | OH | OEt | OH |
| 155 | OMe | H | H | H | OH | OEt | OMe |
| 156 | OMe | H | H | H | OH | OEt | OAc |
| 157 | OMe | H | H | H | OH | O-iPr | H |
| 158 | OMe | H | H | H | OH | O-iPr | OH |
| 159 | OMe | H | H | H | OH | O-iPr | OMe |
| 160 | OMe | H | H | H | OH | O-iPr | OAc |
| 161 | OMe | H | H | H | OH | O-nPr | H |
| 162 | OMe | H | H | H | OH | O-nPr | OH |
| 163 | OMe | H | H | H | OH | O-nPr | OMe |
| 164 | OMe | H | H | H | OH | O-nPr | OAc |
| 165 | OMe | H | H | H | OH | OAc | H |
| 166 | OMe | H | H | H | OH | OAc | OH |
| 167 | OMe | H | H | H | OH | OAc | OMe |
| 168 | OMe | H | H | H | OH | OAc | OAc |
| 169 | OMe | H | H | H | OMe | H | H |
| 170 | OMe | H | H | H | OMe | H | OH |
| 171 | OMe | H | H | H | OMe | H | OMe |
| 172 | OMe | H | H | H | OMe | H | OAc |
| 173 | OMe | H | H | H | OMe | OH | H |
| 174 | OMe | H | H | H | OMe | OH | OH |
| 175 | OMe | H | H | H | OMe | OH | OMe |
| 176 | OMe | H | H | H | OMe | OH | OAc |
| 177 | OMe | H | H | H | OMe | OMe | H |
| 178 | OMe | H | H | H | OMe | OMe | OH |
| 179 | OMe | H | H | H | OMe | OMe | OMe |
| 180 | OMe | H | H | H | OMe | OMe | OAc |
| 181 | OMe | H | H | H | OMe | OEt | H |
| 182 | OMe | H | H | H | OMe | OEt | OH |
| 183 | OMe | H | H | H | OMe | OEt | OMe |
| 184 | OMe | H | H | H | OMe | OEt | OAc |
| 185 | OMe | H | H | H | OMe | O-iPr | H |
| 186 | OMe | H | H | H | OMe | O-iPr | OH |
| 187 | OMe | H | H | H | OMe | O-iPr | OMe |
| 188 | OMe | H | H | H | OMe | O-iPr | OAc |
| 189 | OMe | H | H | H | OMe | O-nPr | H |
| 190 | OMe | H | H | H | OMe | O-nPr | OH |
| 191 | OMe | H | H | H | OMe | O-nPr | OMe |
| 192 | OMe | H | H | H | OMe | O-nPr | OAc |
| 193 | OMe | H | H | H | OMe | OAc | H |
| 194 | OMe | H | H | H | OMe | OAc | OH |
| 195 | OMe | H | H | H | OMe | OAc | OMe |
| 196 | OMe | H | H | H | OMe | OAc | OAc |
| 197 | OMe | H | H | H | OAc | H | II |
| 198 | OMe | H | H | H | OAc | H | OH |
| 199 | OMe | H | H | H | OAc | H | OMe |
| 200 | OMe | H | H | H | OAc | H | OAc |
| 201 | OMe | H | H | H | OAc | OH | H |
| 202 | OMe | H | H | H | OAc | OH | OH |
| 203 | OMe | H | H | H | OAc | OH | OMe |
| 204 | OMe | H | H | H | OAc | OH | OAc |
| 205 | OMe | H | H | H | OAc | OMe | H |
| 206 | OMe | H | H | H | OAc | OMe | OH |
| 207 | OMe | H | H | H | OAc | OMe | OMe |
| 208 | OMe | H | H | H | OAc | OMe | OAc |
| 209 | OMe | H | H | H | OAc | OEt | H |
| 210 | OMe | H | H | H | OAc | OEt | OH |
| 211 | OMe | H | H | H | OAc | OEt | OMe |
| 212 | OMe | H | H | H | OAc | OEt | OAc |
| 213 | OMe | H | H | H | OAc | O-iPr | H |
| 214 | OMe | H | H | H | OAc | O-iPr | OH |
| 215 | OMe | H | H | H | OAc | O-iPr | OMe |
| 216 | OMe | H | H | H | OAc | O-iPr | OAc |
| 217 | OMe | H | H | H | OAc | O-nPr | H |
| 218 | OMe | H | H | H | OAc | O-nPr | OH |
| 219 | OMe | H | H | H | OAc | O-nPr | OMe |
| 220 | OMe | H | H | H | OAc | O-nPr | OAc |
| 221 | OMe | H | H | H | OAc | OAc | H |
| 222 | OMe | H | H | H | OAc | OAc | OH |
| 223 | OMe | H | H | H | OAc | OAc | OMe |
| 224 | OMe | H | H | H | OAc | OAc | OAc |
| 225 | OH | H | H | OH | H | H | H |
| 226 | OH | H | H | OMe | H | H | H |
| 227 | OH | H | H | OAc | H | H | H |
| 228 | OH | H | OH | H | H | H | H |
| 229 | OH | H | OH | OH | H | H | H |
| 230 | OH | H | OH | OMe | H | H | H |
| 231 | OH | H | OH | OAc | H | H | H |
| 232 | OH | H | OMe | H | H | H | H |
| 233 | OH | H | OMe | OH | H | H | H |
| 234 | OH | H | OMe | OMe | H | H | H |
| 235 | OH | H | OMe | OAc | H | H | H |
| 236 | OH | H | OEt | H | H | H | H |
| 237 | OH | H | OEt | OH | H | H | H |
| 238 | OH | H | OEt | OMe | H | H | H |
| 239 | OH | H | OEt | OAc | H | H | H |
| 240 | OH | H | O-iPr | H | H | H | H |
| 241 | OH | H | O-iPr | OH | H | H | H |
| 242 | OH | H | O-iPr | OMe | H | H | H |
| 243 | OH | H | O-iPr | OAc | H | H | H |
| 244 | OH | H | O-nPr | H | H | H | H |
| 245 | OH | H | O-nPr | OH | H | H | H |
| 246 | OH | H | O-nPr | OMe | H | H | H |
| 247 | OH | H | O-nPr | OAc | H | H | H |
| 248 | OH | H | OAc | H | H | H | H |
| 249 | OH | H | OAc | OH | H | H | H |
| 250 | OH | H | OAc | OMe | H | H | H |
| 251 | OH | H | OAc | OAc | H | H | H |
| 252 | OH | OH | H | H | H | H | H |
| 253 | OH | OH | H | OH | H | H | H |
| 254 | OH | OH | H | OMe | H | H | H |
| 255 | OH | OH | H | OAc | H | H | H |
| 256 | OH | OH | OH | H | H | H | H |
| 257 | OH | OH | OH | OH | H | H | H |
| 258 | OH | OH | OH | OMe | H | H | H |
| 259 | OH | OH | OH | OAc | H | H | H |
| 260 | OH | OH | OMe | H | H | H | H |
| 261 | OH | OH | OMe | OH | H | H | H |
| 262 | OH | OH | OMe | OMe | H | H | H |
| 263 | OH | OH | OMe | OAc | H | H | H |
| 264 | OH | OH | OEt | H | H | H | H |
| 265 | OH | OH | OEt | OH | H | H | H |
| 266 | OH | OH | OEt | OMe | H | H | H |
| 267 | OH | OH | OEt | OAc | H | H | H |
| 268 | OH | OH | O-iPr | H | H | H | H |
| 269 | OH | OH | O-iPr | OH | H | H | H |
| 270 | OH | OH | O-iPr | OMe | H | H | H |
| 271 | OH | OH | O-iPr | OAc | H | H | H |
| 272 | OH | OH | O-nPr | H | H | H | H |
| 273 | OH | OH | O-nPr | OH | H | H | H |
| 274 | OH | OH | O-nPr | OMe | H | H | H |
| 275 | OH | OH | O-nPr | OAc | H | H | H |
| 276 | OH | OH | OAc | H | H | H | H |
| 277 | OH | OH | OAc | OH | H | H | H |
| 278 | OH | OH | OAc | OMe | H | H | H |
| 279 | OH | OH | OAc | OAc | H | H | H |
| 280 | OH | OMe | H | H | H | H | H |
| 281 | OH | OMe | H | OH | H | H | H |
| 282 | OH | OMe | H | OMe | H | H | H |
| 283 | OH | OMe | H | OAc | H | H | H |
| 284 | OH | OMe | OH | H | H | H | H |
| 285 | OH | OMe | OH | OH | H | H | H |
| 286 | OH | OMe | OH | OMe | H | H | H |
| 287 | OH | OMe | OH | OAc | H | H | H |
| 288 | OH | OMe | OMe | H | H | H | H |
| 289 | OH | OMe | OMe | OH | H | H | H |
| 290 | OH | OMe | OMe | OMe | H | H | H |
| 291 | OH | OMe | OMe | OAc | H | H | H |
| 292 | OH | OMe | OEt | H | H | H | H |
| 293 | OH | OMe | OEt | OH | H | H | H |
| 294 | OH | OMe | OEt | OMe | H | H | H |
| 295 | OH | OMe | OEt | OAc | H | H | H |
| 296 | OH | OMe | O-iPr | H | H | H | H |
| 297 | OH | OMe | O-iPr | OH | H | H | H |
| 298 | OH | OMe | O-iPr | OMe | H | H | H |
| 299 | OH | OMe | O-iPr | OAc | H | H | H |
| 300 | OH | OMe | O-nPr | H | H | H | H |
| 301 | OH | OMe | O-nPr | OH | H | H | H |
| 302 | OH | OMe | O-nPr | OMe | H | H | H |
| 303 | OH | OMe | O-nPr | OAc | H | H | H |
| 304 | OH | OMe | OAc | H | H | H | H |
| 305 | OH | OMe | OAc | OH | H | H | H |
| 306 | OH | OMe | OAc | OMe | H | H | H |
| 307 | OH | OMe | OAc | OAc | H | H | H |
| 308 | OH | OAc | H | H | H | H | H |
| 309 | OH | OAc | H | OH | H | H | H |
| 310 | OH | OAc | H | OMe | H | H | H |
| 311 | OH | OAc | H | OAc | H | H | H |
| 312 | OH | OAc | OH | H | H | H | H |
| 313 | OH | OAc | OH | OH | H | H | H |
| 314 | OH | OAc | OH | OMe | H | H | H |
| 315 | OH | OAc | OH | OAc | H | H | H |
| 316 | OH | OAc | OMe | H | H | H | H |
| 317 | OH | OAc | OMe | OH | H | H | H |
| 318 | OH | OAc | OMe | OMe | H | H | H |
| 319 | OH | OAc | OMe | OAc | H | H | H |
| 320 | OH | OAc | OEt | H | H | H | H |
| 321 | OH | OAc | OEt | OH | H | H | H |
| 322 | OH | OAc | OEt | OMe | H | H | H |
| 323 | OH | OAc | OEt | OAc | H | H | H |
| 324 | OH | OAc | O-iPr | H | H | H | H |
| 325 | OH | OAc | O-iPr | OH | H | H | H |
| 326 | OH | OAc | O-iPr | OMe | H | H | H |
| 327 | OH | OAc | O-iPr | OAc | H | H | H |
| 328 | OH | OAc | O-nPr | H | H | H | H |
| 329 | OH | OAc | O-nPr | OH | H | H | H |
| 330 | OH | OAc | O-nPr | OMe | H | H | H |
| 331 | OH | OAc | O-nPr | OAc | H | H | H |
| 332 | OH | OAc | OAc | H | H | H | H |
| 333 | OH | OAc | OAc | OH | H | H | H |
| 334 | OH | OAc | OAc | OMe | H | H | H |
| 335 | OH | OAc | OAc | OAc | H | H | H |
| 336 | OMe | H | H | H | H | H | H |
| 337 | OMe | H | H | OH | H | H | H |
| 338 | OMe | H | H | OMe | H | H | H |
| 339 | OMe | H | H | OAc | H | H | H |
| 340 | OMe | H | OH | H | H | H | H |
| 341 | OMe | H | OH | OH | H | H | H |
| 342 | OMe | H | OH | OMe | H | H | H |
| 343 | OMe | H | OH | OAc | H | H | H |
| 344 | OMe | H | OMe | H | H | H | H |
| 345 | OMe | H | OMe | OH | H | H | H |
| 346 | OMe | H | OMe | OMe | H | H | H |
| 347 | OMe | H | OMe | OAc | H | H | H |
| 348 | OMe | H | OEt | H | H | H | H |
| 349 | OMe | H | OEt | OH | H | H | H |
| 350 | OMe | H | OEt | OMe | H | H | H |
| 351 | OMe | H | OEt | OAc | H | H | H |
| 352 | OMe | H | O-iPr | H | H | H | H |
| 353 | OMe | H | O-iPr | OH | H | H | H |
| 354 | OMe | H | O-iPr | OMe | H | H | H |
| 355 | OMe | H | O-iPr | OAc | H | H | H |
| 356 | OMe | H | O-nPr | H | H | H | H |
| 357 | OMe | H | O-nPr | OH | H | H | H |
| 358 | OMe | H | O-nPr | OMe | H | H | H |
| 359 | OMe | H | O-nPr | OAc | H | H | H |
| 360 | OMe | H | OAc | H | H | H | H |
| 361 | OMe | H | OAc | OH | H | H | H |
| 362 | OMe | H | OAc | OMe | H | H | H |
| 363 | OMe | H | OAc | OAc | H | H | H |
| 364 | OMe | OH | H | H | H | H | H |
| 365 | OMe | OH | H | OH | H | H | H |
| 366 | OMe | OH | H | OMe | H | H | H |
| 367 | OMe | OH | H | OAc | H | H | H |
| 368 | OMe | OH | OH | H | H | H | H |
| 369 | OMe | OH | OH | OH | H | H | H |
| 370 | OMe | OH | OH | OMe | H | H | H |
| 371 | OMe | OH | OH | OAc | H | H | H |
| 372 | OMe | OH | OMe | H | H | H | H |
| 373 | OMe | OH | OMe | OH | H | H | H |
| 374 | OMe | OH | OMe | OMe | H | H | H |
| 375 | OMe | OH | OMe | OAc | H | H | H |
| 376 | OMe | OH | OEt | H | H | H | H |
| 377 | OMe | OH | OEt | OH | H | H | H |
| 378 | OMe | OH | OEt | OMe | H | H | H |
| 379 | OMe | OH | OEt | OAc | H | H | H |
| 380 | OMe | OH | O-iPr | H | H | H | H |
| 381 | OMe | OH | O-iPr | OH | H | H | H |
| 382 | OMe | OH | O-iPr | OMe | H | H | H |
| 383 | OMe | OH | O-iPr | OAc | H | H | H |
| 384 | OMe | OH | O-nPr | H | H | H | H |
| 385 | OMe | OH | O-nPr | OH | H | H | H |
| 386 | OMe | OH | O-nPr | OMe | H | H | H |
| 387 | OMe | OH | O-nPr | OAc | H | H | H |
| 388 | OMe | OH | OAc | H | H | H | H |
| 389 | OMe | OH | OAc | OH | H | H | H |
| 340 | OMe | OH | OAc | OMe | H | H | H |
| 341 | OMe | OH | OAc | OAc | H | H | H |
| 342 | OMe | OMe | H | H | H | H | H |
| 343 | OMe | OMe | H | OH | H | H | H |
| 344 | OMe | OMe | H | OMe | H | H | H |
| 345 | OMe | OMe | H | OAc | H | H | H |
| 346 | OMe | OMe | OH | H | H | H | H |
| 347 | OMe | OMe | OH | OH | H | H | H |
| 348 | OMe | OMe | OH | OMe | H | H | H |
| 349 | OMe | OMe | OH | OAc | H | H | H |
| 350 | OMe | OMe | OMe | H | H | H | H |
| 351 | OMe | OMe | OMe | OH | H | H | H |
| 352 | OMe | OMe | OMe | OMe | H | H | H |
| 353 | OMe | OMe | OMe | OAc | H | H | H |
| 354 | OMe | OMe | OEt | H | H | H | H |
| 355 | OMe | OMe | OEt | OH | H | H | H |
| 356 | OMe | OMe | OEt | OMe | H | H | H |
| 357 | OMe | OMe | OEt | OAc | H | H | H |
| 358 | OMe | OMe | O-iPr | H | H | H | H |
| 359 | OMe | OMe | O-iPr | OH | H | H | H |
| 360 | OMe | OMe | O-iPr | OMe | H | H | H |
| 361 | OMe | OMe | O-iPr | OAc | H | H | H |
| 362 | OMe | OMe | O-nPr | H | H | H | H |
| 363 | OMe | OMe | O-nPr | OH | H | H | H |
| 364 | OMe | OMe | O-nPr | OMe | H | H | H |
| 365 | OMe | OMe | O-nPr | OAc | H | H | H |
| 366 | OMe | OMe | OAc | H | H | H | H |
| 367 | OMe | OMe | OAc | OH | H | H | H |
| 368 | OMe | OMe | OAc | OMe | H | H | H |
| 369 | OMe | OMe | OAc | OAc | H | H | H |
| 370 | OMe | OAc | H | H | H | H | H |
| 371 | OMe | OAc | H | OH | H | H | H |
| 372 | OMe | OAc | H | OMe | H | H | H |
| 373 | OMe | OAc | H | OAc | H | H | H |
| 374 | OMe | OAc | OH | H | H | H | H |
| 375 | OMe | OAc | OH | OH | H | H | H |
| 376 | OMe | OAc | OH | OMe | H | H | H |
| 377 | OMe | OAc | OH | OAc | H | H | H |
| 378 | OMe | OAc | OMe | H | H | H | H |
| 379 | OMe | OAc | OMe | OH | H | H | H |
| 380 | OMe | OAc | OMe | OMe | H | H | H |
| 381 | OMe | OAc | OMe | OAc | H | H | H |
| 382 | OMe | OAc | OEt | H | H | H | H |
| 383 | OMe | OAc | OEt | OH | H | H | H |
| 384 | OMe | OAc | OEt | OMe | H | H | H |
| 385 | OMe | OAc | OEt | OAc | H | H | H |
| 386 | OMe | OAc | O-iPr | H | H | H | H |
| 387 | OMe | OAc | O-iPr | OH | H | H | H |
| 388 | OMe | OAc | O-iPr | OMe | H | H | H |
| 389 | OMe | OAc | O-iPr | OAc | H | H | H |
| 390 | OMe | OAc | O-nPr | H | H | H | H |
| 391 | OMe | OAc | O-nPr | OH | H | H | H |
| 392 | OMe | OAc | O-nPr | OMe | H | H | H |
| 393 | OMe | OAc | O-nPr | OAc | H | H | H |
| 394 | OMe | OAc | OAc | H | H | H | H |
| 395 | OMe | OAc | OAc | OH | H | H | H |
| 396 | OMe | OAc | OAc | OMe | H | H | H |
| 397 | OMe | OAc | OAc | OAc | H | H | H |
| 398 | OH | H | H | OH | H | H | OH |
| 399 | OH | H | H | OMe | H | H | OMe |
| 400 | OH | H | H | OAc | H | H | OAc |
| 401 | OH | H | OH | H | H | OH | H |
| 402 | OH | H | OH | OH | H | OH | OH |
| 403 | OH | H | OH | OMe | H | OH | OMe |
| 404 | OH | H | OH | OAc | H | OH | OAc |
| 405 | OH | H | OMe | H | H | OMe | H |
| 406 | OH | H | OMe | OH | H | OMe | OH |
| 407 | OH | H | OMe | OMe | H | OMe | OMe |
| 408 | OH | H | OMe | OAc | H | OMe | OAc |
| 409 | OH | H | OEt | H | H | OEt | H |
| 410 | OH | H | OEt | OH | H | OEt | OH |
| 411 | OH | H | OEt | OMe | H | OEt | OMe |
| 412 | OH | H | OEt | OAc | H | CEt | OAc |
| 413 | OH | H | O-iPr | H | H | O-iPr | H |
| 414 | OH | H | O-iPr | OH | H | O-iPr | OH |
| 415 | OH | H | O-iPr | OMe | H | O-iPr | OMe |
| 416 | OH | H | O-iPr | OAc | H | O-iPr | OAc |
| 417 | OH | H | O-nPr | H | H | O-nPr | H |
| 418 | OH | H | O-nPr | OH | H | O-nPr | OH |
| 419 | OH | H | O-nPr | OMe | H | O-nPr | OMe |
| 420 | OH | H | O-nPr | OAc | H | O-nPr | OAc |
| 421 | OH | H | OAc | H | H | OAc | H |
| 422 | OH | H | OAc | OH | H | OAc | OH |
| 423 | OH | H | OAc | OMe | H | OAc | OMe |
| 424 | OH | H | OAc | OAc | H | OAc | OAc |
| 425 | OH | OH | H | H | OH | H | H |
| 426 | OH | OH | H | OH | OH | H | OH |
| 427 | OH | OH | H | OMe | OH | H | OMe |
| 428 | OH | OH | H | OAc | OH | H | OAc |
| 429 | OH | OH | OH | H | OH | OH | H |
| 430 | OH | OH | OH | OH | OH | OH | OH |
| 431 | OH | OH | OH | OMe | OH | OH | OMe |
| 432 | OH | OH | OH | OAc | OH | OH | OAc |
| 433 | OH | OH | OMe | H | OH | OMe | H |
| 444 | OH | OH | OMe | OH | OH | OMe | OH |
| 445 | OH | OH | OMe | OMe | OH | OMe | OMe |
| 446 | OH | OH | OMe | OAc | OH | OMe | OAc |
| 447 | OH | OH | OEt | H | OH | OEt | H |
| 448 | OH | OH | OEt | OH | OH | OEt | OH |
| 449 | OH | OH | OEt | OMe | OH | OEt | OMe |
| 450 | OH | OH | OEt | OAc | OH | OEt | OAc |
| 451 | OH | OH | O-iPr | H | OH | O-iPr | H |
| 452 | OH | OH | O-iPr | OH | OH | O-iPr | OH |
| 453 | OH | OH | O-iPr | OMe | OH | O-iPr | OMe |
| 454 | OH | OH | O-iPr | OAc | OH | O-iPr | OAc |
| 455 | OH | OH | O-nPr | H | OH | O-nPr | H |
| 456 | OH | OH | O-nPr | OH | OH | O-nPr | OH |
| 457 | OH | OH | O-nPr | OMe | OH | O-nPr | OMe |
| 458 | OH | OH | O-nPr | OAc | OH | O-nPr | OAc |
| 459 | OH | OH | OAc | H | OH | OAc | H |
| 460 | OH | OH | OAc | OH | OH | OAc | OH |
| 461 | OH | OH | OAc | OMe | OH | OAc | OMe |
| 462 | OH | OH | OAc | OAc | OH | OAc | OAc |
| 463 | OH | OMe | H | H | OMe | H | H |
| 464 | OH | OMe | H | OH | OMe | H | OH |
| 465 | OH | OMe | H | OMe | OMe | H | OMe |
| 466 | OH | OMe | H | OAc | OMe | H | OAc |
| 467 | OH | OMe | OH | H | OMe | OH | H |
| 468 | OH | OMe | OH | OH | OMe | OH | OH |
| 469 | OH | OMe | OH | OMe | OMe | OH | OMe |
| 470 | OH | OMe | OH | OAc | OMe | OH | OAc |
| 471 | OH | OMe | OMe | H | OMe | OMe | H |
| 472 | OH | OMe | OMe | OH | OMe | OMe | OH |
| 473 | OH | OMe | OMe | OMe | OMe | OMe | OMe |
| 474 | OH | OMe | OMe | OAc | OMe | OMe | OAc |
| 475 | OH | OMe | OEt | H | OMe | OEt | H |
| 476 | OH | OMe | OEt | OH | OMe | OEt | OH |
| 477 | OH | OMe | OEt | OMe | OMe | OEt | OMe |
| 478 | OH | OMe | OEt | OAc | OMe | OEt | OAc |
| 479 | OH | OMe | O-iPr | H | OMe | O-iPr | H |
| 480 | OH | OMe | O-iPr | OH | OMe | O-iPr | OH |
| 481 | OH | OMe | O-iPr | OMe | OMe | O-iPr | OMe |
| 482 | OH | OMe | O-iPr | OAc | OMe | O-iPr | OAc |
| 483 | OH | OMe | O-nPr | H | OMe | O-nPr | H |
| 484 | OH | OMe | O-nPr | OH | OMe | O-nPr | OH |
| 485 | OH | OMe | O-nPr | OMe | OMe | O-nPr | OMe |
| 486 | OH | OMe | O-nPr | OAc | OMe | O-nPr | OAc |
| 487 | OH | OMe | OAc | H | OMe | OAc | H |
| 488 | OH | OMe | OAc | OH | OMe | OAc | OH |
| 489 | OH | OMe | OAc | OMe | OMe | OAc | OMe |
| 490 | OH | OMe | OAc | OAc | OMe | - OAc | OAc |
| 491 | OH | OAc | H | H | OAc | H | H |
| 492 | OH | OAc | H | OH | OAc | H | OH |
| 493 | OH | OAc | H | OMe | OAc | H | OMe |
| 494 | OH | OAc | H | OAc | OAc | H | OAc |
| 495 | OH | OAc | OH | H | OAc | OH | H |
| 496 | OH | OAc | OH | OH | OAc | OH | OH |
| 497 | OH | OAc | OH | OMe | OAc | OH | OMe |
| 498 | OH | OAc | OH | OAc | OAc | OH | OAc |
| 499 | OH | OAc | OMe | H | OAc | OMe | H |
| 500 | OH | OAc | OMe | OH | OAc | OMe | OH |
| 501 | OH | OAc | OMe | OMe | OAc | OMe | OMe |
| 502 | OH | OAc | OMe | OAc | OAc | OMe | OAc |
| 503 | OH | OAc | OEt | H | OAc | OEt | H |
| 504 | OH | OAc | OEt | OH | OAc | OEt | OH |
| 505 | OH | OAc | OEt | OMe | OAc | OEt | OMe |
| 506 | OH | OAc | OEt | OAc | OAc | OEt | OAc |
| 507 | OH | OAc | O-iPr | H | OAc | O-iPr | H |
| 508 | OH | OAc | O-iPr | OH | OAc | O-iPr | OH |
| 509 | OH | OAc | O-iPr | OMe | OAc | O-iPr | OMe |
| 510 | OH | OAc | O-iPr | OAc | OAc | O-iPr | OAc |
| 511 | OH | OAc | O-nPr | H | OAc | O-nPr | H |
| 512 | OH | OAc | O-nPr | OH | OAc | O-nPr | OH |
| 513 | OH | OAc | O-nPr | OMe | OAc | O-nPr | OMe |
| 514 | OH | OAc | O-nPr | OAc | OAc | O-nPr | OAc |
| 515 | OH | OAc | OAc | H | OAc | OAc | H |
| 516 | OH | OAc | OAc | OH | OAc | OAc | OH |
| 517 | OH | OAc | OAc | OMe | OAc | OAc | OMe |
| 518 | OH | OAc | OAc | OAc | OAc | OAc | OAc |
| 519 | OMe | H | H | H | H | H | H |
| 520 | OMe | H | H | OH | H | H | OH |
| 521 | OMe | H | H | OMe | H | H | OMe |
| 522 | OMe | H | H | OAc | H | H | OAc |
| 523 | OMe | H | OH | H | H | OH | H |
| 524 | OMe | H | OH | OH | H | OH | OH |
| 525 | OMe | H | OH | OMe | H | OH | OMe |
| 526 | OMe | H | OH | OAc | H | OH | OAc |
| 527 | OMe | H | OMe | H | H | OMe | H |
| 528 | OMe | H | OMe | OH | H | OMe | OH |
| 529 | OMe | H | OMe | OMe | H | OMe | OMe |
| 530 | OMe | H | OMe | OAc | H | OMe | CAc |
| 531 | OMe | H | OEt | H | H | OEt | H |
| 532 | OMe | H | OEt | OH | H | OEt | OH |
| 533 | OMe | H | OEt | OMe | H | OEt | OMe |
| 534 | OMe | H | OEt | OAc | H | OEt | OAc |
| 535 | OMe | H | O-iPr | H | H | O-iPr | H |
| 536 | OMe | H | O-iPr | OH | H | O-iPr | OH |
| 537 | OMe | H | O-iPr | OMe | H | O-iPr | OMe |
| 538 | OMe | H | O-iPr | OAc | H | O-iPr | OAc |
| 539 | OMe | H | O-nPr | H | H | O-nPr | H |
| 540 | OMe | H | O-nPr | OH | H | O-nPr | OH |
| 541 | OMe | H | O-nPr | OMe | H | O-nPr | OMe |
| 542 | OMe | H | O-nPr | OAc | H | O-nPr | OAc |
| 543 | OMe | H | OAc | H | H | OAc | H |
| 544 | OMe | H | OAc | OH | H | OAc | OH |
| 545 | OMe | H | OAc | OMe | H | OAc | OMe |
| 546 | OMe | H | OAc | OAc | H | OAc | OAc |
| 547 | OMe | OH | H | H | OH | H | H |
| 548 | OMe | OH | H | OH | OH | H | OH |
| 549 | OMe | OH | H | OMe | OH | H | OMe |
| 550 | OMe | OH | H | OAc | OH | H | OAc |
| 551 | OMe | OH | OH | H | OH | OH | H |
| 552 | OMe | OH | OH | OH | OH | OH | OH |
| 553 | OMe | OH | OH | OMe | OH | OH | OMe |
| 554 | OMe | OH | OH | OAc | OH | OH | OAc |
| 555 | OMe | OH | OMe | H | OH | OMe | H |
| 556 | OMe | OH | OMe | OH | OH | OMe | OH |
| 557 | OMe | OH | OMe | OMe | OH | OMe | OMe |
| 558 | OMe | OH | OMe | OAc | OH | OMe | OAc |
| 559 | OMe | OH | OEt | H | OH | OEt | H |
| 560 | OMe | OH | OEt | OH | OH | OEt | OH |
| 561 | OMe | OH | OEt | OMe | OH | OEt | OMe |
| 562 | OMe | OH | OEt | OAc | OH | OEt | OAc |
| 563 | OMe | OH | O-iPr | H | OH | O-iPr | H |
| 564 | OMe | OH | O-iPr | OH | OH | O-iPr | OH |
| 565 | OMe | OH | O-iPr | OMe | OH | O-iPr | OMe |
| 566 | OMe | OH | O-iPr | OAc | OH | O-iPr | OAc |
| 567 | OMe | OH | O-nPr | H | OH | O-nPr | H |
| 568 | OMe | OH | O-nPr | OH | OH | O-nPr | OH |
| 569 | OMe | OH | O-nPr | OMe | OH | O-nPr | OMe |
| 570 | OMe | OH | O-nPr | OAc | OH | O-nPr | OAc |
| 571 | OMe | OH | OAc | H | OH | OAc | H |
| 572 | OMe | OH | OAc | OH | OH | OAc | OH |
| 573 | OMe | OH | OAc | OMe | OH | OAc | OMe |
| 574 | OMe | OH | OAc | OAc | OH | OAc | OAc |
| 575 | OMe | OMe | H | H | OMe | H | H |
| 576 | OMe | OMe | H | OH | OMe | H | OH |
| 577 | OMe | OMe | H | OMe | OMe | H | OMe |
| 578 | OMe | OMe | H | OAc | OMe | H | OAc |
| 579 | OMe | OMe | OH | H | OMe | OH | H |
| 580 | OMe | OMe | OH | OH | OMe | OH | OH |
| 581 | OMe | OMe | OH | OMe | OMe | OH | OMe |
| 582 | OMe | OMe | OH | OAc | OMe | OH | OAc |
| 583 | OMe | OMe | OMe | H | OMe | OMe | H |
| 584 | OMe | OMe | OMe | OH | OMe | OMe | OH |
| 585 | OMe | OMe | OMe | OMe | OMe | OMe | OMe |
| 586 | OMe | OMe | OMe | OAc | OMe | OMe | OAc |
| 587 | OMe | OMe | OEt | H | OMe | OEt | H |
| 588 | OMe | OMe | OEt | OH | OMe | OEt | OH |
| 589 | OMe | OMe | OEt | OMe | OMe | OEt | OMe |
| 590 | OMe | OMe | OEt | OAc | OMe | OEt | OAc |
| 591 | OMe | OMe | O-iPr | H | OMe | O-iPr | H |
| 592 | OMe | OMe | O-iPr | OH | OMe | O-iPr | OH |
| 593 | OMe | OMe | O-iPr | OMe | OMe | O-iPr | OMe |
| 594 | OMe | OMe | O-iPr | OAc | OMe | O-iPr | OAc |
| 595 | OMe | OMe | O-nPr | H | OMe | O-nPr | H |
| 596 | OMe | OMe | O-nPr | OH | OMe | O-nPr | OH |
| 597 | OMe | OMe | O-nPr | OMe | OMe | O-nPr | OMe |
| 598 | OMe | OMe | O-nPr | OAc | OMe | O-nPr | OAc |
| 599 | OMe | OMe | OAc | H | OMe | OAc | H |
| 600 | OMe | OMe | OAc | OH | OMe | OAc | OH |
| 601 | OMe | OMe | OAc | OMe | OMe | OAc | OMe |
| 602 | OMe | OMe | OAc | OAc | OMe | OAc | OAc |
| 603 | OMe | OAc | H | H | OAc | H | H |
| 604 | OMe | OAc | H | OH | OAc | H | OH |
| 605 | OMe | OAc | H | OMe | OAc | H | OMe |
| 606 | OMe | OAc | H | OAc | OAc | H | OAc |
| 607 | OMe | OAc | OH | H | OAc | OH | H |
| 608 | OMe | OAc | OH | OH | OAc | OH | OH |
| 609 | OMe | OAc | OH | OMe | OAc | OH | OMe |
| 610 | OMe | OAc | OH | OAc | OAc | OH | OAc |
| 611 | OMe | OAc | OMe | H | OAc | OMe | H |
| 612 | OMe | OAc | OMe | OH | OAc | OMe | OH |
| 613 | OMe | OAc | OMe | OMe | OAc | OMe | OMe |
| 614 | OMe | OAc | OMe | OAc | OAc | OMe | OAc |
| 615 | OMe | OAc | OEt | H | OAc | OEt | H |
| 616 | OMe | OAc | OEt | OH | OAc | OEt | OH |
| 617 | OMe | OAc | OEt | OMe | OAc | OEt | OMe |
| 618 | OMe | OAc | OEt | OAc | OAc | OEt | OAc |
| 619 | OMe | OAc | O-iPr | H | OAc | O-iPr | H |
| 620 | OMe | OAc | O-iPr | OH | OAc | O-iPr | OH |
| 621 | OMe | OAc | O-iPr | OMe | OAc | O-iPr | OMe |
| 622 | OMe | OAc | O-iPr | OAc | OAc | O-iPr | OAc |
| 623 | OMe | OAc | O-nPr | H | OAc | O-nPr | H |
| 624 | OMe | OAc | O-nPr | OH | OAc | O-nPr | OH |
| 625 | OMe | OAc | O-nPr | OMe | OAc | O-nPr | OMe |
| 626 | OMe | OAc | O-nPr | OAc | OAc | O-nPr | OAc |
| 627 | OMe | OAc | OAc | H | OAc | OAc | H |
| 628 | OMe | OAc | OAc | OH | OAc | OAc | OH |
| 629 | OMe | OAc | OAc | OMe | OAc | OAc | OMe |
| 630 | OMe | OAc | OAc | OAc | OAc | OAc | OAc |

Residues not specifically listed in the table above are understood to be hydrogen, unless specified otherwise.

It should be appreciated that certain compounds of the invention may contain one or more chiral atoms. Thus, the invention encompasses all stereoisomers, including enatiomers, diastereoisomers and mixtures thereof. In a one embodiment, the invention includes the racemic or either the R- or S-enantiomers of all the compounds described herein. The enantiomers may each be provided in a form substantially free of the other enantiomer (e.g., at least 75% free (w/w), at least 90% free (w/w) or at least 99% free (w/w)) or as mixtures (e.g., racemic mixtures).

Preferred compounds of the invention are 5-hydroxy-7-(4″-hydroxy-3″methoxyphenyl)-1-phenyl-3-heptanone (compound Lima-10 or AO-1), 5-methoxy-7-(4″-hydroxy-3″methoxyphenyl)-1-phenyl-3-heptanone (compound IIIa-122 or AO-2), 7-(4″-hydroxyphenyl)-1-phenyl-hept-4-en-3-one (compound IIIb-2 or AO-3), 7-(4″-hydroxy-3″-methoxyphenyl)-1-phenyl-hept-4-en-3-one (compound IIIb-10 or AO-4), and 1,7-diphenylhept-4-en-3-one (compound IIIb-1 or AO-5).

In certain embodiments of the invention, the compositions of the invention do not comprise any one or more of the compounds listed above, i.e., compounds IIIa-I through to III-630, compounds IIIb-1 through to IIIb-630.

The present invention also provides compositions comprising one or more compounds of the invention. For example, in one embodiment, a composition of the invention comprises two, three, four, five, six, seven, eight, nine, or ten or more compounds of the invention. In general, the composition is not a natural source of such compounds. Examples of a natural source of such compounds include the Alpina officinarum plant, a part of the Alpina officinarum plant, such as the rhizome, and other closely related Alpina species and their anatomical parts. Other natural sources of such compounds include Alpinia species, such as A. katsumadai, A. oxyphlla, A. conchigera, A. blepharocalyx, and Zingiber officinale. The term “natural source” as used herein is not limited to a plant or its anatomical part in its natural form, but is intended to include compositions or extracts which have been prepared from the plant or its parts by a process that does not selectively remove or retain one or more particular diarylheptanoids relative to the other diarylheptanoids, for example, juice that is mechanically extracted from the rhizome, mechanically disrupted materials of the Alpina officinarum plant or its parts, and powdered roots of the Alpina officinarum plant.

In one embodiment, the diarylheptanoid in the composition is isolated.

In one aspect, a composition of the invention comprises a mixture of diarylheptanoids, including one or more of the compounds of formula Ia and/or formula Ib, or a pharmaceutically acceptable salt, solvate or hydrate thereof, wherein (i) the concentration of a diarylheptanoid in the composition is different from that of a natural source of the diarylhaptanopid; and/or that (ii) the ratio of the concentration of one diarylheptanoid in the composition to that of another diarylheptanoid is different from that found in a natural source of the diarylhaptanoids. For example, a two-fold increase or decrease in concentration of one of the diarylhaptanods can be used to distinguish a composition of the invention from a natural source.

Such a composition can be prepared, for example, by processing a natural source of diarylheptanoids such that at least one particular diarylheptanoid has been selectively removed or enriched or retained. Alternatively, one or more purified diarylheptanoids can be used to make such compositions. Such a composition can also be prepared, for example, by adding an amount of at least one diarylheptanoid to a natural source or prepared natural source of the diarylheptanoids.

Methods for synthesizing diarylheptanoids are known in the art and are discussed in details in Section 5.2 hereinbelow, and an exemplary method for extracting diarylheptanoids of the invention is provided in Section 6.

As the diarylheptanoids of the invention can be used in food compositions, one method for selectively removing, enriching or retaining diarylheptanoids is supercritical fluid extraction. This technique, which generally utilizes carbon dioxide, is known in the art, especially for preparing food and medicinal substances for human consumption. See, for example, Hamburger et al., Phytochemical Analysis (2004), 15(1), 46-54; Simandi et al., Recents Progres en Genie des Procedes (1999) 13(71), 157-164, the disclosures of which are incorporated herein by reference in their entirety. A co-solvent, such as ethanol, may also be used in the technique. Accordingly, in one embodiment, the invention encompasses compositions comprising one or more diarylheptanoids of the invention that have been obtained via supercritical carbon dioxide extraction from a natural source of the diarylheptanoids. Such compositions are produced by a process comprising treating a natural source of diarylheptanoids, such as an extract of or a rhizome of Alpinia officinarum, with supercritical carbon dioxide for a period of time and at a pressure and/or a temperature that extract diarylheptanoids, including conditions that selectively extract specific diarylheptanoids of the invention; and collecting the extracted diarylheptanoids.

In specific embodiments, a composition of the invention can comprise a mixture of compounds having the formula Ia and/or formula Ib, or a pharmaceutically acceptable salt, solvate or hydrate thereof, wherein (i) the concentration of one or more of the compounds having the formula Ia is increased or decreased relative to that found in a natural source of the compounds; (ii) the concentration of one or more of the compounds having the formula Ib is increased or decreased relative to that found in a natural source of the compounds; (iii) the concentration of one or more of the compounds having the formula IIa is increased or decreased relative to that found in a natural source of the compounds; (iv) the concentration of one or more of the compounds having the formula IIb is increased or decreased relative to that found in a natural source of the compounds; (v) the concentration of one or more of the compounds having the formula IIIa or IIIb is increased or decreased relative to that found in a natural source of the compounds; (vi) the concentration of 5-hydroxy-7-(4″-hydroxy-3″methoxyphenyl)-1-phenyl-3-heptanone is greater than about 134 μg/g; is at least about 200 μg/g, at least about 500 μg/g, at least about 1 mg/g, at least about 2 mg/g, at least about 5 mg/g, at least 10 about mg/g, at least about 100 mg/g; or is less than about 100 μg/g, less than about 50 μg/g, less than about 20 μg/g, less than about 10 μg/g; (vii) the concentration of 5-methoxy-7-(4″-hydroxy-3″methoxyphenyl)-1-phenyl-3-heptanone is greater than about 40 μg/g; is at least about 100 μg/g, at least about 200 μg/g, at least about 500 μg/g, at least about 1 mg/g, at least about 2 mg/g, at least about 5 mg/g, at least about 10 mg/g, at least about 100 mg/g; or is less than about 20 μg/g, less than about 10 μg/g, less than about 5 μg/g; (viii) the concentration of 7-(4″-hydroxyphenyl)-1-phenyl-hept-4-en-3-one is greater than about 74 μg/g; is at least about 100 μg/g, at least about 200 μg/g, at least about 500 μg/g, at least about 1 mg/g, at least about 2 mg/g, at least about 5 mg/g, at least about 10 mg/g, at least about 100 mg/g; or is less than about 50 μg/g, less than about 20 μg/g, less than about 10 μg/g; (ix) the concentration of 7-(4″-hydroxy-3″-methoxyphenyl)-1-phenyl-hept-4-en-3-one is greater than about 168 μg/g; is at least about 200 μg/g, at least about 500 μg/g, at least about 1 mg/g, at least about 2 mg/g, at least about 5 mg/g, at least about 10 mg/g, at least 100 about mg/g; or is less than about 100 μg/g, less than about 50 μg/g, less than about 20 [μg/g, less than about 10 μg/g; (ix) the concentration of 1,7-diphenylhept-4-en-3-one is greater than about 138 μg/g; is at least about 200 μg/g, at least about 500 μg/g, at least about 1 mg/g, at least about 2 mg/g, at least about 5 mg/g, at least about 10 mg/g, at least about 100 mg/g; or is less than about 100 μg/g, less than about 50 μg/g, less than about 20 μg/g, less than about 10 μg/g; and/or (x) the concentration of one or more of the compounds having the formula Ia and/or the formula IIb is present at a concentration greater than about 500 μg/g, about 1 mg/g, about 2 mg/g, about 5 mg/g, about 10 mg/g, or about 100 mg/g of the composition.

In yet another embodiment, the invention provides a composition comprising a mixture of compounds having the formula Ia, formula Ib, formula Ia, and/or formula IIb, or a pharmaceutically acceptable salt, solvate or hydrate thereof, including but not limited to the compounds of formula IIIa and IIIb, or a pharmaceutically acceptable salt, solvate or hydrate thereof, wherein the percentages (by dry weight) of one or more diarylheptanoids relative to the total content of diarylheptanoids is different from that in a natural source of the diarylheptanoids. In one embodiment, the total diarylheptanoids in a composition is the total of compounds having the formula Ia and formula lb. In a preferred embodiment, a composition comprises 5-hydroxy-7-(4″-hydroxy-3″methoxyphenyl)-1-phenyl-3-heptanone which constitutes at least about 25%, at least about 35%, at least about 50%, at least about 75%, at least about 80%, or at least about 90% of the total diarylheptanoids in the composition. In another embodiment, a composition comprises 5-methoxy-7-(4″-hydroxy-3″methoxyphenyl)-1-phenyl-3-heptanone which constitutes at least about 10%, at least about 20%, at least about 25%, at least about 35%, at least about 50%, at least about 75%, at least about 80%, or at least about 90% of the total diarylheptanoids in the composition. In another embodiment, a composition comprises 7-(4″-hydroxyphenyl)-1-phenyl-hept-4-en-3-one which constitutes at least about 15%, at least about 20%, at least about 25%, at least about 35%, at least about 50%, at least about 75%, at least about 80%, or at least about 90% of the total diarylheptanoids in the composition. In another embodiment, a composition comprises 7-(4″-hydroxy-3″-methoxyphenyl)-1-phenyl-hept-4-en-3-one which constitutes at least about 35%, at least about 40%, at least about 50%, at least about 60%, at least about 70%, at least about 75%, at least about 80%, or at least about 90% of the total diarylheptanoids in the composition. In another embodiment, a composition comprises 1,7-diphenylhept-4-en-3-one which constitutes at least about 30%, at least about 35%, at least about 50%, at least about 75%, at least about 80%, or at least about 90% of the total diarylheptanoids in the composition.

In another embodiment, a composition of the invention comprises a mixture of compounds having the formula Ia, formula Ib, formula Ia, and/or formula IIb or a pharmaceutically acceptable salt, solvate or hydrate thereof, including but not limited to the compounds of formula IIIa and IIIb, or a pharmaceutically acceptable salt, solvate or hydrate thereof, wherein the ratio of certain diarylheptanoids in the composition is different from that found in a natural source of the compounds. In a preferred embodiment, the ratios of five compounds, 5-hydroxy-7-(4″-hydroxy-3″methoxyphenyl)-1-phenyl-3-heptanone: 5-methoxy-7-(4″-hydroxy-3″methoxyphenyl)-1-phenyl-3-heptanone: 7-(4″-hydroxyphenyl)-1-phenyl-hept-4-en-3-one: 7-(4″-hydroxy-3″-methoxyphenyl)-1-phenyl-hept-4-en-3-one: 1,7-diphenylhept-4-en-3-one, relative to each other in a composition is not 3:1:2:4:3, respectively. In various embodiments, the composition of the invention comprising a mixture of diaryiheptanoids can be characterized by the ratio of two or more specific diarylheptanoids present in the mixture, which can be represented by the ratios r1:r2, r1:r2:r3, r1:r2:r3:r4, r1:r2:r3:r4:r5, r1:r2:r3:r4:r5:r6 . . . rx, where x is the number of specific diarylheptanoids that have been quantified in the mixture. To distinguish different mixtures or compositions, not all diarylheptanoids need to be quantified as long as there is at least one difference in the ratio of at least two diarylheptanoid compounds relative to that found in a natural source of the compounds. The ratio can be expressed as a quotient of the concentrations or amounts of the respective compounds in the mixtures. Accordingly, the compositions of the invention can be described by stating that the ratio of two or more specific diarylheptanoid compounds is greater than, equal to, or less than a ratio or a specified set of ratios. The ratio of two diarylheptanoids in a composition of the invention can also be represented by the expression p:q, where p and q are integers, e.g., 1: 1, 2:1, 3: 1, 4:1, 5:1, 10:1, 15:1, 20:1, 50:1, 75:1, 100:1, 200:1, 500:1, 1000:1. For example, a composition of the invention can comprise more than one compound having the formula Ia or the formula Ib, wherein the ratio of a first compound having the formula IIb relative to a second compound having the formula Ia or the formula Ib but not the formula IIb is greater than, equal to, or less than a ratio represented by the expression p:q.

In one embodiment, a composition of the invention comprises a diarylheptanoid compound or a compound having the formula Ia or Ib, wherein the compound is not a specific, isolated diarylheptanoid compound described in any one of the following references: Kiuchi et al., 1992, Chemical Pharmaceutical Bulletin, 40(2) 387-91; Hikino et al., 1985, J. Ethnopharmacology, 14:31-39; Itokawa et al., Chemical Pharmaceutical Bulletin, (1981) 29:2383-2385; Shin et al., J. Nat. Prod. (2002) 65:1315-1318; Bu et al., Zhongshan Daxue Xuebao, Ziran Kexueban (2000) 39(2):41-45; Uehara et al., Chemical Pharmaceutical Bulletin, (1987) 35:3298-3304; Itokawa et al., Chemical Pharmaceutical Bulletin, (1985) 33(11):4889-4893; Shen et al., Tianran Chanwu Yanjiu Yu Kaifa (1998) 10:33-36; Japanese patent applications JP 62099325 by Miyahara et al.; JP 05000937 and JP 3059423 by Yamahara; JP 59098026 and JP 02039494, which are incorporated herein by reference in their entirety.

### 5.2 Methods for Making the Compounds of the Invention

The diarylheptanoid compounds of the invention can be prepared from readily available starting materials using the following exemplary general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Third Edition, Wiley, New York, 1999, and references cited therein.

Exemplary methods of synthesizing diarylheptanoid compounds are described in detail in Zhu et al., 2000, Organic Preparation and Procedures Int. 32:505-546, Huang et al., 1998, Chem. J. Chin. Univ. 19:78, Kato et al., 1984, Chem. Pharm. Bull. 32:3323, Semmelhack et al., 1975, J. Am. Chem. Soc. 97:3873, and Semmelhack et al., 1981, J. Am. Chem. Soc. 103:6460, the contents of each are incorporated herein by reference in their entireties.

In exemplary methods of synthesis, the diarylheptanoid compounds of the invention according to formulas Ia, Ia and IIIa can be prepared according to the method of Kato et al. or according to the methods of Semmelhack et al:

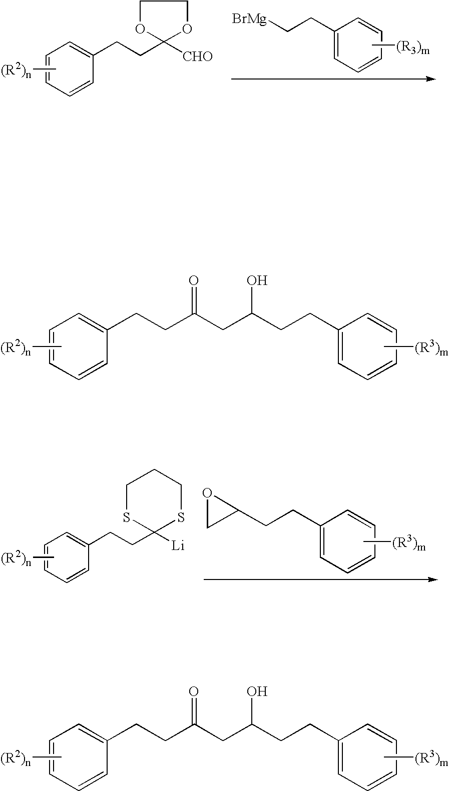

In further exemplary methods of synthesis, the diarylheptanoid compounds of the invention according to formulas Ib, IIb and IIIb can be prepared by coupling an aldehyde with a ketone according to the method of Huang et al.:

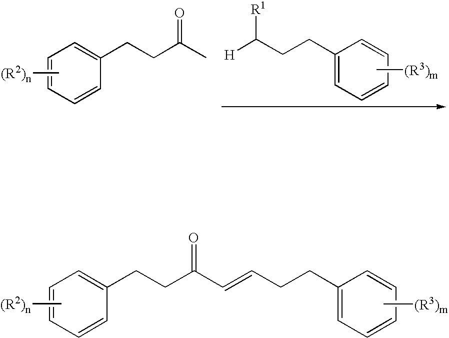

The ketones and aldehydes employed in the above-described coupling reactions are either known or can be prepared from known compounds by conventional procedures and/or according to procedures described in the references discussed above.

Once synthesized, a compound of the invention can be isolated from chemical precursors or other chemicals using standard purification techniques such as, for example, chromatography (e.g., flash column chromatography and HPLC), asymmetric methods of synthesis, recrystallization and differential solubility.

### 5.3 Agents Useful In Combination With the Compounds of the Invention

The present invention provides methods for preventing, managing, treating, or ameliorating disorders (e.g., proliferative disorders or inflammatory disorders) comprising administering to a subject in need thereof a composition of the invention, or one or more compounds of the invention and one or more therapies (e.g., one or more prophylactic or therapeutic agents) other than compounds of the invention. The present invention also provides compositions comprising one or more compounds of the invention and one or more prophylactic or therapeutic agents in addition to compounds of the invention and methods of preventing, managing, treating, or ameliorating a proliferative disorder or an inflammatory disorder utilizing said compositions. Therapeutic or prophylactic agents include, but are not limited to, plant extracts, small molecules, synthetic drugs, peptides, polypeptides, proteins, nucleic acids (e.g., DNA and RNA nucleotides including, but not limited to, antisense nucleotide sequences, RNAi, triple helices and nucleotide sequences encoding biologically active proteins, polypeptides or peptides), antibodies, synthetic or natural inorganic molecules, mimetic agents, and synthetic or natural organic molecules.

Any agent which contributes to the prevention, management, treatment, or amelioration of a disorder (e.g., a proliferative disorder or an inflammatory disorder) or one or more symptoms thereof can be used in combination with a compound of the invention in accordance with the invention described herein. See, e.g., Gilman et al., Goodman and Gilman': The Pharmacological Basis of Therapeutics, Tenth Ed., McGraw-Hill, New York, 2001; The Merck Manual of Diagnosis and Therapy, Berkow, M. D. et al. (eds.), 17th Ed., Merck Sharp & Dohme Research Laboratories, Rahway, N.J., 1999; Cecil Textbook of Medicine, 20th Ed., Bennett and Plum (eds.), W. B. Saunders, Philadelphia, 1996 for information regarding prophylactic or therapeutic agents which have been or are currently being used for preventing, treating, managing, or ameliorating proliferative disorders or inflammatory disorders or one or more symptoms thereof. Examples of such agents include, but are not limited to, anti-inflammatory agents (e.g., corticosteroids (e.g., prednisone and hydrocortisone), glucocorticoids, steroids, non-steriodal anti-inflammatory drugs (e.g., aspirin, ibuprofen, diclofenac, and COX-2 inhibitors), beta-agonists, anticholinergic agents and methyl xanthines), immunomodulatory agents, gold injections, sulphasalazine, penicillamine, anti-angiogenic agents (e.g., angiostatin, TNF-α antagonists (e.g., anti-TNFα antibodies), and endostatin), anti-fibrotics, antiemetic agents (e.g., metoclopromide, domperidone, prochlorperazine, promethazine, chlorpromazine, trimethobenzamide, ondansetron, granisetron, hydroxyzine, acethylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinols, thiethylperazine, thioproperazine and tropisetron), opioids (e.g., morphine, heroin, hydromorphone, hydrocodone, oxymorphone, oxycodone, metopon, apomorphine, normorphine, etorphine, buprenorphine, meperidine, lopermide, anileridine, ethoheptazine, piminidine, betaprodine, diphenoxylate, fentanil, sufentanil, alfentanil, remifentanil, levorphanol, dextromethorphan, phenazocine, pentazocine, cyclazocine, methadone, isomethadone and propoxyphene), hematopoietic colony stimulating factors (e.g., filgrastim, pegfilgrastim sargramostim, molgramostim and epoetin alfa), antiemetic agents (e.g., metoclopromide, domperidone, prochlorperazine, promethazine, chlorpromazine, trimethobenzamide, ondansetron, granisetron, hydroxyzine, acethylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinols, thiethylperazine, thioproperazine and tropisetron), dapsone, psoralens (e.g., methoxalen and trioxsalen), antihistamines, anti-malarial agents (e.g., hydroxychloroquine), anti-viral agents, and antibiotics (e.g., dactinomycin (formerly actinomycin), bleomycin, erythomycin, penicillin, mithramycin, and anthramycin (AMC)).

### 5.3.1 Immunodulatory Agents

Any immunomodulatory agent well-known to one of skill in the art may be used in the methods and compositions of the invention. Immunomodulatory agents can affect one or more or all aspects of the immune response in a subject. Aspects of the immune response include, but are not limited to, the inflammatory response, the complement cascade, leukocyte and lymphocyte differentiation, proliferation, and/or effector function, monocyte and/or basophil counts, and the cellular communication among cells of the immune system. In certain embodiments of the invention, an immunomodulatory agent modulates one aspect of the immune response. In other embodiments, an immunomodulatory agent modulates more than one aspect of the immune response. In a preferred embodiment of the invention, the administration of an immunomodulatory agent to a subject inhibits or reduces one or more aspects of the subject's immune response capabilities. In a specific embodiment of the invention, the immunomodulatory agent inhibits or suppresses the immune response in a subject. In accordance with the invention, an immunomodulatory agent is not a compound of the invention. In certain embodiments, an immunomodulatory agent is not an anti-inflammatory agent. In other embodiments, an immunomodulatory agent is not an anti-angiogenic agent. In yet other embodiments, an immunomodulatory agent is not a TNF-α antagonist.

In certain embodiments, an immunomodulatory agent is a chemotherapeutic agent. In other embodiments, an immunomodulatory agent is not a chemotherapeutic agent.

Examples of immunomodulatory agents include, but are not limited to, proteinaceous agents such as cytokines, peptide mimetics, and antibodies (e.g., human, humanized, chimeric, monoclonal, polyclonal, Fvs, ScFvs, Fab or F(ab)2 fragments or epitope binding fragments), nucleic acid molecules (e.g., antisense nucleic acid molecules, triple helices and nucleic acid molecules encoding immunomodulatory gene products), small molecules, organic compounds, and inorganic compounds. In particular, immunomodulatory agents include, but are not limited to, methothrexate, leflunomide, cyclophosphamide, cytoxan, Immuran, cyclosporine A, minocycline, azathioprine, antibiotics (e.g., FK506 (tacrolimus)), methylprednisolone (MP), corticosteroids, steriods, mycophenolate mofetil, rapamycin (sirolimus), mizoribine, deoxyspergualin, brequinar, malononitriloamindes (e.g., leflunamide), T cell receptor modulators, and cytokine receptor modulators.

As used herein, the term “T cell receptor modulator” refers to an agent which modulates the phosphorylation of a T cell receptor, the activation of a signal transduction pathway associated with a T cell receptor, and/or the expression of a particular protein such as a cytokine. Such an agent may directly or indirectly modulate the phosphorylation of a T cell receptor, the activation of a signal transduction pathway associated with a T cell receptor, and/or the expression of a particular protein such as a cytokine. Thus, examples of T cell receptor modulators include, but are not limited to, peptides, polypeptides, proteins, fusion proteins and antibodies which immunospecifically bind to a T cell receptor or a fragment thereof. Further, examples of T cell receptor modulators include, but are not limited to, proteins, peptides, polypeptides (e.g., soluble T cell receptors), fusion proteins and antibodies that immunospecifically binds to a ligand for a T cell receptor or a fragment thereof. Examples of T cell receptor modulators include, but are not limited to, anti-T cell receptor antibodies (e.g., anti-CD4 antibodies (e.g., cM-T412 (Boeringer), IDEC-CE9.1® (IDEC and SKB), mAB 4162W94, Orthoclone and OKTcdr4a (Janssen-Cilag)), anti-CD3 antibodies (e.g., Nuvion (Product Design Labs), OKT3 (Johnson & Johnson), or Rituxan (IDEC)), anti-CD5 antibodies (e.g., an anti-CD5 ricin-linked immunoconjugate), anti-CD7 antibodies (e.g., CHH-380 (Novartis)), anti-CD8 antibodies, anti-CD40 ligand monoclonal antibodies (e.g., IDEC-131 (IDEC)), anti-CD52 antibodies (e.g., CAMPATH 1H (Ilex)), anti-CD2 antibodies, anti-CD11a antibodies (e.g., Xanelim (Genentech)), and anti-B7 antibodies (e.g., IDEC-1 14) (IDEC))), CTLA4-immunoglobulin, and LFA-3TIP (Biogen, International Publication No. WO 93/08656 and U.S. Pat. No. 6,162,432).

As used herein, the term “cytokine receptor modulator” refers to an agent which modulates the phosphorylation of a cytokine receptor, the activation of a signal transduction pathway associated with a cytokine receptor, and/or the expression of a particular protein such as a cytokine. Such an agent may directly or indirectly modulate the phosphorylation of a cytokine receptor, the activation of a signal transduction pathway associated with a cytokine receptor, and/or the expression of a particular protein such as a cytokine. Thus, examples of cytokine receptor modulators include, but are not limited to, cytokines, fragments of cytokines, fusion proteins and antibodies that immunospecifically binds to a cytokine receptor or a fragment thereof. Further, examples of cytokine receptor modulators include, but are not limited to, peptides, polypeptides (e.g., soluble cytokine receptors), fusion proteins and antibodies that immunospecifically binds to a cytokine or a fragment thereof. Examples of cytokine receptor modulators include, but are not limited to, soluble cytokine receptors (e.g., the extracellular domain of a TNF-α receptor or a fragment thereof, the extracellular domain of an IL-1β receptor or a fragment thereof, and the extracellular domain of an IL-6 receptor or a fragment thereof), cytokines or fragments thereof (e.g., interleukin (IL)-2, IL-3, IL-4, IL-5, IL-6, IL-7, IL-8, IL-9, IL-10, IL-11, IL-12, IL-15, IL-23 TNF-α, TNF-β, interferon (IFN)-α, IFN-β, IFN-γ, and GM-CSF), anti-cytokine receptor antibodies (e.g., anti-IFN receptor antibodies, anti-IL-2 receptor antibodies (e.g., Zenapax (Protein Design Labs)), anti-IL-4 receptor antibodies, anti-IL-6 receptor antibodies, anti-IL-10 receptor antibodies, anti-IL-12 receptor antibodies, anti-IL-15 receptor antibodies and anti-IL-23 receptor antibodies), anti-cytokine antibodies (e.g., anti-IFN α antibodies, anti-IFN-β antibodies, anti-IFN-γ antibodies, anti-TNF-α antibodies, anti-IL-1β antibodies, anti-IL-2 antibodies, anti-IL-4 antibodies, anti-IL-6 antibodies, anti-IL-8 antibodies (e.g., ABX-IL-8 (Abgenix)), anti-IL-9 antibodies, anti-IL-10 antibodies, anti-IL-12 antibodies and anti-IL-23 antibodies). In a specific embodiment, a cytokine receptor modulator is IL-4, IL-10, or a fragment thereof. In another embodiment, a cytokine receptor modulator is an anti-IL-1β antibody, anti-IL-6 antibody, anti-IL-12 receptor antibody, or anti-TNF-α antibody. In another embodiment, a cytokine receptor modulator is the extracellular domain of a TNF-α receptor or a fragment thereof. In certain embodiments, a cytokine receptor modulator is not a TNF-α antagonist.

An immunomodulatory agent may be selected to interfere with the interactions between the T helper subsets (TH1 or TH2) and B cells to inhibit neutralizing antibody formation. An immunomodulatory agent may also be selected to inhibit the interaction between TH1 cells and cytoxic T cells (CTLs) to reduce the occurrence of CTL-mediated killing. Further, an immunomodulatory agent may be selected to alter (e.g., inhibit or suppress) the proliferation, differentiation, activity and/or function of the CD4+and/or CD8⁺ T cells. For example, antibodies specific for T cells can be used as immunomodulatory agents to deplete, or alter the proliferation, differentiation, activity and/or function of CD4⁺ and/or CD8⁺ T cells.

In one embodiment of the invention, an immunomodulatory agent that reduces or depletes T cells, preferably memory T cells, is administered to a subject with a proliferative disorder or an inflammatory disorder in accordance with the methods of the invention. See, e.g., U.S. Pat. No. 4,658,019. In another embodiment of the invention, an immunomodulatory agent that inactivates CD8⁺ T cells is administered to a subject with a proliferative disorder or an inflammatory disorder in accordance with the methods of the invention. In a specific embodiment, anti-CD8 antibodies are used to reduce or deplete CD8⁺ T cells.

Antibodies that interfere with or block the interactions necessary for the activation of B cells by TH (T helper) cells, and thus block the production of neutralizing antibodies, are useful as immunomodulatory agents in accordance the methods of the invention. For example, B cell activation by T cells requires certain interactions to occur (Durie et al, Immunol. Today, 15(9):406-410 (1994)), such as the binding of CD40 ligand on the T helper cell to the CD40 antigen on the B cell, and the binding of the CD28 and/or CTLA4 ligands on the T cell to the B7 antigen on the B cell. Without both interactions, the B cell cannot be activated to induce production of the neutralizing antibody.

The CD40 ligand (CD40L)-CD40 interaction is a desirable point to block the immune response because of its broad activity in both T helper cell activation and function as well as the absence of redundancy in its signaling pathway. Thus, in a specific embodiment of the invention, the interaction of CD40L with CD40 is transiently blocked at the time of administration of one or more of the immunomodulatory agents. This can be accomplished by treating with an agent which blocks the CD40 ligand on the TH cell and interferes with the normal binding of CD40 ligand on the T helper cell with the CD40 antigen on the B cell. An antibody to CD40 ligand (anti-CD40L) (available from Bristol-Myers Squibb Co; see, e.g., European patent application 555,880, published Aug. 18, 1993) or a soluble CD40 molecule can be selected and used as an immunomodulatory agent in accordance with the methods of the invention.

In another embodiment, an immunomodulatory agent which reduces or inhibits one or more biological activities (e.g., the differentiation, proliferation, and/or effector functions) of TH0, TH1, and/or TH2 subsets of CD4⁺ T helper cells is administered to a subject with an inflammatory disorder or a proliferative disorder or an infection in accordance with the methods of the invention. One example of such an immunomodulatory agent is IL-4. IL-4 enhances antigen-specific activity of TH2 cells at the expense of the TH1 cell function (see, e.g., Yokota et al., 1986 Proc. Natl. Acad. Sci., USA, 83:5894-5898; and U.S. Pat. No. 5,017,691). Other examples of immunomodulatory agents that affect the biological activity (e.g., proliferation, differentiation, and/or effector functions) of T-helper cells (in particular, TH1 and/or TH2 cells) include, but are not limited to, IL-2, IL-4, IL-5, IL-6, IL-9, IL-10, IL-12, IL-13, IL-15, and interferon (IFN)-γ.

In a preferred embodiment, proteins, polypeptides or peptides (including antibodies) that are utilized as immunomodulatory agents are derived from the same species as the recipient of the proteins, polypeptides or peptides so as to reduce the likelihood of an immune response to those proteins, polypeptides or peptides. In another preferred embodiment, when the subject is a human, the proteins, polypeptides, or peptides that are utilized as immunomodulatory agents are human or humanized.

In accordance with the invention, one or more immunomodulatory agents are administered to a subject with a disorder disorder (e.g., a disorder characterized by or associated with aberrant angiogensis, a proliferative disorder, an inflammatory disorder or a disorder prevented, managed, treated or ameliorated by inhibiting NF-κB activation and phosphorylation of p44/42 MAPK, or by reducing or inhibiting production of NO, IL-1β, TNF-α and expression of iNOS and Cox-2 gene expression) prior to, subsequent to, or concomitantly with a compound of the invention. Preferably, one or more immunomodulatory agents are administered to a subject with a proliferative disorder or an inflammatory disorder in combination with a compound of the invention to reduce or inhibit one or more aspects of the immune response. Any technique well-known to one skilled in the art can be used to measure one or more aspects of the immune response in a particular subject, and thereby determine when to administer an immunomodulatory agent to said subject. In a preferred embodiment, a mean absolute lymphocyte count of approximately 500 cells/mm³, preferably 600 cells/mm³, 650 cells/mm³, 700 cells/mm³, 750 cells/mm³, 800 cells/mm³, 900 cells/mm³, 1000 cells/mm³, 1100 cells/mm³, or 1200 cells/mm³ is maintained in a subject. In another preferred embodiment, a subject with a proliferative disorder or an inflammatory disorder is not administered an immunomodulatory agent if their absolute lymphocyte count is 500 cells/mm³ or less, 550 cells/mm³ or less, 600 cells/mm³ or less, 650 cells/mm³ or less, 700 cells/mm³ or less, 750 cells/mm³ or less, or 800 cells/mm³ or less.

In a preferred embodiment, one or more immunomodulatory agents are administered to a subject with a disorder (e.g., a disorder characterized by or associated with aberrant angiogensis, a proliferative disorder, an inflammatory disorder or a disorder prevented, managed, treated or ameliorated by inhibiting NF-κB activation and phosphorylation of p44/42 MAPK, or by reducing or inhibiting production of NO, IL-1β, TNF-α and expression of iNOS and Cox-2 gene expression) in combination with a compound of the invention so as to transiently reduce or inhibit one or more aspects of the immune response. Such a transient inhibition or reduction of one or more aspects of the immune system can last for hours, days, weeks, or months. Preferably, the transient inhibition or reduction in one or more aspects of the immune response last for a few hours (e.g., 2 hours, 4 hours, 6 hours, 8 hours, 12 hours, 14 hours, 16 hours, 18 hours, 24 hours, 36 hours, or 48 hours), a few days (e.g., 3 days, 4 days, 5 days, 6 days, 7 days, or 14 days), or a few weeks (e.g., 3 weeks, 4 weeks, 5 weeks or 6 weeks). The transient reduction or inhibition of one or more aspects of the immune response enhances the prophylactic and/or therapeutic capabilities of a compound of the invention.

Nucleic acid molecules encoding proteins, polypeptides, or peptides with immunomodulatory activity or proteins, polypeptides, or peptides with immunomodulatory activity can be administered to a subject with a disorder (e.g., a disorder characterized by or associated with aberrant angiogensis, a proliferative disorder, an inflammatory disorder or a disorder prevented, managed, treated or ameliorated by inhibiting NF-κB activation and phosphorylation of p44/42 MAPK, or by reducing or inhibiting production of NO, IL-1β, TNF-α and expression of iNOS and Cox-2 gene expression) in accordance with the methods of the invention. Further, nucleic acid molecules encoding derivatives, analogs, or fragments of proteins, polypeptides, or peptides with immunomodulatory activity, or derivatives, analogs, or fragments of proteins, polypeptides, or peptides with immunomodulatory activity can be administered to a subject with a disorder (e.g., a disorder characterized by or associated with aberrant angiogensis, a proliferative disorder, an inflammatory disorder or a disorder prevented, managed, treated or ameliorated by inhibiting NF-κB activation and phosphorylation of p44/42 MAPK, or by reducing or inhibiting production of NO, IL-1β, TNF-α and expression of iNOS and Cox-2 gene expression) in accordance with the methods of the invention. Preferably, such derivatives, analogs, and fragments retain the immunomodulatory activity of the full-length, wild-type protein, polypeptide, or peptide.

Proteins, polypeptides, or peptides that can be used as anti-angiogenic agents can be produced by any technique well-known in the art or described herein. Proteins, polypeptides or peptides with immunomodulatory activity can be engineered so as to increase the in vivo half-life of such proteins, polypeptides, or peptides utilizing techniques well-known in the art or described herein. Preferably, agents that are commercially available and known to function as immunomoulatory agents are used in the compositions and methods of the invention. The immunomodulatory activity of an agent can be determined in vitro and/or in vivo by any technique well-known to one skilled in the art, including, e.g., by CTL assays (⁵¹Cr release assays), proliferation assays (³H-thymidine incorporation or trypan blue cell counts), northern blot assays, and immunoassays (e.g. ELISAs and western blot expression) for the expression of particular gene products (e.g., RNA or proteins) such as co-stimulatory molecules and cytokines.

Immunomodulatory agents and their dosages, routes of administration and recommended usage are known in the art and have been described in such literature as the Physician's Desk Reference (58^{th} ed., 2004).

### 5.3.2 Anti-Angiogenic Agents

Any anti-angiogenic agent well-known to one of skill in the art can be used in the compositions and methods of the invention. Non-limiting examples anti-angiogenic agents include proteins, polypeptides, peptides, fusion proteins, antibodies (e.g., human, humanized, chimeric, monoclonal, polyclonal, Fvs, ScFvs, Fab fragments, F(ab)₂ fragments, and antigen-binding fragments thereof) such as antibodies that immunospecifically bind to TNF-α, nucleic acid molecules (e.g., antisense molecules or triple helices), organic molecules, inorganic molecules, and small molecules that reduce or inhibit angiogenesis. In particular, examples of anti-angiogenic agents, include, but are not limited to, endostatin, angiostatin, apomigren, anti-angiogenic antithrombin III, the 29 kDa N-terminal and a 40 kDa C-terminal proteolytic fragments of fibronectin, a uPA receptor antagonist, the 16 kDa proteolytic fragment of prolactin, the 7.8 kDa proteolytic fragment of platelet factor-4, the anti-angiogenic 24 amino acid fragment of platelet factor-4, the anti-angiogenic factor designated 13.40, the anti-angiogenic 22 amino acid peptide fragment of thrombospondin I, the anti-angiogenic 20 amino acid peptide fragment of SPARC, RGD and NGR containing peptides, the small anti-angiogenic peptides of laminin, fibronectin, procollagen and EGF, anti-integrin αᵥβ₃ antibodies, acid fibroblast growth factor (aFGF) antagonists, basic fibroblast growth factor (bFGF) antagonists, vascular endothelial growth factor (VEGF) antagonists (e.g., anti-VEGF antibodies such as Avastin®), and VEGF receptor (VEGFR) antagonists (e.g., anti-VEGFR antibodies).

Examples of integrin αᵥβ₃ antagonists include, but are not limited to, proteinaceous agents such as non-catalytic metalloproteinase fragments, RGD peptides, peptide mimetics, fusion proteins, disintegrins or derivatives or analogs thereof, and antibodies that immunospecifically bind to integrin αᵥβ₃, nucleic acid molecules, organic molecules, and inorganic molecules. Non-limiting examples of antibodies that immunospecifically bind to integrin αᵥβ₃ include 11D2 (Searle). Non-limiting examples of small molecule peptidometric integrin αᵥβ₃ antagonists include S836 (Searle) and S448 (Searle). Examples of disintegrins include, but are not limited to, Accutin. The invention also encompasses the use of any of the integrin αᵥβ₃ antagonists disclosed in the following U.S. Patents and International publications in the compositions and methods of the invention: U.S. Pat. Nos. 5,652,109; 5,652,110; 5,578,704; 5,149,780; 5,196,511; 5,204,445; 5,262,520; 5,306,620; 5,478,725; 5,498,694; 5,523,209; 5,578,704; 5,589,570; 5,652,109; 5,652,110; 5,693,612; 5,705,481; 5,753,230; 5,767,071; 5,770,565; 5,780,426; 5,817,457; 5,830,678; 5,849,692; 5,955,572; 5,985,278; 6,048,861; 6,090,944; 6,096,707; 6,130,231; 6,153,628; 6,160,099; and 6,171,58; and International Publication Nos. WO 95/22543; WO 98/33919; WO 00/78815; WO 00/31248; WO 98/46264; WO 98/40488; and WO 02/070007, each of which is incorporated herein by reference in its entirety.

In a specific embodiment of the invention, an anti-angiogenic agent is endostatin. Naturally occurring endostatin consists of the C-terminal ˜180 amino acids of collagen XVIII (cDNAs encoding two splice forms of collagen XVIII have GenBank Accession Nos. AF18081 and AF18082). In another embodiment of the invention, an anti-angiogenic agent is a plasminogen fragment (the coding sequence for plasminogen can be found in GenBank Accession Nos. NM_{—}000301 and A33096). Angiostatin peptides naturally include the four kringle domains of plasminogen, kringle 1 through kringle 4. It has been demonstrated that recombinant kringle 1, 2 and 3 possess the anti-angiogenic properties of the native peptide, whereas kringle 4 has no such activity (Cao et al., 1996, J. Biol. Chem. 271:29461-29467). Accordingly, the angiostatin peptides comprises at least one and preferably more than one kringle domain selected from the group consisting of kringle 1, kringle 2 and kringle 3. In a specific embodiment, the anti-angiogenic peptide is the 40 kDa isoform of the human angiostatin molecule, the 42 kDa isoform of the human angiostatin molecule, the 45 kDa isoform of the human angiostatin molecule, or a combination thereof. In another embodiment, an anti-angiogenic agent is the kringle 5 domain of plasminogen, which is a more potent inhibitor of angiogenesis than angiostatin (angiostatin comprises kringle domains 1-4). In another embodiment of the invention, an anti-angiogenic agent is antithrombin III. Antithrombin III, which is referred to hereinafter as antithrombin, comprises a heparin binding domain that tethers the protein to the vasculature walls, and an active site loop which interacts with thrombin. When antithrombin is tethered to heparin, the protein elicits a conformational change that allows the active loop to interact with thrombin, resulting in the proteolytic cleavage of said loop by thrombin. The proteolytic cleavage event results in another change of conformation of antithrombin, which (i) alters the interaction interface between thrombin and antithrombin and (ii) releases the complex from heparin (Carrell, 1999, Science 285:1861-1862, and references therein). O'Reilly et al. (1999, Science 285:1926-1928) have discovered that the cleaved antithrombin has potent anti-angiogenic activity. Accordingly, in one embodiment, an anti-angiogenic agent is the anti-angiogenic form of antithrombin. In another embodiment of the invention, an anti-angiogenic agent is the 40 kDa and/or 29 kDa proteolytic fragment of fibronectin.

In another embodiment of the invention, an anti-angiogenic agent is a urokinase plasminogen activator (uPA) receptor antagonist. In one mode of the embodiment, the antagonist is a dominant negative mutant of uPA (see, e.g., Crowley et al., 1993, Proc. Natl. Acad. Sci. USA 90:5021-5025). In another mode of the embodiment, the antagonist is a peptide antagonist or a fusion protein thereof (Goodson et al., 1994, Proc. Natl. Acad. Sci. USA 91:7129-7133). In yet another mode of the embodiment, the antagonist is a dominant negative soluble uPA receptor (Min et al., 1996, Cancer Res. 56:2428-2433). In another embodiment of the invention, an anti-angiogenic agent is the 16 kDa N-terminal fragment of prolactin, comprising approximately 120 amino acids, or a biologically active fragment thereof (the coding sequence for prolactin can be found in GenBank Accession No. NM_{—}000948). In another embodiment of the invention, an anti-angiogenic agent is the 7.8 kDa platelet factor-4 fragment. In another embodiment of the invention, an anti-angiogenic agent is a small peptide corresponding to the anti-angiogenic 13 amino acid fragment of platelet factor-4, the anti-angiogenic factor designated 13.40, the anti-angiogenic 22 amino acid peptide fragment of thrombospondin I, the anti-angiogenic 20 amino acid peptide fragment of SPARC, the small anti-angiogenic peptides of laminin, fibronectin, procollagen, or EGF, or small peptide antagonists of integrin αᵥβ₃ or the VEGF receptor. In another embodiment, the small peptide comprises an RGD or NGR motif. In certain embodiments, an anti-angiogenic agent is a TNF-α antagonist. In other embodiments, an anti-angiogenic agent is not a TNF-α antagonist.

Nucleic acid molecules encoding proteins, polypeptides, or peptides with anti-angiogenic activity, or proteins, polypeptides or peptides with anti-angiogenic activity can be administered to a subject with a disorder (e.g., a disorder characterized by or associated with aberrant angiogensis, a proliferative disorder, an inflammatory disorder or a disorder prevented, managed, treated or ameliorated by inhibiting NF-κB activation and phosphorylation of p44/42 MAPK, or by reducing or inhibiting production of NO, IL-1β, TNF-α and expression of iNOS and Cox-2 gene expression) in accordance with the methods of the invention. Further, nucleic acid molecules encoding derivatives, analogs, fragments, or variants of proteins, polypeptides, or peptides with anti-angiogenic activity, or derivatives, analogs, fragments, or variants of proteins, polypeptides, or peptides with anti-angiogenic activity can be administered to a subject with a disorder (e.g., a disorder characterized by or associated with aberrant angiogensis, a proliferative disorder, an inflammatory disorder or a disorder prevented, managed, treated or ameliorated by inhibiting NF-κB activation and phosphorylation of p44/42 MAPK, or by reducing or inhibiting production of NO, IL-1β, TNF-α and expression of iNOS and Cox-2 gene expression) in accordance with the methods of the invention. Preferably, such derivatives, analogs, variants, and fragments retain the anti-angiogenic activity of the full-length, wild-type protein, polypeptide, or peptide.

Proteins, polypeptides, or peptides that can be used as anti-angiogenic agents can be produced by any technique well-known in the art or described herein. Proteins, polypeptides or peptides with anti-angiogenic activity can be engineered so as to increase the in vivo half-life of such proteins, polypeptides, or peptides utilizing techniques well-known in the art or described herein. Preferably, anti-angiogenic agents that are commercially available are used in the compositions and methods of the invention. The anti-angiogenic activity of an agent can be determined in vitro and/or in vivo by any technique well-known to one skilled in the art or described herein.

Anti-angiogenic agents and their dosages, routes of administration and recommended usage are known in the art and have been described in such literature as the Physician's Desk Reference (58^{th} ed., 2004).

### 5.3.3 TNF-α Antagonists

Any TNF-α antagonist well-known to one of skill in the art can be used in the compositions and methods of the invention. Non-limiting examples of TNF-α antagonists include proteins, polypeptides, peptides, fusion proteins, antibodies (e.g., human, humanized, chimeric, monoclonal, polyclonal, Fvs, ScFvs, Fab fragments, F(ab)₂ fragments, and antigen-binding fragments thereof) such as antibodies that immunospecifically bind to TNF-α, nucleic acid molecules (e.g., antisense molecules or triple helices), organic molecules, inorganic molecules, and small molecules that block, reduce, inhibit or neutralize a function, an activity and/or the expression of TNF-α. In various embodiments, a TNF-α antagonist reduces the function, activity and/or expression of TNF-α by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% relative to a control such as phosphate buffered saline (PBS).

Examples of antibodies that immunospecifically bind to TNF-α include, but are not limited to, infliximab (REMICADE®; Centacor), D2E7 (Abbott Laboratories/Knoll Pharmaceuticals Co., Mt. Olive, N.J.), CDP571 which is also known as HUMICADE™ and CDP-870 (both of Celitech/Pharmacia, Slough, U.K.), and TN3-19.12 (Williams et al., 1994, Proc. Natl. Acad. Sci. USA 91: 2762-2766; Thorbecke et al., 1992, Proc. Natl. Acad. Sci. USA 89:7375-7379). The present invention also encompasses the use of the antibodies that immunospecifically bind to TNF-α disclosed in the following U.S. Patents in the compositions and methods of the invention: U.S. Pat. Nos. 5,136,021; 5,147,638; 5,223,395; 5,231,024; 5,334,380; 5,360,716; 5,426,181; 5,436,154; 5,610,279; 5,644,034; 5,656,272; 5,658,746; 5,698,195; 5,736,138; 5,741,488; 5,808,029; 5,919,452; 5,958,412; 5,959,087; 5,968,741; 5,994,510; 6,036,978; 6,114,517; and 6,171,787; each of which are herein incorporated by reference in their entirety. Examples of soluble TNF-α receptors include, but are not limited to, sTNF-R1 (Amgen), etanercept (ENBREL™; rmnunex) and its rat homolog RENBREL™, soluble inhibitors of TNF-α derived from TNFrI, TNFrII (Kohno et al., 1990, Proc. Natl. Acad. Sci. USA 87:8331-8335), and TNF-α Inh (Seckinger et al., 1990, Proc. Natl. Acad. Sci. USA 87:5188-5192).

In one embodiment, a TNF-α antagonist used in the compositions and methods of the invention is a soluble TNF-α receptor. In a specific embodiment, a TNF-α antagonist used in the compositions and methods of the invention is etanercept (ENBREL™; Immunex) or a fragment, derivative or analog thereof. In another embodiment, a TNF-α antagonist used in the compositions and methods of the invention is an antibody that immunospecifically binds to TNF-α. In a specific embodiment, a TNF-α antagonist used in the compositions and methods of the invention is infliximab (REMICADE®; Centacor) a derivative, analog or antigen-binding fragment thereof.

Other TNF-α antagonists encompassed by the invention include, but are not limited to, IL-10, which is known to block TNF-α production via interferon γ-activated macrophages (Oswald et al. 1992, Proc. Natl. Acad. Sci. USA 89:8676-8680), TNFR-IgG (Ashkenazi et al., 1991, Proc. Natl. Acad. Sci. USA 88:10535-10539), the murine product TBP-1 (Serono/Yeda), the vaccine CytoTAb (Protherics), antisense molecule 104838 (ISIS), the peptide RDP-58 (SangStat), thalidomide (Celgene), CDC-801 (Celgene), DPC-333 (Dupont), VX-745 (Vertex), AGIX-4207 (AtheroGenics), ITF-2357 (Italfarmaco), NPI-13021-31 (Nereus), SCIO-469 (Scios), TACE targeter (Immunix/AHP), CLX-120500 (Calyx), Thiazolopyrim (Dynavax), auranofin (Ridaura) (SmithKline Beecham Pharmaceuticals), quinacrine (mepacrine dichlorohydrate), tenidap (Enablex), Melanin (Large Scale Biological), and anti-p38 MAPK agents by Uriach.

Nucleic acid molecules encoding proteins, polypeptides, or peptides with TNF-α antagonist activity, or proteins, polypeptides, or peptides with TNF-α antagonist activity can be administered to a subject with a disorder (e.g., a disorder characterized by or associated with aberrant angiogensis, a proliferative disorder, an inflammatory disorder or a disorder prevented, managed, treated or ameliorated by inhibiting NF-κB activation and phosphorylation of p44/42 MAPK, or by reducing or inhibiting production of NO, IL-1β, TNF-α and expression of iNOS and Cox-2 gene expression) in accordance with the methods of the invention. Further, nucleic acid molecules encoding derivatives, analogs, fragments or variants of proteins, polypeptides, or peptides with TNF-α antagonist activity, or derivatives, analogs, fragments or variants of proteins, polypeptides, or peptides with TNF-α antagonist activity can be administered to a subject with a disorder (e.g., a disorder characterized by or associated with aberrant angiogensis, a proliferative disorder, an inflammatory disorder or a disorder prevented, managed, treated or ameliorated by inhibiting NF-κB activation and phosphorylation of p44/42 MAPK, or by reducing or inhibiting production of NO, IL-1β, TNF-α and expression of iNOS and Cox-2 gene expression) in accordance with the methods of the invention. Preferably, such derivatives, analogs, variants and fragments retain the TNF-α antagonist activity of the full-length, wild-type protein, polypeptide, or peptide.

Proteins, polypeptides, or peptides that can be used as TNF-α antagonists can be produced by any technique well-known in the art or described herein. Proteins, polypeptides or peptides with TNF-α antagonist activity can be engineered so as to increase the in vivo half-life of such proteins, polypeptides, or peptides utilizing techniques well-known in the art or described herein. Preferably, agents that are commercially available and known to function as TNF-α antagonists are used in the compositions and methods of the invention. The TNF-α antagonist activity of an agent can be determined in vitro and/or in vivo by any technique well-known to one skilled in the art.

TNF-α antagonists and their dosages, routes of administration and recommended usage are known in the art and have been described in such literature as the Physician's Desk Reference (58th ed., 2004).

### 5.3.4 Anti-Inflammatory Agents

Anti-inflammatory agents have exhibited success in treatment of proliferative disorders or inflammatory disorders and are now a common and a standard treatment for such disorders as well as others. Any anti-inflammatory therapy (e.g., an anti-inflammatory agent) well-known to one of skill in the art can be used in the compositions and methods of the invention. Non-limiting examples of anti-inflammatory agents include non-steroidal anti-inflammatory drugs (NSAIDs), steroidal anti-inflammatory drugs, beta-agonists, anticholingeric agents, antihistamines (e.g., ethanolamines, ethylenediamines, piperazines, and phenothiazine), and methyl xanthines. Examples of NSAIDs include, but are not limited to, aspirin, ibuprofen, salicylates, acetominophen, celecoxib (CELEBREX™), diclofenac (VOLTAREN™), etodolac (LODINE™), fenoprofen (NALFON™), indomethacin (INDOCIN™), ketoralac (TORADOL™), oxaprozin (DAYPRO™), nabumentone (RELAFEN™), sulindac (CLINORIL™), tolmentin (TOLECTIN™), rofecoxib (VIOXX™), naproxen (ALEVE™, NAPROSYN™), ketoprofen (ACTRON™) and nabumetone (RELAFEN™). Such NSAIDs function by inhibiting a cyclooxgenase enzyme (e.g., COX-1 and/or COX-2). Examples of steroidal anti-inflammatory drugs include, but are not limited to, glucocorticoids, dexamethasone (DECADRON™), cortisone, hydrocortisone, prednisone (DELTASONE™), prednisolone, triamcinolone, azulfidine, and eicosanoids such as prostaglandins, thromboxanes, and leukotrienes.

Anti-inflammatory agents and their dosages, routes of administration and recommended usage are known in the art and have been described in such literature as the Physician's Desk Reference (58^{th} ed., 2004).

### 5.3.5 Anti-Cancer Agents

Any therapy (e.g., any prophylactic or therapeutic agent) which is known to be useful, has been used, or is currently being used for the prevention, treatment, management, or amelioration of one or more symptoms associated with a proliferative disorder, such as cancer can be used in compositions and method of the invention. Therapeutic or prophylactic agents include, but are not limited to, peptides, polypeptides, fusion proteins, nucleic acid molecules, small molecules, mimetic agents, synthetic drugs, inorganic molecules, and organic molecules. Non-limiting examples of cancer therapies include chemotherapies, radiation therapies, hormonal therapies, and/or biological therapies/immunotherapies.

In certain embodiments, the anti-cancer agent is an immunomodulatory agent such as a chemotherapeutic agent. In other embodiments, the anti-cancer agent is not an immunomodulatory agent. In specific embodiments, the anti-cancer agent is an anti-angiogenic agent. In other embodiments, the anti-cancer agent is not an anti-angiogenic agent.

Examples of anti-cancer agents include, but arenot limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bisphosphonates (e.g., pamidronate (Aredria), sodium clondronate (Bonefos), zoledronic acid (Zometa), alendronate (Fosamax), etidronate, ibandomate, cimadronate, risedromate, and tiludromate); bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin-2 (including recombinant interleukin 2, or rIL2), interferon alpha-2a; interferon alpha-2b; interferon alpha-nl; interferon alpha-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; anti-CD2 antibodies; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; Avastin®; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidernin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; HMG CoA reductase inhibitors (e.g., atorvastatin, cerivastatin, fluvastatin, lescol, lupitor, lovastatin, rosuvastatin, and simvastatin); hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-I receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; LFA-3T1P (Biogen, Cambridge, Mass.; U.S. Pat. No. 6,162,432); liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; 5-fluorouracil; leucovorin; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremnifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; thalidomide; velaresol; veramine; verdins; verteporfin; vinorelbine; vinxaltine; vorozole; zanoterone; zeniplatin; zilascorb; and zinostatin stimalamer.

In more particular embodiments, the present invention also comprises the administration of a compound of the invention in combination with the administration of one or more therapies such as, but not limited to anti-cancer agents such as those disclosed in Table 1, preferably for the treatment of breast, ovary, melanoma, prostate, colon and lung cancers.

TABLE 1

| Therapeutic | | | |
|---|---|---|---|
| Agent | Dose/Administration/Formulation | | |
| Doxorubicin | Intravenous | 60-75 mg/m² | 21 day |
| hydrochloride | | on Day 1 | intervals |
| (Adriamycin | | | |
| RDF ® and | | | |
| Adriamycin | | | |
| PFS ®) | | | |
| Epirubicin | Intravenous | 100-120 mg/m² | 3-4 week |
| hydrochloride | | on Day 1 of each | cycles |
| (Ellence ™) | | cycle or divided | |
| | | equally and given | |
| | | on Days 1-8 of | |
| | | the cycle | |
| Fluorousacil | Intravenous | How supplied: | |
| | | 5 mL and 10 mL | |
| | | vials (containing | |
| | | 250 and 500 mg | |
| | | flourouracil | |
| | | respectively) | |
| Docetaxel | Intravenous | 60-100 mg/m² | Once every |
| (Taxotere ®) | | over 1 hour | 3 weeks |
| Paclitaxel | Intravenous | 175 mg/m² over 3 | Every 3 weeks |
| (Taxol ®) | | hours | for 4 courses |
| | | | (administered |
| | | | sequentially |
| | | | to doxorubicin- |
| | | | containing |
| | | | combination |
| | | | chemotherapy) |
| tamoxifen | Oral | 20-40 mg | Daily |
| citrate | (tablet) | Dosages greater | |
| (Nolvadex ®) | | than 20 mg should | |
| | | be given in | |
| | | divided doses | |
| | | (morning and | |
| | | evening) | |
| leucovorin | Intravenous | How supplied: | Dosage is un- |
| calcium for | or intra- | 350 mg vial | clear from |
| injection | muscular | | text. PDR 3610 |
| | injection | | |
| luprolide | Single | 1 mg (0.2 mL or | Once a day |
| acetate | subcutaneous | 20 unit mark) | |
| (Lupron ®) | injection | | |
| Flutamide | Oral | 250 mg | 3 times a day |
| (Eulexin ®) | (capsule) | (capsules | at 8 hour |
| | | contain 125 mg | intervals |
| | | flutamide each) | (total daily |
| | | | dosage 750 mg) |
| Nilutamide | Oral | 300 mg or 150 mg | 300 mg once a |
| (Nilandron ®) | (tablet) | (tablets contain | day for 30 |
| | | 50 or 150 mg | days followed |
| | | nilutamide each) | by 150 mg once |
| | | | a day |
| Bicalutamide | Oral | 50 mg | Once a day |
| (Casodex ®) | (tablet) | (tablets contain | |
| | | 50 mg | |
| | | bicalutamide | |
| | | each) | |
| Progesterone | Injection | USP in sesame oil | |
| | | 50 mg/mL | |
| Ketoconazole | Cream | 2% cream applied | |
| (Nizoral ®) | | once or twice | |
| | | daily depending | |
| | | on symptoms | |
| prednisone | Oral | Initial dosage | |
| | (tablet) | may vary from | |
| | | 5 mg to 60 mg | |
| | | per day depending | |
| | | on the specific | |
| | | disease entity | |
| | | being treated. | |
| Estramustine | Oral | 14 mg/kg of body | Daily given in |
| phosphate | (capsule) | weight (i.e. one | 3 or 4 divided |
| sodium | | 140 mg capsule | doses |
| (Emcyt ®) | | for each 10 kg | |
| | | or 22 lb of body | |
| | | weight) | |
| etoposide | Intravenous | 5 mL of 20 mg/mL | |
| or VP-16 | | solution (100 mg) | |
| Dacarbazine | Intravenous | 2-4.5 mg/kg | Once a day for |
| (DTIC- | | | 10 days. |
| Dome ®) | | | May be |
| | | | repeated at 4 |
| | | | week intervals |
| Polifeprosan | wafer placed | 8 wafers, each | |
| 20 with | in resection | containing 7.7 mg | |
| carmustine | cavity | of carmustine, | |
| implant (BCNU) | | for a total of | |
| (nitrosourea) | | 61.6 mg, if size | |
| (Gliadel ®) | | and shape of | |
| | | resection | |
| | | cavity allows | |
| Cisplatin | Injection | [n/a in PDR | |
| | | 861] | |
| | | How supplied: | |
| | | solution of | |
| | | 1 mg/mL in multi- | |
| | | dose vials of | |
| | | 50 mL and 100 mL | |
| Mitomycin | Injection | supplied in 5 mg | |
| | | and 20 mg vials | |
| | | (containing 5 mg | |
| | | and 20 mg | |
| | | mitomycin) | |
| gemcitabine | Intravenous | For NSCLC- 2 | 4 week |
| HCl | | schedules have | schedule- |
| (Gemzar ®) | | been investigated | Days 1, 8 and |
| | | and the optimum | 15 of each 28- |
| | | schedule has not | day cycle. |
| | | been determined | Cisplatin |
| | | 4 week schedule- | intravenously |
| | | administration | at 100 mg/m² |
| | | intravenously at | on day 1 after |
| | | 1000 mg/m² over | the infusion of |
| | | 30 minutes on 3 | Gemzar. 3 week |
| | | week schedule- | schedule- |
| | | Gemzar | Days 1 and 8 |
| | | administered | of each 21 day |
| | | intravenously at | cycle. Cis- |
| | | 1250 mg/m² over | platin at |
| | | 30 minutes | dosage of |
| | | | 100 mg/m² |
| | | | administered |
| | | | intravenously |
| | | | after |
| | | | administration |
| | | | of Gemzar on |
| | | | day 1. |
| Carboplatin | Intravenous | Single agent | Every 4 weeks |
| (Paraplatin ®) | | therapy: | |
| | | 360 mg/m² I.V. | |
| | | on day 1 | |
| | | (infusion lasting | |
| | | 15 minutes or | |
| | | longer) | |
| | | Other dosage | |
| | | calculations: | |
| | | Combination | |
| | | therapy with | |
| | | cyclophosphamide, | |
| | | Dose adjustment | |
| | | recommendations, | |
| | | Formula dosing, | |
| | | etc. | |
| Ifosamide | Intravenous | 1.2 g/m² daily | 5 consecutive |
| (Ifex ®) | | | days |
| | | | Repeat every 3 |
| | | | weeks or after |
| | | | recovery from |
| | | | hematologic |
| | | | toxicity |
| Topotecan | Intravenous | 1.5 mg/m² by | 5 consecutive |
| hydrochloride | | intravenous | days, starting |
| (Hycamtin ®) | | infusion over | on day 1 of 21 |
| | | 30 minutes daily | day course |

In specific embodiments, radiation therapy comprising the use of x-rays, gamma rays and other sources of radiation to destroy the cancer cells is used in combination with the antibodies of the invention. In preferred embodiments, the radiation treatment is administered as external beam radiation or teletherapy, wherein the radiation is directed from a remote source. In other preferred embodiments, the radiation treatment is administered as internal therapy or brachytherapy wherein a radioactive source is placed inside the body close to cancer cells or a tumor mass.

Cancer therapies and their dosages, routes of administration and recommended usage are known in the art and have been described in such literature as the Physician's Desk Reference (58^{th} ed., 2004).

### 5.3.6 Antibiotics

Antibiotics well known to one of skill in the art can be used in the compositions and methods of the invention. Non-limiting examples of antibiotics include penicillin, cephalosporin, imipenem, axtreonam, vancomycin, cycloserine, bacitracin, chloramphenicol, erythromycin, clindamycin, tetracycline, streptomycin, tobramycin, gentamicin, amikacin, kanamycin, neomycin, spectinomycin, trimethoprim, norfloxacin, rifampin, polymyxin, amphotericin B, nystatin, ketocanazole, isoniazid, metronidazole, and pentamidine.

Antibiotics and their dosages, routes of administration and recommended usage are known in the art and have been described in such literature as the Physician's Desk Reference (58^{th} ed., 2004).

### 5.3.7 Antiviral Agents

Any anti-viral agent well-known to one of skill in the art can be used in the compositions and the methods of the invention. Non-limiting examples of anti-viral agents include proteins, polypeptides, peptides, fusion protein antibodies, nucleic acid molecules, organic molecules, inorganic molecules, and small molecules that inhibit or reduce the attachment of a virus to its receptor, the internalization of a virus into a cell, the replication of a virus, or release of virus from a cell. In particular, anti-viral agents include, but are not limited to, nucleoside analogs (e.g., zidovudine, acyclovir, gangcyclovir, vidarabine, idoxuridine, trifluridine, and ribavirin), foscamet, amantadine, rimantadine, saquinavir, indinavir, ritonavir, alpha-interferons and other interferons, and AZT.

Antiviral agents and their dosages, routes of administration and recommended usage are known in the art and have been described in such literature as the Physician's Desk Reference (58^{th} ed., 2004).

### 5.3.8 Natural Products and their Derivatives

In the present invention, the diarylheptanoid compounds can be administered either alone or in combination with natural products and their derivatives. Natural products and derivatives that have immunomodulatory activity, anti-angiogenic activity, anti-TNFα activitiy, anti-inflammatory activity, anti-infective activity, antiviral activity and/or anticancer activity can be used in combination with the diarylheptanoid compounds of the invention. Many such natural products are present in mixtures and are not fully characterized by their chemical structures and/or properties, e.g., botantical extracts. Some have been functionally characterized with their active ingredients partially identified. Non-limiting examples of such extracts include but are not limited to rosemary extracts, green tea extracts, black tea extracts, orange peel extracts, licorice root extracts, resveratrol compounds and derivatives, Inula extracts, Mexican bamboo extracts, or Huzhang extracts. Purified compounds and derivatives thereof present in such extracts can also be used.

Non-limiting examples of phytochemicals or plant extracts that can be used in combination with the compounds and compositions of the invention are disclosed in U.S. Pat. Nos. 6,498,195, 6,627,623, 6,790,869, international patent publications nos. WO 01/21137, WO 02/39956, which are incorporated herein by reference in their entirety.

### 5.4 Uses of the Compositions and Compounds of the Invention

Adverse health conditions, diseases and disorders which can be prevented, treated, managed, or ameliorated by administering an effective amount of one or more compounds or compositions of the invention include, but are not limited to, proliferative disorders and inflammatory disorders, and symptoms thereof.

### 5.4.1 Proliferative Disorders

The compounds of the invention and compositions comprising said compounds can be used to prevent, treat, manage, or ameliorate a proliferative disorder or one or more symptoms thereof. The present invention provides methods for preventing, treating, managing, or ameliorating one or more symptoms of a non-cancerous disorder associated with cellular hyperproliferation, particularly of epithelial cells (e.g., as in asthma, COPD, pulmonary fibrosis, bronchial hyperresponsiveness, psoriasis, lymphoproliferative disorder, and seborrheic dermatitis), and endothelial cells (e.g., as in restenosis, hyperproliferative vascular disease, Behcet's Syndrome, atherosclerosis, and macular degeneration), said methods comprising administering to a subject in need thereof one or more compounds of the invention. The present invention also provides methods for preventing, managing, treating, or ameliorating a non-cancerous disorder associated with cellular hyperproliferation, said methods comprising of administering to a subject in need thereof one or more compounds of the invention and one or more other therapies (e.g., one or more other prophylactic or therapeutic agents) useful for the prevention, treatment, management, or amelioration of said disorder. Non-limiting examples of such agents include the agents described in section 5.3, supra, and in particular, the immunomodulatory agents described in section 5.3.1, the anti-angiogenic agents described in section 5.3.2, the TNF-α antagonists described in section 5.3.3, the anti-inflammatory agents described in section 5.3.4, the anti-cancer agents described in section 5.4.5, the antibiotics described in section 5.3.6, the anti-viral agents described in section 5.3.7, and the natural products, phytochemicals or botanical extracts described in section 5.3.8. One or more of the compounds of the invention may also be used in combination with an anti-cancer therapy such as radiation therapy as described in section 5.3.5.

In a specific embodiment, the invention provides methods for preventing, managing, treating, or ameliorating a non-cancerous disorder associated with cellular hyperproliferation (e.g., Behcet's Syndrome, sarcoidosis, keloids, pulmonary fibrosis, and renal fibrosis) or one or more symptoms thereof, said methods comprising of administering to a subject in need thereof a prophylactically or therapeutically effective amount of one or more compounds of the invention. In another embodiment, the invention provides methods for preventing, managing, treating, or ameliorating a non-cancerous disorder associated with cellular hyperproliferation (e.g., Behcet's Syndrome, sarcoidosis, keloids, pulmonary fibrosis, renal and fibrosis) or one or more symptoms thereof, said methods comprising of administering to a subject in need thereof a prophylactically or therapeutically effective amount of one or more compounds of the invention and a prophylactically or therapeutically effective amount of one or more other therapies (e.g., one or more prophylactic or therapeutic agents).

The invention encompasses methods for preventing, treating, managing, or ameliorating one or more symptoms of a disorder associated with cellular hyperproliferation in a subject refractory to conventional therapies for such disorder, said methods comprising contacting with or administering to subject a dose of a prophylactically or therapeutically effective amount of one or more compounds of the invention. The present invention also provides methods for preventing, managing, treating, or ameliorating a non-cancerous disorder associated with cellular hyperproliferation in a subject refractory to conventional therapies for such disorder, said methods comprising of administering to a subject in need thereof one or more compounds of the invention and one or more other therapies (e.g., one or more other prophylactic or therapeutic agents) useful for the prevention, treatment, management, or amelioration of said disorder. Non-limiting examples of such propylactic or therapeutic agents include the agents described in section 5.3, supra, and in particular, the immunomodulatory agents described in section 5.3.1, the anti-angiogenic agents described in section 5.3.2, the TNF-α antagonists described in section 5.3.3, the anti-inflammatory agents described in section 5.3.4, the anti-cancer agents described in section 5.3.5, the antibiotics described in section 5.3.6, and the natural products, phytochemicals or botanical extracts described in section 5.3.8. One or more of the compounds of the invention may also be used in combination with an anti-cancer therapy such as radiation therapy as described in section 5.3.5, or surgery.

The present invention provides methods for preventing, treating, managing, or ameliorating cancer or one or more symptoms thereof, said methods comprising administering to a subject in need thereof. The invention also provides methods for preventing, treating, managing, or ameliorating cancer in which one or more compounds of the invention are administered in combination with one or more other therapies (e.g., prophylactic or therapeutic agents) useful for the prevention, treatment, management, or amelioration of cancer or a secondary condition. Non-limiting examples of such therapies include the agents described in section 5.3, supra, and in particular, the immunomodulatory agents described in section 5.3.1, the anti-angiogenic agents described in section 5.3.2, the TNF-α antagonists described in section 5.3.3, the anti-inflammatory agents described in section 5.3.4, the anti-cancer agents described in section 5.3.5, the antibiotics described in section 5.3.6, the anti-viral agents described in section 5.3.7, and the natural products, phytochemicals or botanical extracts described in section 5.3.8, and surgery. One or more of the compounds of the invention may also be used in combination with an anti-cancer therapy such as radiation therapy as described in section 5.3.5.

In a specific embodiment, the invention provides a method of preventing, treating, managing, or ameliorating cancer or one or more symptoms thereof, said method comprising administering to a subject in need thereof a dose of a prophylactically or therapeutically effective amount of one or more compounds of the invention. In another embodiment, the invention provides a method of preventing, treating, managing, or ameliorating cancer or one or more symptoms thereof, said method comprising administering to a subject in need thereof a dose of a prophylactically or therapeutically effective amount of one or more compounds of the invention and a dose of a prophylactically or therapeutically effective amount of one or more therapies (e.g., one or more prophylactic or therapeutic agents) useful for the prevention, treatment, management, or amelioration of cancer, or a secondary condition (e.g., a viral, bacterial, or fungal infection).

The compounds of the invention can be used in an in vitro or ex vivo fashion for the management, treatment or amelioration of certain cancers, including, but not limited to leukemias and lymphomas, such treatment involving autologous stem cell transplants. This can involve a multi-step process in which the subject's autologous hematopoietic stem cells are harvested and purged of all cancer cells, the patient's remaining bone-marrow cell population is then eradicated via the administration of a high dose of a compound of the invention with or without accompanying high dose radiation therapy, and the stem cell graft is infused back into the subject. Supportive care is then provided while bone marrow function is restored and the subject recovers.

One or more of the compounds of the invention may be used as a first, second, third, fourth, fifth or more line of cancer therapy. The invention provides methods for preventing, treating, managing, or ameliorating cancer or one or more symptoms thereof in a subject refractory to conventional therapies for such a cancer, said methods comprising administering to said subject a dose of a prophylactically or therapeutically effective amount of one or more compounds of the invention. A cancer may be determined to be refractory to a therapy means when at least some significant portion of the cancer cells are not killed or their cell division arrested in response to the therapy. Such a determination can be made either in vivo or in vitro by any method known in the art for assaying the effectiveness of treatment on cancer cells, using the art-accepted meanings of “refractory” in such a context. In a specific embodiment, a cancer is refractory when the number of cancer cells has not been significantly reduced, or has increased.

The invention provides methods for preventing, managing, treating or ameliorating cancer or one or more symptoms thereof in a subject refractory to existing single agent therapies for such a cancer, said methods comprising administering to said subject a dose of a prophylactically or therapeutically effective amount of one or more compounds of the invention and a dose of a prophylactically or therapeutically effective amount of one or more therapies (e.g., one or more prophylactic or therapeutic agents) useful for the prevention, treatment, management, or amelioration of cancer or a secondary condition. The invention also provides methods for preventing, treating, managing, or ameliorating cancer or a secondary condition by administering one or more compounds of the invention in combination with any other therapy(ies) (e.g., radiation therapy, chemotherapy or surgery) to patients who have proven refractory to other treatments but are no longer on this therapy(ies).

The invention provides methods for the prevention, treatment, management, or amelioration of a patient having cancer and immunosuppressed by reason of having previously undergone other cancer therapies. The invention also provides alternative methods for the prevention, treatment, management, or amelioration of cancer where chemotherapy, radiation therapy, hormonal therapy, and/or biological therapy/immunotherapy has proven or may prove too toxic, i.e., results in unacceptable or unbearable side effects, for the subject being treated. Further, the invention provides methods for preventing the recurrence of cancer in patients that have been treated and have no disease activity by administering one or more compounds of the invention.

Cancers that can be prevented, managed, treated or ameliorated in accordance with the methods of the invention include, but are not limited to, neoplasms, tumors (malignant and benign) and metastases, or any disease or disorder characterized by uncontrolled cell growth. The cancer may be a primary or metastatic cancer. Specific examples of cancers that can be prevented, managed, treated or ameliorated in accordance with the methods of the invention include, but are not limited to, cancer of the head, neck, eye, mouth, throat, esophagus, chest, bone, lung, colon, rectum, stomach, prostate, breast, ovaries, kidney, liver, pancreas, and brain. Additional cancers include, but are not limited to, the following: leukemias such as but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma, gallbladder cancers such as adenocarcinoma; cholangiocarcinomas such as but not limited to pappillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell cancer, adenocarcinoma, hypemephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America). It is also contemplated that cancers caused by aberrations in apoptosis can also be treated by the methods and compositions of the invention. Such cancers may include, but not be limited to, follicular lymphomas, carcinomas with p53 mutations, hormone dependent tumors of the breast, prostate and ovary, and precancerous lesions such as familial adenomatous polyposis, and myelodysplastic syndromes.

In a specific embodiment, the cancer that is being prevented, managed, treated or ameliorated in accordance with the method of the invention is skin cancer, prostate cancer, breast cancer, bone cancer, melanoma, lung cancer and ovarian cancer. In another embodiment, the cancer that is being prevented, managed, treated or ameliorated in accordance with the methods of the invention are metastatic tumors including, but not limited to, tumors that have or may metastasize to the bone (non-limiting examples are prostate, breast and lung cancers that have metastasized or have the potential to metastasize to the bone), tumors that have or may metastasize to the lung, tumors that have or may metastasize to the brain, and tumors that have or may metastasize to other organs or tissues of a subject.

### 5.4.2 Inflammatory Disorders

One or more compounds of the invention and compositions comprising of said compounds can be used to prevent, treat, manage, relieve, or ameliorate an inflammatory disorder or one or more symptoms thereof. The compounds of the invention or compositions comprising said compounds may also be administered in combination with one or more other therapies (e.g., one or more other prophylactic or therapeutic agents) useful for the prevention, treatment, management, or amelioration of a condition associated with inflammation (in particular, an inflammatory disorder) or one or more symptoms thereof. Non-limiting examples of such agents include the agents described in section 5.3, supra, and in particular, the immunomodulatory agents described in section 5.3.1, the anti-angiogenic agents described in section 5.3.2, the TNF-α antagonists described in section 5.3.3, the anti-inflammatory agents described in section 5.3.4, the antibiotics described in section 5.3.6, the anti-viral agents described in section 5.3.7, and the natural products, phytochemicals or botanical extracts described in section 5.3.8.

The compounds of the invention or compositions comprising said compounds can be used to prevent, reduce, or eliminate the symptoms and conditions associated with inflammation. The mechanism of inflammation typically involves 4 main symptoms: redness, excess warmth, edema (swelling), and pain. When tissue is damaged by mechanical, chemical, biological, or invading organisms, mast cells release histamine. The histamine stimulates dilation of the blood vessels. The increase in blood volume to the area causes redness and the sensation of warmth. Kinins are released, which potentiate the vasodilation. The vasodilation causes plasma that contains mediators of acute inflammation (complement, C-reactive protein, antibodies, neutrophils, eosinophils, basophils, monocytes, and lymphocytes) to leak into the surrounding tissue. This causes the tissue to look swollen. The loss of plasma from the blood causes blood to become more viscous. The blood platelets and leukocytes start to stick together and clump. Platelet aggregation causes the platelets to release serotonin, which participates in the formation of pain. The damaged tissue and cell membranes cause an influx of calcium into the cells, which activates the enzyme phospholipase A2. Phospholipase A2 acts on the phospholipids to release arachidonic acid and produce the pro-inflammatory agent called platelet-activating factor. Neutrophils containing lipoxygenase create chemotactic compounds from arachidonic acid. This provokes the release of cytokines that potently activate inducible cyclo-oxygenase 2 (COX-2) and inducible nitric oxide synthase (NOS). (Gilman A, Rail T, Nies A, Taylor P eds, Goodman and Gilman's The Pharmacological Basis of Therapeutics, New York, Pergamon Press, 1990. Robak J, Gryglewski R J, Bioactivity of flavonoids, Pol J Pharmacol, 1996, 48:555-564.

In a specific embodiment, the invention provides a method of preventing, treating, managing, or ameliorating a condition associated with inflammation (e.g., an inflammatory disorder) or one or more symptoms thereof, said method comprising contacting with or administering to a subject in need thereof a dose of a prophylactically or therapeutically effective amount one or more compounds of the invention. In another embodiment, the invention provides a method of preventing, treating, managing, or ameliorating a condition associated with inflammation (e.g., an inflammatory disorder) or one or more symptoms thereof, said method comprising administering to a subject in need thereof a dose of a prophylactically or therapeutically effective amount of one or more of compounds of the invention and a dose of a prophylactically or therapeutically effective amount of one or more other therapies (e.g., one or more other prophylactic or therapeutic agents).

The invention provides methods for preventing, managing, treating or ameliorating a condition associated with inflammation (e.g., an inflammatory disorder) or one or more symptoms thereof in a subject refractory to conventional therapies (e.g., methotrexate and a TNF-α antagonist (e.g., REMICADE™ or ENBREL™)) for such condition, said methods comprising administering to said subject a dose of a prophylactically or therapeutically effective amount of one or more compounds of the invention. The invention also provides methods for preventing, treating, managing, or ameliorating a condition associated with inflammation (e.g., an inflammatory disorder) or one or more symptoms thereof in a subject refractory to existing single agent therapies for such a condition, said methods comprising administering to said subject a dose of a prophylactically or therapeutically effective amount of one or more compounds of the invention and a dose of a prophylactically or therapeutically effective amount of one or more other therapies (e.g., one or more other prophylactic or therapeutic agents). The invention also provides methods for preventing, treating, managing, or ameliorating a condition associated with inflammation (e.g., an inflammatory disorder) by administering one or more compounds of the invention in combination with any other therapy(ies) to patients who have proven refractory to other treatments but are no longer on this therapy(ies). The invention also provides alternative methods for the prevention, treatment, management, or amelioration of a condition associated with inflammation (e.g., an inflammatory disorder) where another therapy has proven or may prove too toxic, i.e., results in unacceptable or unbearable side effects, for the subject being treated. Further, the invention provides methods for preventing the recurrence of a condition associated with inflammation (e.g., an inflammatory disorder) in patients that have been treated and have no disease activity by administering one or more compounds of the invention.

Examples of the inflammatory disorders which can be prevented, managed, treated, or ameliorated in accordance with the methods of the invention, include, but are not limited to, asthma, allergic reactions, allergic disorders, inflammatory disorders characterized by type-1 mediated inflammation, inflammatory disorders characterized by type-2 mediated inflammation, fibrotic disease (e.g., pulmonary fibrosis), psoraisis, multiple sclerosis, systemic lupus erythrematosis, chronic obstructive pulmonary disease (COPD), encephilitis, inflammatory bowel disease (e.g., Crohn's disease and ulcerative colitis), ischemic reperfusion injury, Gout, Behcet's disease, septic shock, undifferentiated spondyloarthropathy, undifferentiated arthropathy, arthritis, rheumatoid arthritis juvenile and adult), osteoarthritis, psoriatic arthritis, inflammatory osteolysis, sepsis, meningitis, and chronic inflammation resulting from chronic viral or bacteria infections. In a specific embodiment, the inflammatory disorder which is prevented, treated, managed, or ameliorated in accordance with the methods of the invention is an inflammatory disorder characterized as a type 2-mediated inflammation. Type 2-mediated inflammation is characterized by eosinophilic and basophilic tissue infiltration and/or extensive mast cell degranulation, a process dependent on cross-linking of surface-bound IgE. In another embodiment, the inflammatory disorder which is prevented, treated, managed, or ameliorated in accordance with the methods of the invention is asthma, Behcet's disease, arthritis, chronic obstructive pulmonary disease (COPD), pulmonary fibrosis, renal fibrosis, Gout or allergic disorders.

In a specific embodiment, an effective amount of one or more compounds of the invention is administered to a subject in combination with an effective amount of one or more therapies (e.g., prophylactic or therapeutic agents) useful in preventing, treating, managing, or ameliorating asthma or one or more symptoms thereof. Non-limiting examples of such theapies include, but are not limited to, adrenergic stimulants (e.g., catecholamines (e.g., epinephrine, isoproterenol, and isoetharine), resorcinols (e.g., metaproterenol, terbutaline, and fenoterol), saligenins (e.g. salbutamol)), anticholinergics (e.g.,atropine sulfate, atropine methylnitrate, and ipratropium bromide (ATROVENT™)), beta2-agonists (e.g.abuterol (VENTOLIN™ and PROVENTIL™), bitolterol (TORNALATE™), levalbuterol (XOPONEX™), metaproterenol (ALUPENT™), pirbuterol (MAXAIR™), terbutlaine (BRETHAIRE™ and BRETHINE™), albuterol (PROVENTIL™, REPETABS™, and VOLMAX™), formoterol (FORADIL AEROLIZER™), and salmeterol (SEREVENT™ and SEREVENT DISKUS™ )), corticosteroids (e.g., methlyprednisolone (MEDROL™), prednisone (PREDNISONE™ and DELTASONE™), and prednisolone (PRELONE™, PEDIAPRED™)), glucocorticoids (e.g. oral steroids or other systemic or oral steroids, and inhaled gucocoritcoids), other steroids, immunosuppressant agents (e.g. methotrexate and gold salts), leukotriene modifiers (e.g., montelukast (SINGULAIR™), zafirlukast (ACCOLATE™), and zileuton (ZYFLOυ)), mast cell stabilizers (e.g., cromolyn sodium (INTAL™) and nedocromil sodium (TILADE™)), methylxanthines (e.g., theophylline (UNIPHYL™, THEO-DUR™, SLO-BID™, AND TEHO-42™)), and mucolytic agents (e.g., acetylcysteine)).

In a specific embodiment, an effective amount of one or more compounds of the invention is administered to a subject in combination with an effective amount of one or more therapies (e.g., prophylactic or therapeutic agents) useful in preventing, treating, managing, or ameliorating allergies or one or more symptoms thereof. Non-limiting examples of therapies include antimediator drugs (e.g., antihistamine, see Table 2), corticosteroids, decongestants, sympathomimetic drugs (e.g., α-adrenergic and β-adrenergic drugs), theophylline and its derivatives, glucocorticoids, and immunotherapies (e.g., repeated long-term injection of allergen, short course desensitization, and venom immunotherapy).

TABLE 2
H¹ ANTIHISTAMINES

| Chemical class and | | |
|---|---|---|
| representative drugs | Usual daily dosage | |
| Ethanolamine | 25-50 | mg every 4-6 hours |
| Diphehydramine | 0.34-2.68 | mg every 12 hours |
| Clemastine | | |
| Ethylenediamine | 25-50 | mg every 4-6 hours |
| Tripelennamine | | |
| Alkylamine | 4 mg every 4-6 hours; or 8-12 mg of SR | |
| Brompheniramine | form every 8-12 hour | |
| Chlorpheniramine | 4 mg every 4-6 hours; or 8-12 mg of SR | |
| Triprolidine | form every 8-12 hour | |
| (1.25 mg/5 ml) | 2.5 | mg every 4-6 hours |
| Phenothiazine | 25 | mg at bedtime |
| Promethazine | | |
| Piperazine | 25 | mg every 6-8 hours |
| Hydroxyzine | | |
| Piperidines | 10 | mg/d |
| Astemizole | 1-2 | mg every 12 hours |
| (nonsedating) | | |
| Azatadine | 10 | mg/d |
| Cetirzine | 4 | mg every 6-8 hour |
| Cyproheptadine | 60 | mg every 12 hours |
| Fexofenadine | 10 | mg every 24 hours |
| (nonsedating) | | |
| Loratidine | | |
| (nonsedating) | | |

In a specific embodiment, an effective amount of one or more compounds of the invention is administered to a subject in combination with an effective amount of one or more therapies (e.g., prophylactic or therapeutic agents) useful in preventing, treating, managing, or ameliorating COPD or one or more symptoms thereof. Non-limiting examples of such therapies include, but are not limited to, bronchodilators (e.g. short-acting β₂-adrenergic agonist (e.g., albuterol, pirbuterol, terbutaline, and metaproterenol), long-acting β₂-adrenergic agonists (e.g., oral sustained-release albuterol and inhaled salmeterol), anticholinergics (e.g., ipratropium bromide), and theophylline and its derivatives (therapeutic range for theophylline is preferably 10-20 μg/mL)), glucocorticoids, exogenous α₁AT (e.g., α₁AT derived from pooled human plasma administered intravenously in a weekly dose of 60 mg/kg ), oxygen, lung transplantation, lung volume reduction surgery, endotracheal intubation, ventilation support, yearly influenza vaccine and pneumococcal vaccination with 23-valent polysaccharide, exercise, and smoking cessation.

In a specific embodiment, an effective amount of one or more compounds of the invention is administered to a subject in combination with an effect amount of one or more therapies (e.g., prophylactic or therapeutic agents) useful in preventing, treating, managing, or ameliorating pulmonary fibrosis or one or more symptoms thereof. Non-limiting examples of such theapies include, oxygen, corticosteroids (e.g., daily administration of prednisone beginning at 1-1.5 mg/kg/d (up to 100 mg/d) for six weeks and tapering slowly over 3-6 months to a minimum maintenance dose of 0.25 mg/kg/d), cytotoxic drugs (e.g., cyclophosphamide at 100-120 mg orally once daily and azathioprine at 3 mg/kg up to 200 mg orally once daily), bronchodilators (e.g., short- and long-acting β₂-adrenegic agonists, anticholinergics, and theophylline and its derivatives), and antihistamines (e.g., diphenhydramine and doxylamine).

Anti-inflammatory therapies and their dosages, routes of administration and recommended usage are known in the art and have been described in such literature as the Physician's desk Reference (58^{th} ed., 2004).

### 5.5 Compositions and Methods for Administration

The present invention provides compositions for the treatment, prophylaxis, and amelioration of profilerative disorder and inflammatory disorders. In a specific embodiment, a composition comprises one, two, three, four or more compounds of the invention, or a pharmaceutically acceptable salt, solvate, or hydrate thereof. Depending on the manner of use, the compositions of the invention can be, but not limited to, a dietary supplement, a food additive, a pharmaceutical composition, or a cosmetic composition. In another embodiment, a composition of the invention comprises one or more compounds of the invention, or a pharmaceutically acceptable salt, solvate, or hydrate thereof, and one or more prophylactic or therapeutic agents known to be useful for, or having been or currently being used in the prevention, treatment, management, or amelioration of a disorder (e.g., a proliferative disorder or an inflammatory disorder), in addition to a compound of the invention.

In a specific embodiment, a composition comprises one, two, three, four or more compounds of the invention, or a pharmaceutically acceptable salt, solvate, or hydrate thereof, and one, two, three, four or more immunomodulatory agents. In another embodiment, a composition comprises one, two, three, four or more compounds of the invention, or a pharmaceutically acceptable salt, solvate, or hydrate thereof, and one, two, three, four or more anti-angiogenic agents. In yet another embodiment, a composition comprises one, two, three, four or more compounds of the invention, or a pharmaceutically acceptable salt, solvate, or hydrate thereof, and one, two, three, four or more anti-inflammatory agents. In another embodiment, a composition comprises one, two, three, four or more compounds of the invention, or a pharmaceutically acceptable salt, solvate, or hydrate thereof, and one, two, three, four or more anti-cancer agents. In another embodiment, a composition comprises one, two, three, four or more compounds of the invention, or a pharmaceutically acceptable salt, solvate, or hydrate thereof, and one, two, three, four or more anti-viral agents. In another embodiment, a composition comprises one, two, three, four or more compounds of the invention, or a pharmaceutically acceptable salt, solvate, or hydrate thereof, and one, two, three, four or more one or more antibiotics. In another embodiment, a composition comprising one, two, three, four or more compounds of the invention, or a pharmaceutically acceptable salt, solvate, or hydrate thereof, or one or more natural products, phytochemicals, or botanical extracts. In yet another embodiment, a composition comprises one, two, three, four or more compounds of the invention, or a pharmaceutically acceptable salt, solvate, or hydrate thereof, and any combination of one, two, three, or more of each of the following prophylactic or therapeutic agents: an immunomodulatory agent, an anti-angiogenic agent, a botantical extract, an anti-cancer agent, an immunomodulatory agent, anti-angiogenic agent, an anti-inflammatory agent, an anti-viral agent, or an anti-bacterial agent (e.g., an antibiotic).

In various embodiments, depending on the intended use and without limitation, a composition of the invention can be a dietary supplement or a food additive. Generally, a dietary supplement is consumed by a subject independent of any food composition, unlike a food additive which is incorporated into a food composition during the processing, manufacture, preparation, or delivery of the food composition, or just before its consumption. Accordingly, a food composition of the invention provide, in addition to nutrition, a therapeutic or prophylactic function to the consumer. In a specific embodiment, a composition of the invention is a food composition comprising a prophylactically or therapeutically effective amount of one or more prophylactic or therapeutic agents (e.g., a compound of the invention, and other prophylactic or therapeutic agent). In various embodiments, the composition of the invention typically comprises one or more consumable fillers or carriers. The term “consumable” means the filler or carrier that is generally suitable for, or is approved by a regulatory agency of the Federal or a state government, for comsumption by animals, and more particularly by humans. In certain embodiments, the meaning of the term “dietary supplement” or “food additive” is the meaning of those terms as defined by a regulatory agency of the Federal or a state government, including the United States Food and Drug Administraion.

In a specific embodiment, a composition of the invention is a pharmaceutical composition or a single unit dosage form. Pharmaceutical compositions and single unit dosage forms of the invention comprise a prophylactically or therapeutically effective amount of one or more prophylactic or therapeutic agents (e.g., a compound of the invention, or other prophylactic or therapeutic agent), and a typically one or more pharmaceutically acceptable carriers or excipients. In a specific embodiment and in this context, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Examples of suitable pharmaceutical carriers are described in “Remington's Pharmaceutical Sciences” by E. W. Martin.

Typical pharmaceutical compositions and dosage forms comprise one or more excipients. Suitable excipients are well-known to those skilled in the art of pharmacy, and non-limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a patient and the specific active ingredients in the dosage form. The composition or single unit dosage form, if desired, can also contain minor amounts of wetting or emulsifying agents, or pH buffering agents.

Lactose-free compositions of the invention can comprise excipients that are well known in the art and are listed, for example, in the U.S. Pharmocopia (USP) SP (XXI)/NF (XVI). In general, lactose-free compositions comprise an active ingredient, a binder/filler, and a lubricant in pharmaceutically compatible and pharmaceutically acceptable amounts. Preferred lactose-free dosage forms comprise an active ingredient, microcrystalline cellulose, pre-gelatinized starch, and magnesium stearate.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

The pharmaceutical compositions and single unit dosage forms can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Such compositions and dosage forms will contain a prophylactically or therapeutically effective amount of a prophylactic or therapeutic agent preferably in purified form, together with a suitable amount of carrier so as to provide the form for proper administration to the patient. The formulation should suit the mode of administration. In a preferred embodiment, the pharmaceutical compositions or single unit dosage forms are sterile and in suitable form for administration to a subject, preferably an animal subject, more preferably a mammalian subject, and most preferably a human subject.

A pharmaceutical composition of the invention is formulated to be compatible with its intended route of administration. Examples of routes of administration include, but are not limited to, parenteral, e.g., intravenous, intradermal, subcutaneous, oral (e.g., inhalation), intranasal, transdermal (topical), transmucosal, intra-tumoral, intra- synovial and rectal administration. In a specific embodiment, the composition is formulated in accordance with routine procedures as a pharmaceutical composition adapted for intravenous, subcutaneous, intramuscular, oral, intranasal or topical administration to human beings. In a preferred embodiment, a pharmaceutical composition is formulated in accordance with routine procedures for subcutaneous administration to human beings. Typically, compositions for intravenous administration are solutions in sterile isotonic aqueous buffer. Where necessary, the composition may also include a solubilizing agent and a local anesthetic such as lignocamne to ease pain at the site of the injection. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the invention will typically vary depending on their use. For example, a dosage form used in the acute treatment of inflammation or a related disorder may contain larger amounts of one or more of the active ingredients it comprises than a dosage form used in the chronic treatment of the same disease. Also, the prophylactically and therapeutically effective dosage form may vary among different types of cancer. Similarly, a parenteral dosage form may contain smaller amounts of one or more of the active ingredients it comprises than an oral dosage form used to treat the same disease or disorder. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990).

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration. Typical dosage forms of the invention comprise a compound of the invention, or a pharmaceutically acceptable salt, solvate or hydrate thereof lie within the range of from about 1 mg to about 1000 mg per day, given as a single once-a-day dose in the morning but preferably as divided doses throughout the day taken with food.

5.5.1 Oral Dosage Forms

Pharmaceutical compositions that are suitable for oral administration, and orally comsumable compositions including but not limited to dietary supplements of the invention, can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Typical oral dosage forms of the invention are prepared by combining the active ingredient(s) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents. Other ingredients that can be incorporated into the dietary supplement or pharmaceutical compositions of the present invention, may include, but are not limited to, vitamins, amino acids, an antioxidant, a botanical extract, metal salts, and minerals.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical/nutraceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Examples of fillers suitable for use in the pharmaceutical compositions, dietary supplements, and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition, dietary supplement, or dosage form.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. An specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant.

Disintegrants that can be used in pharmaceutical compositions, dietary supplmenents and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and mixtures thereof.

Lubricants that can be used in pharmaceutical compositions, dietary supplmenents, and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions, dietary supplmenents, or dosage forms into which they are incorporated.

### 5.5.2 Delayed Release Dosage Forms

Active ingredients of the invention can be administered by controlled release means or by delivery devices that are well known to those of ordinary skill in the art. Examples include, but are not limited to, those described in U.S. Pat. Nos.: 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled-release.

All controlled-release pharmaceutical products and dietary supplements have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

### 5.5.3 Parenteral Dosage Forms

Parenteral dosage forms can be administered to patients by various routes including, but not limited to, subcutaneous, intravenous (including bolus-injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms of the invention.

### 5.5.4 Transdermal, Topical & Mucosal Dosage Forms

Transdermal, topical, and mucosal dosage forms of the invention include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form lotions, tinctures, creams, emulsions, gels or ointments, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16th and 18th eds., Mack Publishing, Easton Pa. (1980 & 1990).

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

### 5.5.5 Dietary Supplements, Food Additives, Food Compositions

The present invention provides food compositions comprising compositions and compounds of the invention. The term “food compositions of the invention” include any substances—raw, prepared or processed—which are intended for animal or human consumption, in particular by eating or drinking, and which contain nutrients in the form of carbohydrates, proteins and/or fats, and which have been modified by the incorporation of a composition, or at least one, two, three, or four compounds of the invention. A food composition of the invention provides an additional benefit other than its nutritional benefit. The present invention provides food compositions that may be used as an anti-inflammatory agent. As such, it can be used to relieve any adverse health condition that is mediated by NF-κB activation, NF-κB nuclear translocation, and/or binding of NF-κB to DNA, such as but not limited to proliferative disorders and inflammatory disorders. It can also be used to relieve any adverse health condition that is mediated by the action of COX-2 including but not limited to, arthritis, headache, allergic rash, inflammatory bowel syndrome, joint pain, chronic fatigue, fibromyalgia and the like. The present invention also provides food compositions that may be used as to prevent cancer. For example, it can be used as an anti-oxidant in any condition that involves the action of free radicals. The present section discusses the forms and components of food compositions that would be desirable and readily produced given the teachings of the present invention. 200 In one embodiment, a composition of the invention can be a food additive. A food additive can be in solid form or liquid form. For example, a food additive of the invention can be a reconstitutable powder that, when reconstituted with a liquid, such as drinking water, can provide a beverage. In another embodiment, a composition or compound of the invention can be incorporated into other foodstuff, such as but not limited to cooking oil, frying oil, salad oil, margarine, mayonnaise or peanut butter. Oils containing the compounds of the invention can be emulsified and used in a variety of water-based foodstuffs, such as drinks. Accordingly, in one embodiment, a food composition comprising compositions and compounds of the invetion can be a beverage, such as but not limited to fortified mineral water, fortified distilled water, a fruit juice-based beverage, a shake, a milk-based beverage, a dairy product-based beverage, a yoghurt-based beverage, a carbonated water-based beverage, an alcoholic drink, a coffee-based beverage, a green tea-based beverage, a black tea-based beverage, a grain-based beverage, a soybean-based beverage, or a beverage based on plant extracts.

In addition to beverages, the compositions of the present invention may be used as a food additive to be combined with other foodstuff, for example, syrups, starches, grains, or grain flour. Such food composition fortified with the compounds of this invention may be used in the preparation of foodstuffs, such as baked goods, meat products with fillers (e.g., hamburgers, sausages, etc.), cereals, pastas, and soups.

The compositions or compounds of the invention can be included in food compositions which also contain a variety of other beneficial components. The optional components useful herein can be categorized by their healthful benefit or their postulated mode of action. However, it is to be understood that the optional components useful herein can in some instances provide more than one healthful benefit or operate via more than one mode of action. Therefore, classifications herein are made for the sake of convenience and are not intended to limit the component to that particular application or applications listed.

Non-limiting examples of such optional components are essential fatty acids, vitamins and minerals. These components are well known to those of skill in the art. Additional disclosure describing the contents and production of food compositions comrpising such components may be found in e.g., U.S. Pat. No. 5,902,797; U.S. Pat. No. 5,834,048; U.S. Pat. No. 5,817,350; U.S. Pat. No. 5,792,461; U.S. Pat. No. 5,707,657 and U.S. Pat. No. 5,656,312, each of which is incorporated herein by reference in their entirety. Essential fatty acids are involved in cardiovascular health as well as in support of the immune system. An imbalance in these essential fatty acids can lead to poor cholesterol metabolism. Additionally, the immune system function can become impaired, leading to inflammation.

In embodiments where the compositions of the invention are dietary supplements or food additives, vitamins, precursors, and derivatives thereof, minerals, and amino acids can be added to the compositions.

The vitamins may be in either natural or synthetic form. Suitable vitamin compounds include, but are not limited to, Vitamin A (e.g., beta carotene, retinoic acid, retinol, retinoids, retinyl palmitate, retinyl proprionate, etc.), Vitamin B (e.g., niacin, niacinamide, riboflavin, pantothenic acid, etc.), Vitamin C (e.g., ascorbic acid, etc.), Vitamin D (e.g., ergosterol, ergocalciferol, cholecalciferol, etc.), Vitamin E (e.g., tocopherol acetate, etc.), and Vitamin K (e.g., phytonadione, menadione, phthiocol, etc.) compounds. The vitamins may be included as the substantially pure material, or as an extract obtained by suitable physical and/or chemical isolation from natural (e.g., plant) sources.

The role of manganese in driving metalloenzyme manganese-superoxide dismutase (Mn-SOD) has been clearly identified, along with a similar role in other metalloenzyme systems (glutamine synthetase, arginase, and pyruvate carboxylase). Numerous enzyme systems have also been shown to undergo manganese activation, even though they are not manganese metalloenzymes. The manganese-SOD connection may be of special clinical importance, since this form of the metalloenzyme appears to be the sole operative form within the cell's mitochondrial membranes, and thus may play a unique role in protection of the mitochondria and assurance of the body's oxidative energy production system. The inclusion of manganese in a dietary supplement would be desirable. Additional micronutrients that may be used include minerals such as but not limited to manganese, selenium, molybdenum, chromium and potassium.

Stress, exercise, and other conditions create free radicals in the body, which can cause damage to the body's components. To counter the free radicals, the present invention may include the following antioxidants in addition to vitamins C and E discussed above: citrus flavonoids, mixed carotenoids, green tea extract, black tea extract and N-acetylcysteine.

In another embodiment, the compounds and compositions of the invention can be added directly to foods so that an effective amount of the compound is ingested during normal meals. Any methods known to those skilled in the art may be used to add to or incorporate the compositions or compounds into natural or processed foodstuff to make the food composition of the invention. Other optional components in a food additive of the invention include but are not limited to anti-caking agent, dessicant, food preservatives, food coloring, artificial sweetner, etc.

In preferred embodiments, the food additives or food compositions of the present invention comprise from about 0.001% to about 50%, by weight of the compounds of the invention. In fact, even more preferred amounts of the combination in increasing order of preference are from about 0.01% to about 35%, 0.1% to about 20%, 0.1% to about 15%, 1% to about 10%, and 2% to about 7%, by weight of the compounds of the invention.

### 5.5.6 Cosmetic Compositions

In another embodiment, the present invention provides cosmetic compositions comprising compositions and compounds of the invention. Also included is a nonexclusive description of various optional and preferred components useful in embodiments of the present invention. As used herein, “safe and effective amount” means an amount of a compound, component, or composition (as applicable) sufficient to significantly induce a positive effect (e.g., confer a noticeable cosmetic benefit), but low enough to avoid serious side effects, (e.g., undue toxicity or allergic reaction), i.e., to provide a reasonable benefit to risk ratio, within the scope of sound medical judgment.

The cosmetic composition of the present invention is suitable for providing healthful, therapeutic or aesthetic skin benefits by contacting, deposition and/or adhesion to skin and/or hair, or by providing and maintaining body and/or hair hygiene. Suitable cosmetic agents include, but are not limited to those selected from the group consisting of absorbents, anti-acne agents, anti-caking agents, anti-cellulite agents, anti-foaming agents, anti-fungal agents, anti-inflammatory agents, anti-microbial agents, anti-oxidants, antiperspirant/deodorant agents, anti-skin atrophy agents, antiviral agents, anti-wrinkle agents, artificial tanning agents and accelerators, astringents, barrier repair agents, binders, buffering agents, bulking agents, chelating agents, colorants, dyes, enzymes, essential oils, film formers, flavors, fragrances, humectants, hydrocolloids, light diffusers, opacifying agents, optical brighteners, optical modifiers, particulates, perfumes, pH adjusters, sequestering agents, skin conditioners/moisturizers, skin feel modifiers, skin protectants, skin sensates, skin treating agents, skin exfoliating agents, skin lightening agents, skin soothing and/or healing agents, skin detergents, skin thickeners, sunscreen agents, topical anesthetics, vitamins, and combinations thereof.

The cosmetic compositions of the present invention may also comprise a cosmetically-acceptable carrier and any optional components. Suitable carriers are well known in the art and are selected based on the end use application. For example, carriers of the present invention include, but are not limited to, those suitable for application to skin. Preferably, the carriers of the present invention are suitable for application to skin (e.g., sunscreens, creams, milks, lotions, masks, serums, etc.) and nails (e.g., polishes, treatments, etc.). Such carriers are well-known to one of ordinary skill in the art, and can include one or more compatible liquid or solid filler diluents or vehicles which are suitable for application to skin and nails. The exact amount of carrier will depend upon the level of the bonding agent and any other optional ingredients that one of ordinary skill in the art would classify as distinct from the carrier (e.g., other active components). The compositions of the present invention preferably comprise from about 75% to about 99.999%, more preferably from about 85% to about 99.99%, still more preferably from 90% to about 99%, and most preferably, from about 93% to about 98%, by weight of the composition, of a carrier.

The carrier and compositions herein can be formulated in a number of ways, including but not limited to emulsions (in emulsion technology, a composition comprising a “dispersed phase” and a “continuous phase;” the dispersed phase existing as small particles or droplets that are suspended in and surrounded by a continuous phase). For example, suitable emulsions include oil-in-water, water-in-oil, water-in-oil-in-water, oil-in-water-in- oil, and oil-in-water-in-silicone emulsions. Preferred compositions comprise an oil-in-water emulsion.

The cosmetic compositions of the present invention can be formulated into a wide variety of product types, including creams, waxes, pastes, lotions, milks, mousses, gels, oils, tonics, and sprays. Preferred compositions are formulated into lotions, creams, gels, and sprays. These product forms may be used for a number of applications, including, but not limited to, soaps, shampoos, hair, hand and body lotions, cold creams, facial moisturizers, anti-acne preparations, topical analgesics, make-ups/cosmetics including foundations, eyeshadows, lipsticks, and the like. Any additional components required to formulate such products vary with product type and can be routinely chosen by one skilled in the art.

If compositions of the present invention are formulated as an aerosol and applied to the skin as a spray-on product, a propellant is added to the composition. Examples of suitable propellants include chlorofluorinated lower molecular weight hydrocarbons. A more complete disclosure of propellants useful herein can be found in Sagarin, Cosmetics Science and Technology, 2nd Edition, Vol. 2, pp. 443-465 (1972).

The compositions of the present invention may contain a variety of other components such as are conventionally used in a given product type provided that they do not unacceptably alter the benefits of the invention. These optional components should be suitable for application to mammalian skin, that is, when incorporated into the compositions they are suitable for use in contact with human skin without undue toxicity, incompatibility, instability, allergic response, and the like, within the scope of sound medical or formulator's judgment. The CTFA Cosmetic Ingredient Handbook, Second Edition (1992) describes a wide variety of nonlimiting cosmetic and pharmaceutical ingredients commonly used in the skin care industry, which are suitable for use in the compositions of the present invention. Examples of these ingredient classes include: enzymes, surfactants, abrasives, skin exfoliating agents, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents (e.g., resorcinol, sulfur, salicylic acid, erythromycin, zinc, etc.), anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, polymer beads, film formers, fragrances, humectants, opacifying agents, pH adjusters, propellants, reducing agents, sequestrants, skin bleaching agents (or depigmenting, lightening agents) (e.g., hydroquinone, azelaic acid, caffeic acid, kojic acid, ascorbic acid, magnesium ascorbyl phosphate, ascorbyl glucosamine), skin soothing and/or healing agents (e.g., panthenol and derivatives (e.g., ethyl panthenol), aloe vera, pantothenic acid and its derivatives, allantoin, bisabolol, and dipotassium glycyrrhizinate), thickeners, hydrocolloids, particular zeolites, and vitamins and derivatives thereof (e.g. tocopherol, tocopherol acetate, beta carotene, retinoic acid, retinol, retinoids, retinyl palmitate, niacin, niacinamide, and the like).

Further examples of optional components include wetting agents; emollients; moisturizing agents such as glycerol, PEG 400, thiamorpholinone and derivatives thereof, or urea; anti-seborrhoea agents such as S-carboxymethylcysteine, S-benzylcysteamine, the salts and the derivatives thereof; antibiotics such as erythromycin and esters thereof, neomycin, clindamycin and esters thereof, and tetracyclines; antifungal agents such as ketoconazole or 4,5-polymethylene-3-isothiazolidones; agents for promoting the regrowth of the hair, such as minoxidil (2,4-diamino-5-piperidinopyridine 3-oxide) and derivatives thereof, diazoxide (7-chloro-3-methyl-1,2,4-benzothiadiazine 1,1-dioxide) and phenytoin (5,4-diphenylimidazolidine-2,4-dione); non-steroidal anti-inflammatory agents; carotenoids and, in particular, b-carotene; anti-psoriatic agents such as anthraline and derivatives thereof. The cosmetic compositions according to the invention may also contain flavor-enhancing agents, preserving agents such as para-hydroxybenzoic acid esters, stabilizing agents, moisture regulators, pH regulators, osmotic pressure modifiers, emulsifying agents, UV-A and UV-B screening agents, and antioxidants such as butylhydroxyanisole or butylhydroxytoluene.

The compositions of the present invention may include carrier components such as are known in the art. Such carriers can include one or more compatible liquid or solid filler diluents or vehicles that are suitable for application to skin and/or hair.

### 5.5.7 Dosage & Frequency of Administration

The amount of the compound or composition of the invention which will be effective in the prevention, treatment, management, relief, or amelioration of an adverse health condition, a disorder (e.g., a proliferative disorder or an inflammatory disorder), or one or more symptoms thereof will vary with the nature and severity of the disease or condition, and the route by which the active ingredient is administered. The frequency and dosage will also vary according to factors specific for each subject or patient depending on the specific therapy (e.g., therapeutic or prophylactic agents) administered, the severity of the disorder, disease, or condition, the route of administration, as well as age, body, weight, response, and the past medical history of the patient. Effective doses may be extrapolated from dose-response curves derived from in vitro or animal model test systems. Suitable regiments can be selected by one skilled in the art by considering such factors and by following, for example, dosages reported in the literature and recommended in the Physician's Desk Reference (57th ed., 2003).

Exemplary doses of a small molecule include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram).

In general, the recommended daily dose range of a compound of the invention for the conditions described herein lie within the range of from about 0.01 mg to about 1000 mg per day, given as a single once-a-day dose preferably as divided doses throughout a day. In one embodiment, the daily dose is administered twice daily in equally divided doses. Specifically, a daily dose range should be from about 5 mg to about 500 mg per day, more specifically, between about 10 mg and about 200 mg per day. In managing the subject or patient, the therapy should be initiated at a lower dose, perhaps about 1 mg to about 25 mg, and increased if necessary up to about 200 mg to about 1000 mg per day as either a single dose or divided doses, depending on the subject or patient's global response. It may be necessary to use dosages of the active ingredient outside the ranges disclosed herein in some cases, as will be apparent to those of ordinary skill in the art. Furthermore, it is noted that the dietitian, clinician or treating physician will know how and when to interrupt, adjust, or terminate therapy in conjunction with individual patient response.

Different effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the compounds of the invention are also encompassed by the above described dosage amounts and dose frequency schedules. Further, when a subject or patient is administered multiple dosages of a compound of the invention, not all of the dosages need be the same. For example, the dosage administered to the subject or patient may be increased to improve the prophylactic or therapeutic effect of the compound or it may be decreased to reduce one or more side effects that a particular subject or patient is experiencing.

In a specific embodiment, the dosage of the composition of the invention or a compound of the invention administered to prevent, treat, manage, or ameliorate a disorder (e.g., a proliferative disorder or an inflammatory disorder), or one or more symptoms thereof in a patient is about 150 μg/kg, preferably about 250 μg/kg, about 500 μg/kg, about 1 mg/kg, about 5 mg/kg, about 10 mg/kg, about 25 mg/kg, about 50 mg/kg, about 75 mg/kg, about 100 mg/kg, about 125 mg/kg, about 150 mg/kg, or about 200 mg/kg or more of a patient's body weight. In another embodiment, the dosage of the composition of the invention or a compound of the invention administered to prevent, treat, manage, or ameliorate a disorder (e.g., a proliferative disorder or an inflammatory disorder), or one or more symptoms thereof in a patient is a unit dose of 0.1 mg to 20 mg, 0.1 mg to 15 mg, 0.1 mg to 12 mg,0.1 mg to 10 mg, 0.1 mg to 8mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7m g, 0.25 mg to 5 mg, 0.S mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg.

The dosages of prophylactic or therapeutic agents other than compounds of the invention, which have been or are currently being used to prevent, treat, manage, or ameliorate a disorder (e.g., a proliferative disorder or an inflammatory disorder), or one or more symptoms thereof can be used in the combination therapies of the invention. Preferably, dosages lower than those which have been or are currently being used to prevent, treat, manage, or ameliorate a disorder (e.g., a proliferative disorder or an inflammatory disorder), or one or more symptoms thereof are used in the combination therapies of the invention. The recommended dosages of agents currently used for the prevention, treatment, management, or amelioration of a disorder (e.g., a proliferative disorder or an inflammatory disorder), or one or more symptoms thereof can obtained from any reference in the art including, but not limited to, Hardman et al., eds., 1996, Goodman & Gilman's The Pharmacological Basis Of Basis Of Therapeutics 9^{th} Ed, Mc-Graw-Hill, New York; Physician's Desk Reference (PDR) 58^{th} Ed., 2004, Medical Economics Co., Inc., Montvale, N.J., which are incorporated herein by reference in its entirety.

In various embodiments, the therapies (e.g., prophylactic or therapeutic agents) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In preferred embodiments, two or more therapies (e.g., prophylactic or therapeutic agents)are administered within the same patent visit.

In certain embodiments, one or more compounds of the invention and one or more other the therapies (e.g., prophylactic or therapeutic agents)are cyclically administered. Cycling therapy involves the administration of a first therapy (e.g., a first prophylactic or therapeutic agents) for a period of time, followed by the administration of a second therapy (e.g., a second prophylactic or therapeutic agents) for a period of time, followed by the administration of a third therapy (e.g., a third prophylactic or therapeutic agents) for a period of time and so forth, and repeating this sequential administration, i.e., the cycle in order to reduce the development of resistance to one of the agents, to avoid or reduce the side effects of one of the agents, and/or to improve the efficacy of the treatment.

In certain embodiments, administration of the same compound of the invention may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least at least 1 day, 2 days, 3 days, 5 days, 10 days, 15 days, 30 days, 45 days, 2 months, 75 days, 3 months, or 6 months.

In a specific embodiment, the invention provides a method of preventing, treating, managing, or ameliorating a disorder (e.g., a proliferative disorder or an inflammatory disorder), or one or more symptoms thereof, said methods comprising administering to a subject in need thereof a dose of at least 150 μg/kg, preferably at least 250 μg/kg, at least 500 μg/kg, at least 1 mg/kg, at least 5 mg/kg, at least 10 mg/kg, at least 25 mg/kg, at least 50 mg/kg, at least 75 mg/kg, at least 100 mg/kg, at least 125 mg/kg, at least 150 mg/kg, or at least 200 mg/kg or more of one or more compounds of the invention once every 3 days, preferably, once every 4 days, once every 5 days, once every 6 days, once every 7 days, once every 8 days, once every 10 days, once every two weeks, once every three weeks, or once a month.

### 5.6 Biological Asssays

Several aspects of the compositions or compounds of the invention are preferably tested in vitro, in a cell culture system, and in an animal model organism, such as a rodent animal model system, for the desired therapeutic activity prior to use in humans. For example, assays which can be used to determine whether administration of a specific composition or a specific combination of therapies (e.g., a compound of the invention and an immunomodulatory agent) is indicated, include cell culture assays in which a patient tissue sample is grown in culture, and exposed to or otherwise contacted with a composition, and the effect of such composition upon the tissue sample is observed. The tissue sample can be obtained by biopsy from the patient. This test allows the identification of the therapeutically most effective therapy (e.g., prophylactic or therapeutic agent(s)) for each individual patient. In various specific embodiments, in vitro assays can be carried out with representative cells of cell types involved in a disorder (e.g., immune cells or cancer cells), to determine if a composition of the invention has a desired effect upon such cell types. As an alternative to the use of tissue, tissue samples, cancer cell lines cn be used in in vitro assays. Examples of cancer cell lines that can be utilized in in vitro assays include, but are not limited to, the MCF-7 breast cancer cell line, the MCF-7/ADR multi-drug resistant breast cancer cell line, the HT114 human melanoma cell line, the MES/DOX doxorubicenresistant human uterine sarcoma cell line, the HT29 human colorectal cell line, the HCT-1 16 human colorectal cell line, the A549 human lung Carcinoma cell line and the BXPC-3 human pancreas primary adenocarcinoma cell line.

The compositions and compounds of the invention can be assayed for their ability to modulate the activation of various types of immune cells (including T cells, B cells, NK cells, macrophages, and dendritic cells). Activation of immune cells can be determined by measuring, e.g., changes in the level of expression and/or phospharylation of cytokines, and/or cell surface markers. Techniques known to those of skill in the art, including, but not limited to, immunoprecipitation followed by Western blot analysis, ELISAs, flow cytometry, Northern blot analysis, and RT-PCR can be used to measure the expression of cytokines and cell surface markers indicative of activation of the immune cell.

The compositions and compounds of the invention can be assayed for their ability to induce the expression and/or activation of a gene product (e.g., cellular protein or RNA) and/or to induce signal transduction in immune cells, cancer cells, and/or endothelial cells. The induction of the expression or activation of a gene product or the induction of signal transduction pathways in immune cells, cancer cells (in particular tubulin-binding agent resistant cancer cells) and/or endothelial cells can be assayed by techniques known to those of skill in the art including, e.g., ELISAs, flow cytometry, Northern blot analysis, Western blot analysis, RT-PCR kinase assays and electrophoretic mobility shift assays. The compositions and compounds of the invention can also be assayed for their ability to modulate immune cell proliferation, endothelial and cell cancer cell proliferation. Techniques known to those in art, including, but not limited to, ³H-thymidine incorporation, trypan blue cell counts, and fluorescence activated cell sorting (“FACS”) analysis. The compositions and compounds of the invention can also be assayed for their ability to induce cytolysis. Cytolysis can be assessed by techniques known to those in art, including, but not limited to, ⁵¹Cr-release assays. The compositions and compounds of the invention can also be assayed for their ability to inhibit cell migration, cell adhesion angiogenesis or tubulin polymerization using techniques well-known to one of skill in the art or described herein. The compositions and compound can also be assayed for their ability to induce cell cycle arrest or apoptosis.

The compositions and compounds of the invention can be tested in suitable animal model systems prior to use in humans. Such animal model systems include, but are not limited to, rats, mice, chicken, cows, monkeys, pigs, dogs, rabbits, etc. Any animal system well-known in the art may be used. In a specific embodiment of the invention, the compositions and compounds of the invention are tested in a mouse model system. Such model systems are widely used and well-known to the skilled artisan. Pharmaceutical compositions or compounds of the invention can be administered repeatedly. Several aspects of the procedure may vary including, but not limited to, temporal regime for administration of the compositions or compounds.

The anti-cancer activity of the compositions and compounds of the invention can be determined using any suitable animal model, including, but not limited to, SCID mice with a tumor or injected with malignant cells. Examples of animal models for lung cancer include, but are not limited to, lung cancer animal models described by Zhang & Roth (1994, In Vivo 8(5):755-69) and a transgenic mouse model with disrupted p53 function (see, e.g., Morris et al., 1998, J La State Med Soc 150(4):179-85). An example of an animal model for breast cancer includes, but is not limited to, a transgenic mouse that overexpresses cyclin D1 (see, e.g., Hosokawa et al., 2001, Transgenic Res 10(5):471-8). An example of an animal model for colon cancer includes, but is not limited to, a TCR b and p53 double knockout mouse (see, e.g., Kado et al., 2001, Cancer Res 61(6):2395-8). Examples of animal models for pancreatic cancer include, but are not limited to, a metastatic model of Panc02 murine pancreatic adenocarcinoma (see, e.g., Wang et al., 2001, Int J Pancreatol 29(1):37-46) and nu-nu mice generated in subcutaneous pancreatic tumors (see, e.g., Ghaneh et al., 2001, Gene Ther 8(3):199-208). Examples of animal models for non-Hodgkin's lymphoma include, but are not limited to, a severe combined immunodeficiency (“SCID”) mouse (see, e.g., Bryant et al., 2000, Lab Invest 80(4):553-73) and an IgHmu-HOX11 transgenic mouse (see, e.g., Hough et al., 1998, Proc Natl Acad Sci USA 95(23):13853-8). An example of an animal model for esophageal cancer includes, but is not limited to, a mouse transgenic for the human papillomavirus type 16 E7 oncogene (see, e.g., Herber et al., 1996, J Virol 70(3): 1873-81). Examples of animal models for colorectal carcinomas include, but are not limited to, Apc mouse models (see, e.g., Fodde & Smits, 2001, Trends Mol Med 7(8):369-73 and Kuraguchi et al., 2000, Oncogene 19(50):5755-63).

The anti-inflammatory activity of the compositions and compounds of the invention can be determined by using various experimental animal models of inflammatory arthritis known in the art and described in Crofford L. J. and Wilder R. L ., “Arthritis and Autoimmunity in Animals”, in Arthritis and Allied Conditions: A Textbook of Rheumatology, McCarty et al. (eds.), Chapter 30 (Lee and Febiger, 1993). Experimental and spontaneous animal models of inflammatory arthritis and autoimmune rheumatic diseases can also be used to assess the anti-inflammatory activity of the compositions and compounds of the invention. The following are some assays provided as examples and not by limitation.

The principle animal models for arthritis or inflammatory disease known in the art and widely used include: adjuvant-induced arthritis rat models, collagen-induced arthritis rat and mouse models and antigen-induced arthritis rat, rabbit and hamster models, all described in Crofford L. J. and Wilder R. L., “Arthritis and Autoimmunity in Animals”, in Arthritis and Allied Conditions: A Textbook of Rheumatology, McCarty et al. (eds.), Chapter 30 (Lee and Febiger, 1993), incorporated herein by reference in its entirety.

The anti-inflammatory activity of the compositions and compounds of the invention can be assessed using a carrageenan-induced arthritis rat model. Carrageenan-induced arthritis has also been used in rabbit, dog and pig in studies of chronic arthritis or inflammation. Quantitative histomorphometric assessment is used to determine therapeutic efficacy. The methods for using such a carrageenan-induced arthritis model is described in Hansra P. et al., “Carrageenan-Induced Arthritis in the Rat,”Inflammation, 24(2): 141-155, (2000). Also commonly used are zymosan-induced inflammation animal models as known and described in the art.

The anti-inflammatory activity of the compositions and compounds of the invention can also be assessed by measuring the inhibition of carrageenan-induced paw edema in the rat, using a modification of the method described in Winter C. A. et al., “Carrageenan-Induced Edema in Hind Paw of the Rat as an Assay for Anti-inflammatory Drugs” Proc. Soc. Exp. Biol Med. 111, 544-547, (1962). This assay has been used as a primary in vivo screen for the anti-inflammatory activity of most NSAIDs, and is considered predictive of human efficacy. The anti-inflammatory activity of the test composition or compound of the invention is expressed as the percent inhibition of the increase in hind paw weight of the test group relative to the vehicle dosed control group.

In a specific embodiment of the invention where the experimental animal model used is adjuvant-induced arthritis rat model, body weight can be measured relative to a control group to determine the anti-inflammatory activity of the compositions and compounds of the invention. Alternatively, the efficacy of the compositions and compounds of the invention can be assessed using assays that determine bone loss. Animal models such as ovariectomy-induced bone resorption mice, rat and rabbit models are known in the art for obtaining dynamic parameters for bone formation. Using methods such as those described by Yositake et al. or Yamamoto et al., bone volume is measured in vivo by microcomputed tomography analysis and bone histomorphometry analysis. Yoshitake et al., “Osteopontin-Deficient Mice Are Resistant to Ovariectomy-Induced Bone Resorption,” Proc. Natl. Acad. Sci. 96:8156-8160, (1999); Yamamoto et al., “The Integrin Ligand Echistatin Prevents Bone Loss in Ovariectomized Mice and Rats,” Endocrinology 139(3): 1411-1419, (1998), both incorporated herein by reference in their entirety.

Additionally, animal models for inflammatory bowel disease can also be used to assess the efficacy of the pharmaceutical compositions and compounds of the invention (Kim et al., 1992, Scand. J. Gastroentrol. 27:529-537; Strober, 1985, Dig. Dis. Sci. 30(12 Suppl):3S-10S). Ulcerative cholitis and Crohn's disease are human inflammatory bowel diseases that can be induced in animals. Sulfated polysaccharides including, but not limited to amylopectin, carrageen, amylopectin sulfate, and dextran sulfate or chemical irritants including but not limited to trinitrobenzenesulphonic acid (TNBS) and acetic acid can be administered to animals orally to induce inflammatory bowel diseases.

Animal models for asthma can also be used to assess the efficacy of the compositions and compounds of the invention. An example of one such model is the murine adoptive transfer model in which aeroallergen provocation of TH1 or TH2 recipient mice results in TH effector cell migration to the airways and is associated with an intense neutrophilic (TH1) and eosinophilic (TH2) lung mucosal inflammatory response (Cohn et al., 1997, J. Exp. Med. 1861737-1747).

Animal models for psoriasis can also be used to assess the efficacy of the compositions and compounds of the invention. Animal models for psoriasis have been developed (see, e.g., Schon, 1999, J. Invest. Dermatol. 112:405-410).

Further, any assays known to those skilled in the art can be used to evaluate the prophylactic and/or therapeutic utility of the compositions and compounds of the invention for the disorders disclosed herein.

The toxicity and/or efficacy of the compositions and compounds of the invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions and compounds of the invention that exhibit large therapeutic indices are preferred. While compositions and compounds of the invention that exhibit toxic side effects may be used, care should be taken to design a delivery system that targets such compositions and compounds to the site of affected tissue in order to minimize potential damage to uninfected cells and, thereby, reduce side effects.

The data obtained from the cell culture assays and animal studies can be used in formulating a range of dosage of the compositions and compounds of the invention for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography (HPLC) and radioimmunasssay (RIA). The pharmacokinetics of a prophylactic or therapeutic can be determined, e.g., by measuring parameters such as peak plasma level (Cₘₐₓ), area under the curve (AUC, which is measured by plotting plasma concentration of the agent versus time, and reflects bioavailability), half-life of the compound (t_{1/2}), and time at maximum concentration.

Efficacy in preventing or treating a proliferative disorder such as cancer may be demonstrated, e.g., by detecting the ability of the compositions and compounds of the invention to reduce one or more symptoms of the proliferative disorder, to reduce the proliferation of cancerous cells, to reduce the spread of cancerous cells, or to reduce the size of a tumor. Efficacy in preventing or treating an inflammatory disorder may be demonstrated, e.g., by detecting the ability of the compositions and compounds of the invention to reduce one or more symptoms of the inflammatory disorder, to decrease T cell activation, to decrease T cell proliferation, to modulate one or more cytokine profiles, to reduce cytokine production, to reduce inflammation of a joint, organ or tissue or to improve quality of life. Changes in inflammatory disease activity may be assessed through tender and swollen joint counts, patient and physician global scores for pain and disease activity, and the ESR/CRP. Progression of structural joint damage may be assessed by quantitative scoring of X-rays of hands, wrists, and feet (Sharp method). Changes in functional status in humans with inflammatory disorders may be evaluated using the Health Assessment Questionnaire (HAQ), and quality of life changes are assessed with the SF-36.

### 5.7 Article of Manufacture

The invention encompasses an article of manufacture that can simplify the administration of a compound of the invention to a subject. A typical article of manufacture of the invention comprises a unit dosage form of a composition or compound of the invention. In one embodiment, the unit dosage form is a container, preferably a sterile container, containing an effective amount of a composition or compound of the invention and a pharmaceutically acceptable carrier or excipient. The article of manufacture can further comprise a label or printed instructions regarding the use of composition or compound or other informational material that advises the dietitian, physician, technician, consumer, subject, or patient on how to appropriately prevent or treat the disease or disorder in question. In other words, the article of manufacture includes instruction means indicating or suggesting a dosing regimen including, but not limited to, actual doses, monitoring procedures, and other monitoring information. The article of manufacture can also further comprise a unit dosage form of another prophylactic or therapeutic agent, for example, a container containing an effective amount of another prophylactic or therapeutic agent. In a specific embodiment, the article of manufacture comprises a container containing an effective amount of a composition or compound of the invention and a pharmaceutically acceptable carrier or excipient and a container containing an effective amount of another propylactic or therapeutic agent and a pharmaceutically acceptable carrier or excipient. Examples of other prophylactic or therapeutic agents include, but are not limited to, those listed above. As with any pharmaceutical product and dietary supplement, the packaging material and container included in the article of manufacture are designed to protect the stability of the product during storage and shipment.

Article of manufacture of the invention can further comprise devices that are useful for administering the unit dosage forms. Examples of such devices include, but are not limited to, syringes, drip bags, patches, and inhalers.

Articles of manufacture of the invention can further comprise pharmaceutically acceptable vehicles or consumable vehicles that can be used to administer one or more active ingredients (e.g., a compound of the invention). For example, if an active ingredient is provided in a solid form that must be reconstituted for parenteral or oral/enteral administration, the article of manufacture can comprise a sealed container of a suitable vehicle in which the active ingredient can be dissolved. For parenteral administration, a particulate-free sterile solution is preferred. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### 6. EXAMPLES

In this example, the 2,2-diphenyl-1-picrylhydrazyl (DPPH) method was used to explore the capacity of five isolated compounds of the invention to act as free radical scavengers. The DPPH assay has been extensively used to test the free radical scavenging ability of various chemicals (Brand-williams, W. et al., Wiss. Technol. 1995, 28, 25-30; Dinis, T. C. et al., Arch. Biochem. Biophys. 1994, 315, 161-169.)

### 6.1 Materials and Methods

### 6.1.1 General Procedures

¹H NMR and ¹³C NMR spectra were recorded on a VXR-200 instrument. Mass spectra were obtained by using direct probe electron ionization (EI) and by atmospheric pressure chemical ionization (APCI) in the negative-ion mode. Direct probe EI-MS was performed on a Finnigan MAT 8230 high-resolution mass spectrometer (San Jose, Calif.). APCI MS analysis was carried out on a Micromass Platform II system (Micromass Co., MA) equipped with a Digital DECPc XL 560 computer for analysis of data. The ion source temperature was set at 150° C. and the probe temperature was set at 450° C. The sample cone voltage was 10 V and the corona discharge was 3.2 KV. HPLC analysis was performed on a Varian 5500 Liquid Chromatograph pump coupled to a Varian 9065 Polychrom diode array detector (Sugar Land, Tex.). Semi-preparative fractionation of mixture was done on an ABI Spectroflow 400 HPLC pump coupled to an ABI Spectroflow 183 Programmable Absorbance Detector (Ramsey, N.J.). Column chromatography was carried out on Silica gel (32-60 Å, particle size) using a glass chromatography column purchase from Kontes (Vineland, N.J.). Formic acid (FA), methanol, 1-butanol, ethyl acetate, hexanes, water and acetonitrile, were purchased from Fisher Scientific (Springfield, N.J.). All solvents used for extraction and chromatographic analysis were of HPLC grade. Mixture of solvents for HPLC were degassed using a sonic cleaner purchased from Fisher Scientific. The UV absorbance was measured using a spectrophotometer (Milton Roy, Model 301).

### 6.1.2 Plant Material and Cell Line

The rhizomes of Alpinia officinarum were purchased from Nuherbs Co. (Oakland, Calif.). Du-145 prostate tumor cells were obtained from the American Type Culture Collection (ATCC). Cells were maintained at 37° C. in an atmosphere of 5% CO₂ and grown in Roswell Park Memorial Institute (RPMI) media 1640 (GIBCO/BRL) with 10% fetal bovine serum and with 5% penicillin and streptomycin. Cells ere routinely checked and found to be free of contamination by mycoplasma.

### 6.1.3 Extraction and Isolation Procedures

The powdered roots of Alpinia officinarum were extracted with methanol and concentrated under vacuum using rotary evaporation. The dried residue was then partitioned with hexanes, ethyl acetate and 1-butanol successively. The three extracts were subjected to bioassays. It was determined that the ethyl acetate extract was the most potent fraction which induced apoptosis in Du-145 cancer cell lines.

The dried ethyl acetate extract was then chromatographed on a normal phase silica gel column (2.5×30 cm) to perform bioassay-directed fractionation. The extract was first dissolved in methanol and loaded onto the column packing material. The prepared extract was then placed on top of the column and elution was performed using a solvent mixture of chloroform/methanol with an increasing amount of methanol (30:1, 20:1, 10:1, 5:1, 0:100; each 1000 mL). Successive fractions were collected and tested for biological activity. The fraction eluted with 30:1 chloroform/methanol was found to be most active. This fraction was then rechromatographed on a semipreparative Zorbax Rx-C18 reversed phase HPLC column (9.4 mm×240 mm, 5 μm) purchased from MacMod Analytical (Chadds Ford, Pa.). Compounds were eluted by an isocratic solvent system: 55% A, water with 0.05% formic acid (FA); 45% B, ACN (v/v) at a flow rate of 4 mL/min. The wavelength monitored was 205 nm. Successive fractions (1-3) were collected and sent for biological testing again. It was found that all three fractions were able to induce apoptosis.

The 30:1 chloroform/methanol extract was then rechromatographed on a normal phase silica gel column and eluted with hexanes/chloroform 20:80, 10:90, 0:100 successively to get the above three fractions. Fraction 3 was a pure compound. Final separation of pure compounds from fractions 1 and 2 were obtained using semipreparative HPLC on a Zorbax Rx-C18 reversed phase column. Isocratic solvent systems were used for the two fractions: fraction 1, 50:50 (Water with 0.05% FA: ACN), v/v; fraction 2, 55:45, (Water with 0.05% FA: ACN), v/v.

### 6.1.4 Quantification of Diarylheptanoids in Alpinia officinarum

At least five different concentrations were prepared for each purified diarylheptanoid and then each sample was chromatographed using an analytical-reversed phase HPLC column. The calibration curve for each compound was obtained by plotting concentration versus UV absorbance. The ethyl acetate extract of lesser galangal was then subjected to the same HPLC program and the concentration of each diarylheptanoid in the ethyl acetate extract was obtained from the calibration curve. The concentrations of the five diarylheptanoids in the rhizomes of Alpinia officinarum were determined by comparing the amount of each compound to that of plant material (dry basis) used in this study.

### 6.1.5 Cytotoxicity Assessed by Cell Viability Assay

Cellular growth in the presence or absence of experimental agents was determined using the modified method as previously described MTT-microculture tetrazolium assay (World Health Organization (ED.), Medicinal Plants in China, WHO Regional Publications, 1989, 21). Briefly, rapidly growing cells were harvested, counted, and inoculated at the appropriate concentrations (100 μL volume) into 96-well micro-titer plates using a multichannel pipet. After 24 hours, AO-1 to AO-5 were applied in different concentrations to triplicate culture wells, and cultures were incubated for 72 hours at 37° C. MTT ( Sigma, St. Louis, Mo.) was prepared at 2 mg/ml in Phosphate buffered saline (PBS) and 50 μL were added to microculture wells. After 4 hour incubation at 37° C., 250 μL were removed from each well, and 150 μL of 100% DMSO were added to solubilize the MTT-formazan product. After thorough mixing with a mechanical plate mixer, absorbence was measured at 570 nm using a Dynatech microplate reader. Treatment of prostate tumor cell line Du-145 with the various diarylheptanoids resulted in comparable IC₅₀ values (drug concentration in a 50% inhibition of growth). This assay is based on the reduction of MTT tetrazolium salt to a formazan product by the metabolic activity of live cells and which was measured in a multiwell scanning spectrophotometer. Cell line growth and growth inhibition were expressed in terms of mean absorbance units and following the substraction of mean background absorbance.

### 6.1.6 Measurement of Radical-Scavenging Activity by DPPH

This method was adapted from that of Brand-Williams, et al. (Brand-williams, W.; Cuvelier, M. E.; Berset, C. Lebensm. Wiss. Technol. 1995, 28, 25-30.). At least seven different concentrations were prepared for each purified compound. 25 μl pure compound solution and 975 μL DPPH solution (63.4 μM) were mixed together and left in the dark for 30 min. Each sample was triplicated and the values were averaged. The absorbance of the samples was measured at 515 nm against methanol solution without DPPH as the blank reference. The absorbance was measured every 30 min until the reaction reached a plateau. The reaction kinetics were then plotted using the above data and the percentage of DPPH remaining at the steady state was obtained. IC₅₀ values were determined by extrapolation from linear regression analysis.

### 6.2 Results and Discussion

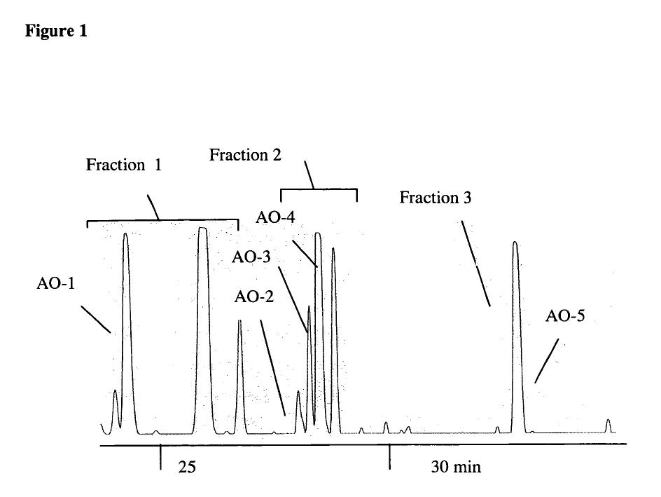

A 30:1 chloroform/methanol fraction of a methanol extract of Alpinia officinarum was analyzed by reverse phase HPLC and its corresponding HPLC chromatogram at 205 nm is presented in FIG. 1. The 30:1 fraction was further subfractionated into three fractions and subjected to the bioassay again. It was shown that all three were active and the second fraction was the most potent.

Fraction 1 obtained from column chromatography was subjected to reverse phase HPLC and three peaks were collected. Peak 2 and 3 were identified to be flavonoids and had no activity at all. Peak 1 showed biological activity to some level and tentatively designated as AO-1. EI mass chromatogram of AO-1 exhibited three major peaks with m/z 310 [M-H₂O⁺., 180 [C₆H₃ (OH)(OMe)CH₂CH₂CHO]⁺., 148 [C₆H₃CH₂CH₂COCH₃]³⁰ ..

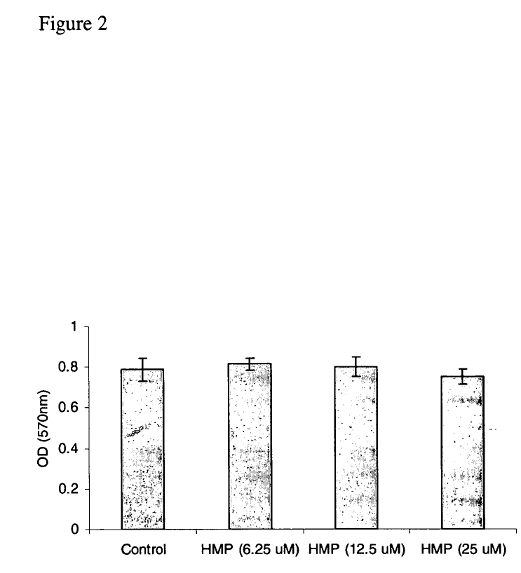

Fraction 2 contained three main peaks and each compound displayed some activity. These were designated as AO-2, AO-3 and AO-4. The EI mass spectrum of AO-2 exhibited peaks at m/z 342 [M]⁺., 310 [M-H₂O]⁺., 205 [C₆H₃(OH)(OMe)CH₂CH₂CHCHCO]⁺., 137 [CH₂C₆H₃ (OH)(OMe)]⁺., 105 [C₈H₉]⁺. and 91 [C₇H₇ ]⁺., which along with the NMR data, indicated the presence of a 4-hydroxy-3 -methoxyphenyl and a phenyl moiety as well as the position of the ketone and aliphatic methoxy group. The EI mass spectrum of AO-3 showed ion at m/z 280 [M]⁺., 133 [C₉H₉O]⁺., 107 [C₇H₇O⁺., 91 [C₇H_{7 ]}⁺, and with the NMR data, indicated the presence of 4-hydroxyphenyl and a phenyl moiety as well as the position of the ketone and α,β-unsaturated ketone grouping. The EI mass spectrum of AO-4 had ion at 310 [M]⁺., 205 [C₆H₃(OH)(OMe)CH₂CH₂CHCHCO]⁺., 137 [CH₂C₆H₃(OH)(OMe)]⁺., 105 [C₈H₉]⁺., 91 [C₇H_{7 ]}⁺., which suggested the same substitution on the benzene ring as sample AO-2. The data also showed the same position of the double bond as well as the ketone group like AO-3. Fraction 3 obtained from column chromatography was a pure compound and exhibited a molecular ion at m/z 264 [M]⁺. as well as a characteristic fragment ion peak at m/z 159 [C₆H₄CH₂CH₂COCHCH_{2 ]}⁺., which indicated the position of the ketone group. On the basis of MS and NMR spectra, AO-1 to AO-5 were identified to be diarylheptanoid compounds and determined to be 5-hydroxy-7-(4″-hydroxy-3″-methoxyphenyl)-1-phenyl-3-heptanone, 5-methoxy-7-(4″-hydroxy-3″-methoxyphenyl)-1-phenyl-3-heptanone, 7-(4″-hydroxyphenyl)-1-phenylhept-4-en-3-one, 7-(4″-hydroxy-3″-methoxyphenyl)-1-penylhept-4-en-3-one and 1,7-diphenylhept-4-en-3-one successively. Structures of these five compounds are shown in FIG. 2. The concentration of AO-1 to AO-5 in the powdered roots of Alpinia officinarum were approximately 134 μg/g, 40 μg/g, 74 μg/g, 168 μg/g, 138 μg/g respectively.

In order to identify the active compounds contributing to the biological activity, the IC₅₀ values for the above five diarylheptanoids were obtained and summarized in Table 1. AO-1 and AO-2 with IC₅₀ 100 and 88 μM were significantly less active than AO-3, AO-4 and AO-5. The IC₅₀ values for AO-3 to AO-5 were 20,μM, 12.5,μM and 32μM respectively. Among the five diarylheptanoids, AO-4 showed the most potent activity followed by AO-3 and AO-5. Therefore the importance of α,β-unsaturated ketone grouping is evident from the fact that substituting the double bond with hydroxy (AO-1) or methoxy (AO-2) groups decreases the biological activity significantly. It also could be seen from the table that substitution at the para position (i.e., 4″) with a hydroxyl group (AO-4) in the phenyl group in position 7 of the carbon backbone enhances the ability to induce apoptosis. Furthermore, substitution at

TABLE 2
Effects of Diarylheptanoids on Inducing
Apoptosis in DU-145 Cancer Cell Line

| Compound | IC₅₀ (μM) |
|---|---|
| AO-1 | 100 |
| AO-2 | 88 |
| AO-3 | 20 |
| AO-4 | 12.5 |
| AO-5 | 32 |

the meta position (i.e., 3″ position) with a methoxy group (AO-4) further enhances the bioactivity. Therefore, substitution on the benzene ring in position 7 of the carbon chain with different functional groups as well as the presence of α,β-unsaturated ketone grouping may significantly affect biological activity.

DPPH, 2,2-Diphenyl-1-picrylhydrazyl (DPPH.), is a stable radical in methanol solution. In its radical form, DPPH. absorbs at 515 nm, but the absorption ceases upon reduction by an antioxidant (AH) or a radical species (R.) (Brand-williams, W.; Cuvelier, M. E.; Berset, C. Lebensm. Wiss. Technol. 1995, 28, 25-30.). The concentration of tested chemicals needed to decrease the initial DPPH. concentration by 50% (IC₅₀) is a parameter widely used to measure the antioxidant potential (Kanner, J.; Frankel, E.; German, B.; Kinsella, J. e. J. Agric. Food Chem. 1994, 42, 64-69, Vinson, J. A.; Dabbagh, Y. A.; Serry, M. M. J. Agric. Food Chem. 1995a, 43,2800-2802.). The lower the IC₅₀, the higher the free radical scavenging power. The IC₅₀ of the above five diarylheptanoids are shown in Table 3.

TABLE 3
IC₅₀ of Diarylheptanoids in Scavenging DPPH Free Radicals

| Compound | IC₅₀ (μM) |
|---|---|
| AO-1 | 11.44 |
| AO-2 | 12.71 |
| AO-3 | More than 1000 |
| AO-4 | 13.7 |
| AO-5 | More than 5000 |

It can be seen from the table that AO-3 and AO-5 have almost no antioxidant power with their IC₅₀ larger than 1000 μm. The introduction of a methoxy group to AO-3's ortho position on the right benzene ring leads to a significant increase of antioxidant activity (AO-4). The substitutions on the right benzene ring of AO-1, AO-2 and AO-4 are the same and their structures' difference comes from the substitution of hydroxy (AO-1) and methoxy (AO-2) to the α,β-unsaturated ketone group (AO-4). Their IC₅₀ concentrations are similar to each other, showing that the presence of the α,β-unsaturated ketone group is not an important factor for the antioxidant activity. However, the introduction of a methoxy group to the ortho position of the phenol ring results in the significant increase of free radical scavenging activity.

### 6.3 Structure Determination of Exemplary Compounds

5-Hydroxy-7-(4″-hydroxy-3″methoxyphenyl)-1-phenyl-3-heptanone referred to herein as AO-1 or compound IIIa-10; MW=328: APCI, m/z 327 [M-H⁻ ion]; EI-MS: 310(12), 205(6), 137(100), 122(10), 105(6), 91(14), 77(7); UV λₘₐₓ nm 279.2; ¹H NMR δ 1.64-1.88 (2H, m, H-6), δ 2.48-2.94 (8H, m, H-1, 2, 4, 7), δ 3.84 (3H, s, 3″-OCH₃) δ 4.22 (1H, m, H-5), δ 6.64 (1H, dd, J=8 Hz, J=2 Hz, H-6″), δ 6.72 (1H, d, J=8 Hz, H-5″), δ 6.78 (1H, d, J=2 Hz, H-2″), δ7.12-7.34 (5H, m, H2′-H6′); ³CNMR δ 28.6 (C-1), δ 30.4 C-7), δ 38.5 (C-6), δ 44.8 (C-2), δ 47.5 (C-4), δ 54.4 (3″-OCH₃), δ 69.7 (C-5), δ 111.2 (C-2″), δ 114.2 (C-5″), δ 119.8 (C-6″), δ 125.6 (C-4′), δ 128.4 (C-2′, 6′), δ128.5 (C-3′, 5′), δ 132.9 (C-1″), δ 140.4 (C-1′), δ 143.8 (C-4″), δ 146.9 (C-3″), δ 209.6 (C-3). (Inoue, Takehisa; Shinbori, T.; Fujioka, M. Yakugaku Zasshi. 1978, 9, 1255-1257, Kikuzaki, H.; Kobayashi, M.; Nakatani, N. Phytochemistry. 1991, 11, 3647-3651)

5-Methoxy-7-(4″-hydroxy-3 ″methoxyphenyl)-1-phenyl-3 -heptanone referred to herein as AO-2 or compound IIIa-122; MW=342: APCI, m/z 341 [M-H⁻ ion]; EI-MS: 342(30), 310(36), 205(8), 177(17), 163(30), 137(100), 105(70), 91(68); UV λₘₐₓ nm: 231, 279.9; ¹H NMR δ 1.6-1.8 (2H, M, H-6), δ 2.4-2.9 (8H, m, H-1, 2, 4, 7), δ 3.28 (3H, s, 5-OCH₃), δ 3.67 (1H, m, H-5), δ 3.84 (3H, s, 3″-OCH₃), δ 6.60 (1H, dd, J=8 Hz, J=2 z, H-6″), δ 6.72 (1H, d, J=8 Hz, H-5″), δ 6.76 (1H, d, J=2 Hz, H-2″), δ 7.1-7.3 (5H, m, H2′-6′); ¹³CNMR δ 29.6 (C-1), δ 30.9 (C-7), δ 36.0 (C-6), 45.0 (C-2), δ 47.5 (C-4), δ 54.4 (3″-OCH₃), δ 56.2 (5-OCH₃), δ 76.0 (C-5), δ 111.2 (C-2″), δ 114.2 (C-5″), 119.8 (C-6″), δ 126.2 (C-4′), δ128.4 (C-2′, 6′), δ 128.5 (C-3′, 5′), δ 132.8 (C-1″), δ 140.6 (C-1′), δ 143.6 (C-4″), δ 146.9 (C-3″), δ 209.1 (C-3). (Kikuzaki, H.; Kobayashi, M.; Nakatani, N. Phytochemistry. 1991, 11, 3647-3651, Kiuchi, F.; Shibuya, M.; Sankawa, U. Chem. Phann. Bull. 1982, 30, 2279-2282.)

7-(4″-hydroxyphenyl)-1-phenyl-hept-4-en-3-one referred to herein as AO-3 or compound IIIb-2; MW=280: APCI, m/z 279; EI-MS: 280(12), 174(5), 159(14), 133(5), 107(100), 105(8), 91(16), 77(10); UV λₘₐₓ nm: 224, 278; ¹H NMR δ 2.48 (2H, 1, J=7.3 Hz, H-6), δ 2.69 (2H, t, J=7.3 Hz, H-7), δ 2.88 (4H, s, H-1, 2), δ 6.10 (1H, d, J=16.2 Hz, H-4), δ 6.71 (2H, d, J=8.4 Hz, H-3″, 5″), δ 6.92 (1H, double t, J=16 Hz, 6 Hz, H-5), δ 7.02 (2H, d, J=8.4 Hz, H-2″, 6″), δ 7.12-7.33 (5H, H2′-6′); ¹³C NMR δ 30.4 (C-1), δ33.6 (C-7), δ 34.7 (C-6), δ 41.4 (C-2), δ 115.2 (C-3″, 5″), δ 126.1 (C-4′), 128.4 (C-2′, 6′), δ 128.5 (C-3′, 5′), δ 129.4 (C-2″, 6″), δ 130.7 (C-4), δ 132.0 (C-1″), δ 140.5 (C-1′), 6 147.3 (C-5), δ 154.7 (C-4″), δ 200.4 (C-3). (Kikuzaki, H.; Kobayashi, M.; Nakatani, N. Phytochemistry. 1991, 11, 3647-3651, Itokawa, H.; Morita, H.; Midorikawa, I. Chem. Phann. Bull. 1985, 33, 4889-4893, and Endo, K.; Kanno, E.; Oshima, Y. Phytochemistry 1990, 29, 797-799.)

7-(4″-hydroxy-3″-methoxyphenyl)-1-phenyl-hept-4-en-3-one referred to herein as AO-4 or compound IIIb-10; MW=310: APCI, m/z 309 [M-H⁻ ion]; EI-MS: 310(18), 205(16), 162(10), 150(4), 138(14), 137(100), 105(7), 91(11); UV λₘₐₓ nm: 224.9, 279.7; ¹H NMR δ 2.51 (2H, q, J=7.3 Hz, H-6), δ 2.72 (2H, t, J=7.3 Hz, H-7), δ 2.88 (4H, s, H-1, 2), δ 3.82 (3H, s, 3″-OCH₃), δ 6.10 (1H, d, J=16 Hz, H-4), δ 6.64 (1H, dd, J=8, J=2 Hz, H-6″), δ 6.72 (1H, d, J=8 Hz, H-5″), δ 6.92 (1H, double t, J=16,-J=6 Hz, H-5), δ 7.10-7.32 (5H, m, H2′-6′); ¹³C NMR δ 29.9 (C-1), δ 33.5 (C-7), δ 34.2 (C-6), δ 41.4 (C-2), δ 54.4 (3″-OCH₃), δ 111.2 (C-2″), δ 114.4 (C-5″), δ 119.9 (C-6″), δ 126.1 (C-4′), δ 128.4 (C-2′, 6′), δ 128.5 (C-3′, 5′), δ 129.9 (C-4), δ 132.4 (C-1″), δ 141.2 (C-1′), δ 143.9 (C-4″), δ 146.5 (C-3″), δ 146.9 (C-5), δ 200.4 (C-3). (Kikuzaki, H.; Kobayashi, M.; Nakatani, N. Phytochemistry. 1991, 11, 3647-3651, Endo, K.; Kanno, E.; Oshima, Y. Phytochemistry 1990, 29, 797-799, and Itokawa, h.; Morita, M.; Mihashi, S. Chem. Pharm. Bull. 1981, 29, 2383-2385.)

1,7-diphenylhept-4-en-3-one referred to herein as AO-5 or compound IIIb-1; MW=264: APCI, m/z 263 [M-H⁻ ion]; EI-MS: 264(26), 172(8), 159(72), 131(11), 105(11), 91(100), 77(8), 65(14); UV λₘₐₓ nm: 226.6; ¹H NMR δ 2.54 (2H, q, J=7.3 Hz, H-6), δ 2.78 (2H, t, H-7), δ 2.87 (4H, s, H-1, 2), δ 6.10 (1H, d, J=16.0 Hz, H-4), δ 6.80 (1H, double t, J=16 Hz, J=6 Hz, H-5), δ 7.10-7.32 (10H, m, H2′-6′, 2″-6″); ¹³C NMR δ 30.2 (C-1), δ 34.2 (C-7), δ 34.4 (C-6), δ 41.4 (C-2), δ 125.2 (C-4″), 6 125.4 (C-4′), δ 128.4 (C-2′, 2″, 6′, 6″), δ 128.6 (C-3′, 3″, 5′, 5″), δ 130.7 (C-4), δ 140.3 (C-1″), δ 141.2 (C-1′), δ 146.4 (C-5), δ 199.3 (C-3). (Endo, K.; Kanno, E.; Oshima, Y. Phytochemistry 1990, 29, 797-799, Itokawa, h.; Morita, M.; Mihashi, S. Chem. Pharm. Bull. 1981, 29, 2383-2385)

### 6.4 Conclusion

Column chromatography and HPLC has led to the isolation of five diarylheptanoids from the roots of Alpinia officinarum. Bioassays showed that AO-4, or compound IIIb-10 above, was the most potent of these compounds in inducing apoptosis in the Du-145 cancer cell line. The results indicated that the presence of α,β-unsaturated ketone grouping, such as in formula Ib, IIb or IIIb, may significantly affect bioactivity. In addition, DPPH data showed that AO-1 (IIIa-10), AO-2 (IIIa-122), and AO-4 (IIIb-10) could act as very good free radical scavengers.

### 7. EXAMPLE

In this example, a diarylheptanoid of the invention, 7-(4′-hydroxy-3′-methoxyphenyl)-1-phenylhept-4-en-3-one (HMP or AO-4) was tested for its anti-inflammatory properties, specifically, using in vitro model systems of inflammation. As discussed in detail, below, the compound was shown to suppress LPS induced proinflamatory cytokine (IL-1β and TNF-α) production from human PBMCs, NO production from mouse macrophage cells (RAW 264.7), and expression of iNOS and COX-2 genes for mRNA and protein. Furthermore, involvements of MAPK (p44/42 and p38) and transcription factor NF-κB were also studied.

### 7.1 Material and Methods

### 7.1.1 Reagents and Cells

Mouse macrophage cell line (RAW 264.7) was obtained from American Type Culture Collection (ATCC) and grown in Dulbecco'Modified Eagle's medium (DMEM) supplemented with 15% fetal bovine serum (FBS), penicillin (100 U/ml) and streptomycin (100 μg/ml). DMEM, RPMI-1640 medium, LPS, Tri-Reagent, Ficoll- hypaque, Griess reagent, monoclonal anti β-actin and 3-(4,5-dimethyl-2-thiazolyl)-2,5-diphenyl tetrazolium bromide (MTT) were purchased from Sigma (St. Louis, Mo., USA). IL-1β and TNF-α ELISA kits were purchased from R & D systems (Minneapolis, Minn., USA). The relative RT-PCR kit for mouse iNOS and COX-2 were obtained Ambion Inc (TX, USA) and mouse monoclonal anti iNOS and COX-2 were purchased from BD-Pharmingen, anti-mouse and anti-rabbit IgG conjugated with horseradish peroxidase were purchased from DAKO Corporation (CA, USA). Mouse monoclonal against phosphorylated p44/42 and p38 as well as against total p44/42 and p38 were obtained from Cell Signaling Tech (MA, USA).

### 7.1.2 Isolation and Identification of HMP

A diarylheptanoid from the rhizomes of Alpinia officinarum was isolated by column chromatography and HPLC as described in Section 6. Normal phase column chromatography followed by semi-preparative reversed-phase HPLC was used to isolate this diarylheptanoid, which was identified to be: 7-(4′-hydroxy-3′-methoxyphenyl)-1-phenylhept-4-en-3-one (HMP, or AO-4). This compound was confirmed to be >99% pure by HPLC and NMR studies.

### 7.1.3 MTT Assay

MTT is a pale yellow substrate that is reduced by living cells to yield a dark blue formazan product. This process requires active mitochondria, and even freshly dead cells do not reduce significant amounts of MTT. Mouse macrophage cell line RAW 264.7 were cultured in 96 well flat bottom plate at concentration of 0.25 million/ml and after 12 hrs of preconditioning, various concentrations of diarylheptanoid (HMP) was added and further cultured for 48 hrs. Thereafter, culture medium was aspirated and 100 μl of MTT dye (1 mg/ml in PBS) was added and further incubated for 4 hrs at 37° C. The formazan crystals made due to dye reduction by viable cells were dissolved using acidified isopropanol (0.1 N HCl). Index of cell viability was calculated by measuring the optical density of color produced by MTT dye reduction at 570 nm.

### 7.1.4 Nitric Oxide Measurement

The mouse macrophage cells (RAW 264.7) were cultured in DMEM supplemented with 15% fetal bovine serum (FBS) and antibiotic and antimycotic mixture. The cell suspension of 50,000 cells/well was cultured in flat bottom microtitre plate in quadruplicate for 12 hrs. Thereafter, 100 μl of media was replaced with fresh medium containing either LPS (0.5 μg/ml) alone or LPS with various concentrations of HMP and further cultured for 24 hrs. The culture supernatant was collected at the end of culture for nitrite assay, which was used as a measure of NO production. Culture supernatant (100 μl) was mixed with equal volume of Griess reagent (SIGMA, MO, USA) and the absorbance was measured at 570 nm. The concentration of nitrite (μM) was calculated from standard curve drawn with known concentration of sodium nitrite dissolved in DMEM. The results are presented as mean ±SD of 4 replicates of one representative experiment and this experiment was repeated 5 times.

### 7.1.5 TNF-α and IL-1β ELISA

The PBMCs were separated from peripheral blood of normal healthy human volunteers. Cells suspension of 0.5×10⁶ cells/ml in complete RPMI-1640 was prepared and five well cultures were set up with 10⁵ cells/well. In brief, PBMCs suspension was treated with LPS (10 ng/ml) either alone or in combination of different concentration of HMP (6.25 μM-25 μM). Supernatant was harvested after 18 hrs and stored at −70° C. until tested. The quantity of IL-1β and TNF-α present in supernatants was estimated by ELISA (R&D systems, MN, USA) following manufacturer's instruction. A standard curve was obtained by plotting known concentrations of recombinant human IL-1β and TNF-α vs absorbance at 450 nm. Cytokine concentrations in experimental samples were determined with standard curve drawn and results are presented as concentrations of cytokine in pg/ml in culture supernatant.

### 7.1.6 Preparation of Total Protein Lysate for Western Blotting

At indicated time points, cells were rapidly washed with ice-cold PBS and solubilized in cold lysis buffer containing 10 mM Tris-base, 5 mM EDTA, 50 mM NaCl, 1% Triton X-100, 5 mM phenylmethylsulfonyl fluoride (PMSF), 2 mM sodium orthovanadate, 10 μg/ml leupeptin, 25 μg/ml aprotinin, 1 mM sodium pyrophosphate, and 20% glycerol. After incubation for 30 min on ice, lysates were centrifuged (12,500 rpm, 15 min.) and supernatants were collected and protein concentration in samples was estimated by Bio-rad protein assay reagent (Bio-rad laboratories, CA, USA) following manufacturer's instruction.

### 7.1.7 Western Blotting

Equal amount of protein (40 μg) from each sample was loaded on SDS-polyacrylamide electrophoresis gel (8 and 10% separating gels for iNOS and p42/44^{MAPK}, respectively) and resolved for 1.5 h at 120 V in buffer containing 95 mM Tris-HCl, 960 mM glycine, and 0.5% SDS. After electrophoresis the proteins were transferred to Hybond enhanced chemiluminescence nitrocellulose membrane (Amersham-Pharmacia biotech, NJ, USA) at 200 mA for 2 hrs for iNOS, and for 1 hrs for COX-2, p42/44 and p38 MAPK in a buffer containing 25 mM Tris-base, 192 mM glycine, and 20% methanol. After transfer, the membrane was blocked in PBST (20 mM Sodium Phosphate buffer, pH 7.6, 150 mM NaCl, 0.1% Tween 20) containing 5% nonfat dry milk for 1 hr at room temperature and incubated with primary antibody in the blocking solution at 4° C. overnight. Thereafter, the membrane was washed four times with PBST, incubated with secondary antibody in the blocking solution for 1 hr at room temperature and washed four times with PBST for 5 minutes every time. Specific bands were detected by enhanced chemiluminescence's detection system (Amersham-Pharmacia biotech, NJ, USA) and the membrane was exposed to X-ray film. Densitometry was performed with SCION software.

### 7.1.8 Determination of Relative Change in iNOS and COX-2 mRNA Expression

The RAW 264.7 cells were cultured (10⁶/well) in 6 well plate for 24 hrs and then medium was replaced by fresh medium and different concentrations of HMP either alone or in combination of LPS was added and further cultured for 12 hrs. Total RNA was isolated using Tri-reagent (SIGMA, MO, USA) and 5 μg of this total RNA was reverse transcribed to make cDNA using random hexamer and superscript reverse transcriptase (Invitrogen, CA, USA), following manufacturer's instruction. Linear range of amplification of iNOS and COX-2 cDNA (2 μl for COX-2 and 1μl for iNOS of RT reaction mixture) was determined using gene specific primers from Ambion Inc (TX, USA), following manufacturer's instruction. The optimum amount of 18S primer and competitor for iNOS and COX-2 gene were also determined as per manufacturer's instruction. The PCR for iNOS (2 μl cDNA, 30 cycles) and COX-2 (1 μl cDNA, 25 cycles) was performed in a final volume of 50 μl containing dNTPs (each at 2.5 mM), 1× PCR buffer, 5 units of Taq DNA polymerase 0.4 μM of gene specific primer, and optimum ratio of 18S primer and competitor (3:7). After an initial denaturation for 30 sec at 94° C., aforementioned cycles of amplification (95° C. for 30 s, 55° C. for 45 s and 72° C. for 45 s) were performed followed a 10 min final extension at72° C. Finally, PCR products from each sample (10 μl) was resolved in 2% agarose gel (Fisher Biotech, NJ, USA), stained with ethidium bromide and image of gels were captured at appropriate exposure time and magnification. Densitometric analysis was performed using image analysis software (Scion, NIH).

### 7.1.9 Electrophoresis Mobility Shift Assay (EMSA)

RAW 264.7 cells were treated with either LPS (0.5 μg/ml) alone or with various concentration of HMP for 2 hrs. Thereafter, nuclear extracts were prepared using a modified method (Lahti et al., 2000 294:1188-1194). Briefly, cells were washed once with PBS (pH 7.2) and were suspended in hypotonic buffer A [10 mM HEPES (pH 7.6), 10 mM KCl, 0.1 mM EDTA, 1 mM DTT, 0.5 mM PMSF] for 10 min on ice, and vortexed for 10 sec. Nuclei were pelleted by centrifugation at 12,000 g for 5 minutes. Then the pellets were suspended in buffer B [20 mM HEPES (pH 7.6), 25% glycerol, 0.4 M NaCl, 1 mM EDTA, 1 mM DTT, 0.5 mM PMSF] for 30 minutes on ice. The supernatants containing nuclear proteins were collected by centrifugation at 12,000 g for 20 minutes and stored at −80° C. For electrophoretic mobility shift assays, 6 μg of each nuclear extract was mixed with the ³²P-labeled double stranded NF-kB binding consensus oligonucleotides (5′-AGTTGAGGGGACTTTCCCAGGC-3′) (Promega, Wis., USA), and incubated at room temperature for 20 minutes. The incubation mixture contains 1 μg of poly (dI-dC) in a binding buffer [25 mM HEPES (pH 7.9), 0.5 mM EDTA, 0.5 mM DTT, 1% Nonidet P-40, 5% glycerol, and 50 mM NaCl]. The DNA/protein complex was electrophoresed on 5% non-denaturing polyacrylamide gels in Tris/Acetate/EDTA buffer (0.04 M Tris, 0.04 M acetate, 0.002 M EDTA). The specificity of binding was also examined by competition with the unlabeled oligonucleotides. Mobility shift of DNA due to binding of NF-κB complex was detected by Phosphor Imager-445 SI (Molecular Dynamics, Amarsham-Pharmacia, NJ, USA).

### 7.1.10 Statistical Analysis

Data are expressed as mean±standard deviation of the mean (SD). Significance differences between means were determined by Student's t-test. The significance level was set at p<0.05.

### 7.2 Results

### 7.2.1 Effect of HMP on RAW 264.7 Cell Viability

In order to find out whether or not HMP is cytotoxic, RAW 264.7 cells were treated with various concentrations (6.25 μM-25 μM) of HMP for 48 hrs and cell viability assay was performed using MTT dye as described in the materials and methods section, above. HMP showed almost no cytotoxic effect on RAW 264.7 cells. No significant difference in viability of cells treated with HMP (6.25 μM to 25 μM) in comparison to control was observed (FIG. 2).

### 7.2.2 Inhibition of LPS Induced Nitric Oxide Production from Mouse Macrophage RAW 264.7 Cells by HMP

The role of NO in pathogenesis of various inflammatory diseases is well known. The endotoxins such as LPS have been shown to stimulate NO release from macrophages, which play an important role in inflammation. Since the half-life of nitric oxide (NO) is very short, nitrite was measured as an indicator of NO released in LPS-activated macrophages to investigate the anti-inflammatory effects of HMP from Alpinia officinarum. The concentration of nitrite (μM) in culture supernatant after 24 hrs of treatment with LPS (0.5 μg/ml) either alone or with various doses of HMP (6.25-25 μM) was determined using Griess reagent. It was observed that LPS induced production of nitric oxide is significantly inhibited by HMP in a dose dependent manner (Table 4). However, HMP alone has no effect on NO production. More importantly, even the lowest dose of HMP was also able to inhibit the nitric oxide production significantly (p<0.05).

TABLE 4
Effect of HMP on LPS induced NO, IL-1β,
and TNF-α production.

| Treatment | Nitrite | IL-1β | TNF-α |
|---|---|---|---|
| group | (μM) | (pg/ml) | (pg/ml) |
| Control | 3.77 ± 0.82 | 21.8 ± 0.395 | <15.62 |
| LPS | 20.04 ± 3.7 | 512.01 ± 20.68 | 290.97 ± 2.41 |
| HMP | 3.66 ± 1.08 | 22.12 ± 3.33 | <15.62 |
| (6.25 μM) | | | |
| HMP | 14.54 ± 2.5* | 339.21 ± 10.53* | 342.24 ± 11.50 |
| (6.25 μM) + | | | |
| LPS | | | |
| HMP | 3.88 ± 1.14 | 24.45 ± 1.03 | <15.62 |
| (12.5 μM) | | | |
| HMP | 13.75 ± 2.65* | 113.43 ± 3.81* | 298.49 ± 5.47 |
| (12.5 μM) + | | | |
| LPS | | | |
| HMP | 3.86 ± 0.97 | 23.28 ± 1.41 | <15.62 |
| (25 μM) | | | |
| HMP | 9.95 ± 1.41** | 77.27 ± 4.6** | 165.38 ± 8.48* |
| (25 μM) + | | | |
| LPS | | | |

Represents the statistical significance of difference between HMP + LPS and LPS alone.
*p < 0.05;
**p < 0.01.

For the NO assay, RAW 264.7 cells were treated with LPS (0.5 μg/ml) and/or various concentrations of HMP for 24 hrs and amount of nitrite in supernatant from each treatment group was measured using Griess reagent. Each data point represents mean+standard deviation (SD) of 5 replicates of one representative experiment of total 6 experiments.

The amounts of the cytokines IL-1β and TNF-α were measured in supernatants of human PBMCs (0.5×10⁶/ml) cultured with LPS (10 ng/ml) and/or various concentrations of HMP (6.25-25 μM) for 18 hrs by ELISA. Each data point represents mean+standard deviation (SD) of 3 replicates. This experiment was repeated 3 times with similar observations.

### 7.2.3 Suppression of LPS Induced Secretion of Proinflammatory Cytokines (IL-1β and TNF-α) by HMP

The production of proinflammatory cytokines from LPS induced human PBMCs in vitro has been shown earlier (Burkart et al., 2002, J Phannacol Exp Ther). In addition to a suppressive effect of HMP on NO release from RAW 264.7 cells, effects of HMP on LPS induced secretion of proinflamatory cytokines IL-1β and TNF-α from human PBMCs were also measured. The amount of IL-1β and TNF-α in culture supernatant of human PBMCs after 18 hrs of treatment with LPS in presence or absence of various doses of HMP (6.25-25 μM) were tested by ELISA. In concordance to NO inhibition, the HMP also inhibited LPS induced secretion of IL-1β significantly in dose dependent fashion (Table 4). However, the inhibition of TNF-α by HMP was only at 25 μM concentration (Table 4). The production of TNF-α from human PBMCs without any treatment (control) or with HMP alone was found to be below than detection limit (15.62 pg/ml).

### 7.2.3 Inhibition of LPS Induced iNOS and COX-2 Protein Expression by HMP

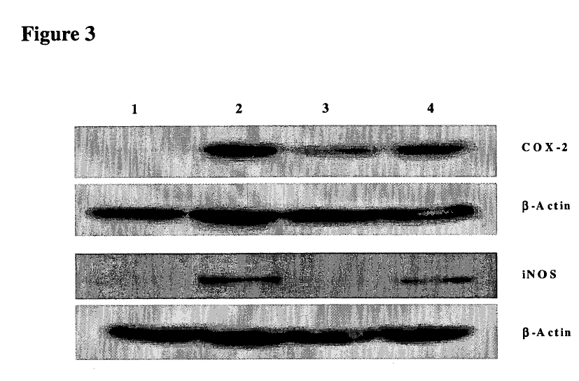

To confirm that the inhibition of NO production is due to less enzymatic activity or decreased protein expression of NOS, the effect of HMP on iNOS protein expression was further studied by Western blotting. In addition to iNOS, the effect of HMP on the expression of COX-2 protein, known to be activated in LPS stimulated macrophages, was also studied. Equal amounts of proteins (40 μg) were resolved to detect the expression of iNOS and COX-2 by Western blot. HMP treatment for 18 hrs markedly inhibited iNOS and COX-2 protein expression in RAW 264.7 cells (FIG. 3). The inhibitory concentration of HMP for iNOS protein expression was similar to that for reduction of NO production. The detection of β-actin was also performed in the same blot as an internal control. This experiment was repeated 4 times with similar observations.

### 7.2.4 Effect of HMP on LPS Induced iNOS and COX-2 mRNA Expression

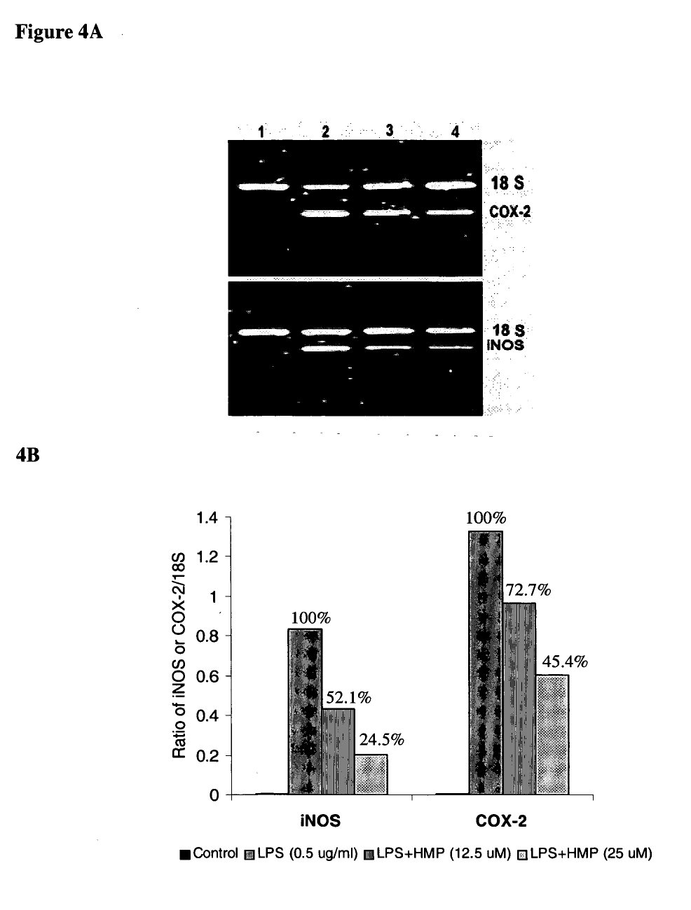

To investigate whether the inhibition of protein expression of iNOS and COX-2 is due to less protein synthesis or due to modulation of post-translational events, RT-PCR analysis for iNOS and COX-2 gene was performed. Using gene specific primers, 2 μl of cDNA was amplified for 349 base pair (bp) of iNOS, 297 bp of COX-2 and 495 bp of 18S ribosomal RNA by PCR as described in the materials and methods section, above. It was observed that various concentrations of HMP (12.5 and 25 μM) inhibited the LPS (0.5 μg/ml) stimulated mRNA expression of iNOS after 12 hrs of treatment with RAW 264.7 cells (FIG. 4A). This inhibition of mRNA correlates to the inhibition of protein expression by HMP. In addition, LPS induced COX-2 mRNA expression is also inhibited by HMP in a dose dependent manner (FIG. 4A). The 18S ribosomal RNA was also amplified in same reaction as an internal control. The densitometric analysis of mRNA expression of iNOS and COX-2 to 18 S RNA was performed by taking the ratios of integrated density of each bands. As shown in FIG. 4B, HMP inhibited LPS induced iNOS expression by 75.5% (at 25 μM) and 47.8% (at 12.5 μM), and COX-2 expression by 54.6% (at 25 μM) and 27.3% (at 12.5 μM).

### 7.2.5 Inhibition of LPS Induced Activation of MAPKs

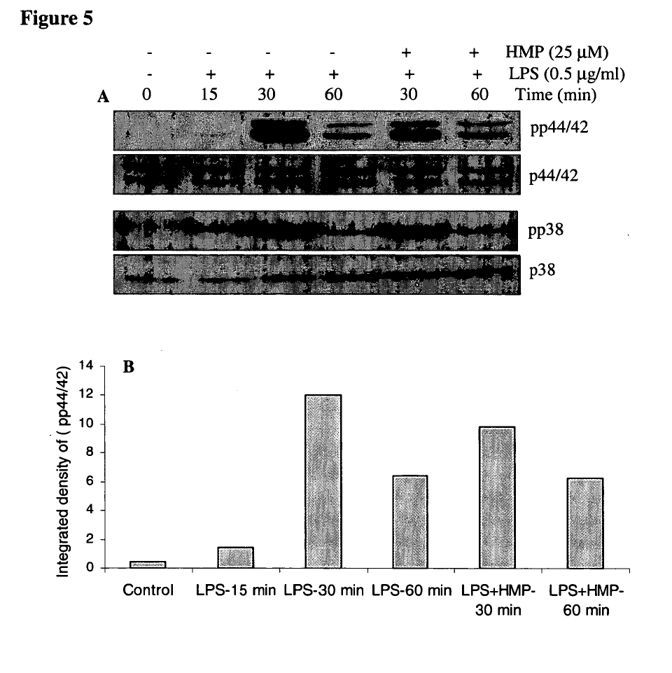

Since p44/42 and p38 MAPKs have been shown to be involved in iNOS induction mediated by LPS in mouse macrophages (Chen et al., 1999, Mol Pharmacol 55:481-488; Lahti et al., 2000, J Phannacol Exp Ther 294:1188-1194), the effects of HMP on the activation of p38 and p44/42 MAPK in LPS-stimulated RAW 264.7 macrophages were investigated. The phosphorylation of threonine and tyrosine residues are required for the activation of MAPK (Raingeaud et al, 1995, J Biol Chem 270:7420-7426). It was demonstrated that activation of p38 and p44/42 by LPS peaked at 30 minutes of LPS treatment and starts declining around 60 min of treatment. When the cells were co-treated with HMP (25 μM) and LPS (0.5 μg/ml) for 30 min and 60 min, the LPS-induced phosphorylation of p44/42 MAPK was markedly inhibited by HMP for 30 min only (FIG. 5). However, no effect of HMP was observed on LPS induced phosphorylation of p38 (FIG. 5).

### 7.2.6 Inhibition of LPS-Induced NF-κB Activation by HMP

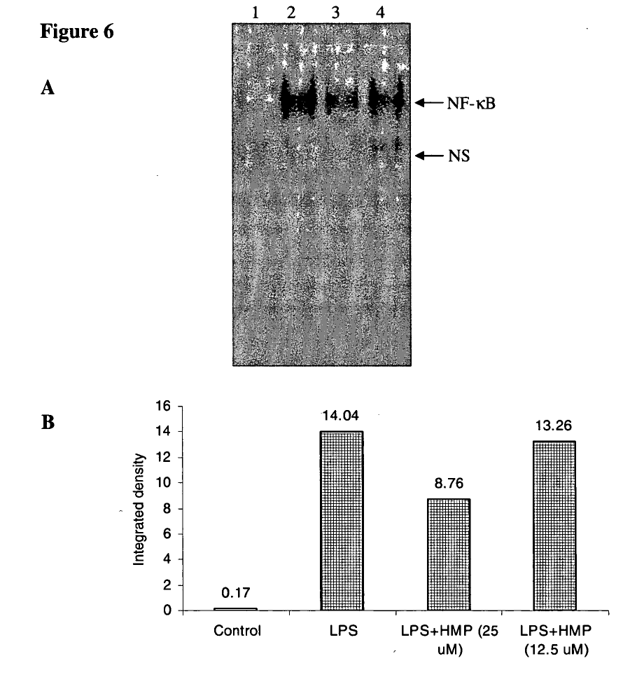

The involvement of transcription factor NF-κB in the expression of iNOS stimulated by proinflamatory cytokines and LPS is well known. Therefore, to investigate the possibility that inhibition of iNOS expression by HMP could be mediated through modulation of NF-κB activation, electrophoretic mobility shift assays (EMSA) were performed. As shown in FIG. 6, the induction of specific NF-κB DNA binding activity by LPS was inhibited by HMP. The relative levels of NF-κB DNA binding activity with the treatment of 12.5 and 25 μM of HMP were less in comparison to LPS alone and marked inhibition was found at 25 μM concentration of HMP. The specificity of binding was examined by competition with the addition of unlabeled/cold oligonucleotides, in excess.

### 7.3 Discussion

The anti-inflammatory properties of a diarylheptanoid (HMP) from Alpinia officinarum was evaluated. The diarylheptanoid compound (HMP) was shown to not be cytotoxic and was shown to inhibit LPS-induced NO production in mouse macrophage RAW 264.7 cells. It was also shown that HMP inhibits proinflamatory cytokine IL-1β production in LPS stimulated human PBMCs, in a dose dependent manner. However, inhibition of LPS stimulated TNF-α was observed only at 25μM concentration of HMP (Table 1). That inhibition of NO production is due to inhibition of iNOS expression at mRNA as well as protein level was shown by RT-PCR and Western blot. In addition, another important mediator of inflammation, COX-2, which acts on arachidonic acid and releases prostaglandins that further orchestrates the process of inflammation (Willoughby et al., 2000, Int J Immunopharmacol) was studied. It has been shown earlier that LPS stimulates COX-2 expression in macrophages (Zhou et al., 2002, J Biol Chem 277:38104-38110). Similar to iNOS inhibition, HMP also inhibits COX-2 protein and mRNA expression in a dose dependent manner as observed by Western and RT-PCR (FIGS. 3 and 4).

The data presented herein indicate that HMP regulates the expression of iNOS by suppressing p44/42 and inhibiting NF-κB. This is an example of the anti-inflammatory properties of a diarylheptanoid, HMP.

Equivalents:

The present invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

Various publications are cited herein, the disclosures of which are incorporated by reference in their entireties.

1. A composition comprising at least one compound having formula Ia or Ib: 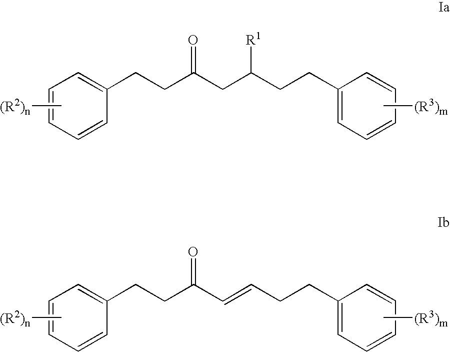
or a pharmaceutically acceptable salt, solvate or hydrate thereof,
wherein
R¹ is hydroxy, alkoxy or acyloxy;
each R²is independently hydroxy, alkoxy or acyloxy;
each R³ is independently hydroxy, alkoxy or acyloxy;
each n is independently an integer from a to 5;
and each m is independently an integer from 0 to 5; and
wherein
the composition is a dietary supplement, food additive, pharmaceutical composition, or a cosmetic composition.

2. The composition of claim 1 wherein the acyloxy is C₂-C₂₂ acyloxy.

3. The composition of claim 1 wherein R¹ is hydroxy, each R² is hydroxy or alkoxy, and each R³ is hydroxy or alkoxy.

4. The composition according to claim 1, wherein the compound having formula Ia or IIb: 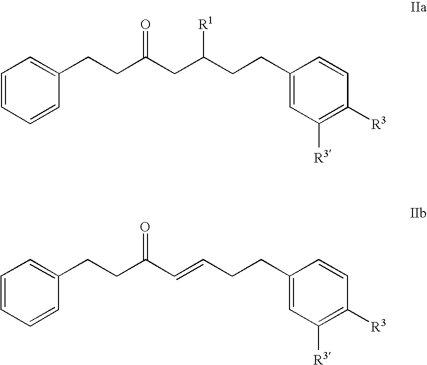
wherein R³′ is is independently hydroxy, alkoxy or acyloxy.

5. The composition of claim 4 wherein the alkoxy is C₁-C₇ alkoxy.

6. The composition of claim 4 wherein the compound is 7-(4″-hydroxy-3″-methoxyphenyl)-1-phenyl-hept-4-en-3-one.

7. The composition of claim 1 comprising more than one compound having the formula Ia or the formula Ib, wherein a first compound having the formula IIa or the formula IIb is present at a concentration greater than about 500 μg/g of the composition.

8. The composition of claim 1 comprising more than one compound having the formula Ia or the formula Ib, wherein a first compound having the formula IIa or the formula IIb constitutes at least 50% of the dry weight of all the compounds having the formula Ia or the formula Ib.

9. The composition of claim 1 comprising more than one compound having the formula Ia or the formula Ib, wherein the ratio of a first compound having the formula IIb relative to a second compound having the formula Ia or the formula Ib but not the formula IIb is greater than 5:1.

10. The composition of claim 1, wherein the composition is a dietary supplement and further comprises at least a consumable carrier, a vitamin, an amino acid, an antioxidant, or a flavoring agent.

11. The composition of claim 1, wherein the composition is a food additive and further comprises at least a consumable carrier, a flavoring agent, a coloring agent, a food preservative, an anti-caking agent, or a dessicant.

12. The composition of claim 1, wherein the composition is a cosmetic composition and further comprises at least a cosmetic carrier, a fragrance, a skin moisturizer, a sunblock agent, or a skin detergent.

13. A method for preventing or treating an adverse health condition in a subject, said adverse health condition being associated with the binding of nuclear factor-kappa B to DNA in cells of said subject, comprising
contacting said subject having said adverse health condition with the composition of claim 1, or administering the composition of claim 1 to said subject having said adverse health condition.

14. The method of claim 13, wherein the composition of claim 1 comprises more than one compound having the formula Ia or the formula Ib, wherein a first compound having the formula IIa or the formula IIb (i) is present at a concentration greater than about 500 μg/g of the composition, or (ii) constitutes at least 50% of the dry weight of all the compounds having the formula Ia or the formula Ib.

15. The method of claim 13, wherein the composition is a pharmaceutical composition and further comprises at least a pharmaceutical carrier, an immunomodulatory agent, an anti-angiogenic agent, a TNF-α antagonist, an anti-inflammatory agent, an anti-cancer agent, an antibiotic, an anti-histamine, or an anti-viral agent.

16. The method of claim 13, wherein said adverse health condition is an inflammatory disorder, a proliferative disorder, or a cancer.

17. A method for relieving a symptom of inflammation in a subject in need thereof, comprising
contacting said subject with the composition of claim 1, or administering the composition of claim 1 to said subject.

18. The method of claim 16, wherein the composition of claim 1 comprises more than one compound having the formula Ia or the formula Ib, wherein a first compound having the formula Ia or the formula IIb (i) is present at a concentration greater than about 500 μg/g of the composition; or (ii) constitutes at least 50% of the dry weight of all the compounds having the formula Ia or the formula Ib.

19. The method of claim 16, wherein the composition is a dietary supplement and further comprises at least a consumable carrier, a vitamin, an amino acid, a metal salt, a mineral, an antioxidant, a botanical extract, or a flavoring agent.

20. The method of claim 16, wherein the inflammation in said subject is associated with asthma, allergic reaction, allergic disorder, fibrotic disease, psoriasis, seborrheic dermatitis, multiple sclerosis, systemic lupus erythrematosis, chronic obstructive pulmonary disease, inflammatory bowel disease, ischemic reperfusion injury, gout, Behcet's disease, septic shock, undifferentiated spondyloarthropathy, undifferentiated arthropathy, arthritis, juvenile rheumatoid arthritis, adult rheumatoid arthritis, osteoarthritis, psoriatic arthritis, inflammatory osteolysis, chronic viral infection or chronic bacterial infection.
